SEQUENCE LISTING (92 sequences)

SEQ ID NO: 1 - protein, 217 aa, Clostridium thermocellum
MIKYKAVFFDFDYTLADSSKAVIECINYALQKMGYPESSPESICRTIGLTLAEAFKILSGDTSDSNADLFRQYFKERADLVMCDRTVMYSTVECVLKKLKKADVKTGIVSTKYRYRIEDILKRDKLLQYFDVIVGGEDVAAHKPDPEGLLKAISMVGCQKEEVLFVGDSTVDARTAKNAGVDFVAVLTGTTGANEFSEYNPGAVIEDLSGLLDMFML

SEQ ID NO: 2 - protein, 217 aa, synthetic construct
MIKYKAVFFDFDYTLADSSKAVIECINYALQKMGYPESSPDSICRTIGLTLAEAFKILTGDTSDSNADLFRQYFKERADLVMCDRTVMFSTVECVLKKLKKADVKTGIVSTKYRYRIEDILKRHKLLQYFDVIVGGEDVTAPKPDPEGLLKAISMVGCQKEEVLFVGDSTVDARTAKNAGVDFVAVLTGTTGANEFSEYNPGAVIEDLSGLLDMFML

SEQ ID NO: 3 - protein, 217 aa, synthetic construct
MIKYKAVFFDFDYTLADSSKAVIECINYALQKMGYPESSPDSICRTIGLTLAEAYKILTGDTSDSNADLFAQYFKERADLVMCDRTVMFSTVECVLKKLKKADVKTGIVSTKYRYRIEDILKRHKLLQYFDVIVGGEDVTAPKPDPEGLLKAISMVGCQKEEVLFVGDSTVDARTAKNAGVDFVAVLTGTTGANEFSEYNPGAVIEDLSGLLDMFML

SEQ ID NO: 4 - protein, 217 aa, synthetic construct
MIKYKAVFFDFDYTLADSSKAVIECINYALQKMGYPESSPDSICRTIGLTLAEAYKILTGDTSDSNADLFAQYFKERADLVMCDRTVMFSTVECVLKKLKKADVKTGIVSTKYRYRIEAILKRHKLLQYFDVIVGGEDVTAPKPDPEGLLKAISMVGCQKEEVLFVGDSTVDARTAKNAGVDFVAVLTGTTGANEFSEYNPGAVIEDLSGLLDMFML

SEQ ID NO: 5 - protein, 217 aa, synthetic construct
MIKYKAVFFDFDYTLADSSKAVIECINYALQKMGYPESSPDSICRTIGLTLAEAYKILTGDTSDSNADLFAQYFKERADLVMCDRTVMFSTVECVLKKLKKADVKTGIVSTKYRYRIEDILKRHKLLQYFDVIIGGEDVTAPKPDPEGLLKAISMVGCQKEEVLFVGDSTVDARTAKNAGVDFVAVLTGTTGANEFSEYNPGAVIEDLSGLLDMFML

SEQ ID NO: 6 - protein, 217 aa, synthetic construct
MIKYKAVFFDFDYTLADSSKAVIECINYALQKMGYPESSPDSICRTIGLTLAEAYKILTGDTSDSNADLFAQYFKERADLVMCDRTVMFSTVECVLKKLKKADVKTGIVSTKYRYRIEDILKRHKLLQYFDVIVGGDDVTAPKPDPEGLLKAISMVGCQKEEVLFVGDSTVDARTAKNAGVDFVAVLTGTTGANEFSEYNPGAVIEDLSGLLDMFML

SEQ ID NO: 7 - protein, 217 aa, synthetic construct
MIKYKAVFFDFDYTLADSSKAVIECINYALQKMGYPESSPDSICRTIGLTLAEAYKILTGDTSDSNADLFAQYFKERADLVMCDRTVMFSTVECVLKKLKQADVKTGIVSTKYRYRIEAILKRHKLLQYFDVIVGGEDVTAPKPDPEGLLKAISMVGCQKEEVLFVGDSTVDARTAKNAGVDFVAVLTGTTGANEFSEYNPGAVIEDLSGLLDMFML

SEQ ID NO: 8 - protein, 217 aa, synthetic construct
MIKYKAVFFDFDYTLADSSKAVIECINYALQKMGYPESSPDSICRTIGLTLAEAYKILTGDTSDSNADLFAQYFKERADLVMCDRTVMFSTVECVLKKLKKADVKTGIVSTKYRYRIEAILKRHKLLQYFDVIIGGEDVTAPKPDPEGLLKAISMVGCQKEEVLFVGDSTVDARTAKNAGVDFVAVLTGTTGANEFSEYNPGAVIEDLSGLLDMFML

SEQ ID NO: 9 - protein, 217 aa, synthetic construct
MIKYKAVFFDFDYTLADSSKAVIECINYALQKMGYPESSPDSICRTIGLTLAEAYKILTGDTSDSNADLFAQYFKERADLVMCDRTVMFSTVECVLKKLKKADVKTGIVSTKYRYRIEAILKRHKLLQYFDVIVGGDDVTAPKPDPEGLLKAISMVGCQKEEVLFVGDSTVDARTAKNAGVDFVAVLTGTTGANEFSEYNPGAVIEDLSGLLDMFML

SEQ ID NO: 10 - protein, 217 aa, synthetic construct
MIKYKAVFFDFDYTLADSSKAVIECINYALQKMGYPESSPDSICRTIGLTLAEAYKILTGDTSDSNADLFAQYFKERADLVMCDRTVMFSTVECVLKKLKQADVKTGIVSTKYRYRIEDILKRHKLLQYFDVIIGGEDVTAPKPDPEGLLKAISMVGCQKEEVLFVGDSTVDARTAKNAGVDFVAVLTGTTGANEFSEYNPGAVIEDLSGLLDMFML

SEQ ID NO: 11 - protein, 217 aa, synthetic construct
MIKYKAVFFDFDYTLADSSKAVIECINYALQKMGYPESSPDSICRTIGLTLAEAYKILTGDTSDSNADLFAQYFKERADLVMCDRTVMFSTVECVLKKLKQADVKTGIVSTKYRYRIEDILKRHKLLQYFDVIVGGDDVTAPKPDPEGLLKAISMVGCQKEEVLFVGDSTVDARTAKNAGVDFVAVLTGTTGANEFSEYNPGAVIEDLSGLLDMFML

SEQ ID NO: 12 - protein, 217 aa, synthetic construct
MIKYKAVFFDFDYTLADSSKAVIECINYALQKMGYPESSPDSICRTIGLTLAEAYKILTGDTSDSNADLFAQYFKERADLVMCDRTVMFSTVECVLKKLKKADVKTGIVSTKYRYRIEDILKRHKLLQYFDVIIGGDDVTAPKPDPEGLLKAISMVGCQKEEVLFVGDSTVDARTAKNAGVDFVAVLTGTTGANEFSEYNPGAVIEDLSGLLDMFML

SEQ ID NO: 13 - protein, 217 aa, synthetic construct
MIKYKAVFFDFDYTLADSSKAVIECINYALQKMGYPESSPDSICRTIGLTLAEAYKILTGDTSDSNADLFAQYFKERADLVMCDRTVMFSTVECVLKKLKQADVKTGIVSTKYRYRIEAILKRHKLLQYFDVIIGGEDVTAPKPDPEGLLKAISMVGCQKEEVLFVGDSTVDARTAKNAGVDFVAVLTGTTGANEFSEYNPGAVIEDLSGLLDMFML

SEQ ID NO: 14 - protein, 217 aa, synthetic construct
MIKYKAVFFDFDYTLADSSKAVIECINYALQKMGYPESSPDSICRTIGLTLAEAYKILTGDTSDSNADLFAQYFKERADLVMCDRTVMFSTVECVLKKLKQADVKTGIVSTKYRYRIEAILKRHKLLQYFDVIVGGDDVTAPKPDPEGLLKAISMVGCQKEEVLFVGDSTVDARTAKNAGVDFVAVLTGTTGANEFSEYNPGAVIEDLSGLLDMFML

SEQ ID NO: 15 - protein, 217 aa, synthetic construct
MIKYKAVFFDFDYTLADSSKAVIECINYALQKMGYPESSPDSICRTIGLTLAEAYKILTGDTSDSNADLFAQYFKERADLVMCDRTVMFSTVECVLKKLKKADVKTGIVSTKYRYRIEAILKRHKLLQYFDVIIGGDDVTAPKPDPEGLLKAISMVGCQKEEVLFVGDSTVDARTAKNAGVDFVAVLTGTTGANEFSEYNPGAVIEDLSGLLDMFML

SEQ ID NO: 16 - protein, 217 aa, synthetic construct
MIKYKAVFFDFDYTLADSSKAVIECINYALQKMGYPESSPDSICRTIGLTLAEAYKILTGDTSDSNADLFAQYFKERADLVMCDRTVMFSTVECVLKKLKQADVKTGIVSTKYRYRIEDILKRHKLLQYFDVIIGGDDVTAPKPDPEGLLKAISMVGCQKEEVLFVGDSTVDARTAKNAGVDFVAVLTGTTGANEFSEYNPGAVIEDLSGLLDMFML

SEQ ID NO: 17 - protein, 217 aa, synthetic construct
MIKYKAVFFDFDYTLADSSKAVIECINYALQKMGYPESSPDSICRTIGLTLAEAYKILTGDTSDSNADLFAQYFKERADLVMCDRTVMFSTVECVLKKLKQADVKTGIVSTKYRYRIEAILKRHKLLQYFDVIIGGDDVTAPKPDPEGLLKAISMVGCQKEEVLFVGDSTVDARTAKNAGVDFVAVLTGTTGANEFSEYNPGAVIEDLSGLLDMFML

SEQ ID NO: 18 - protein, 217 aa, synthetic construct
MIKYKAVFFDFDYTLADSSKAVIECINYALQKMGYPESSPDSICRTIGLTLAEAYKILTGDTSDANADLFAQYFKERADLVMCDRTVMFSTVECVLKKLKQADVKTGIVSTKYRYRIEDILKRHKLLQYFDVIVGGDDVTAPKPDPEGLLKAISMVGCQKEEVLFVGDSTVDARTAKNAGVDFVAVLTGTTGANEFSEYNPGAVIEDLSGLLDMFML

SEQ ID NO: 19 - protein, 217 aa, synthetic construct
MIKYKAVFFDFDYTLADSSKAVIECINYALQKMGYPESSPDSICRTIGLTLAEAYKILTGDTSDANADLFAQYFKERADLVMCDRTVMFSTVECVLKKLKQADVKTGIVSTKYRYRIEAILKRHKLLQYFDVIVGGDDVTAPKPDPEGLLKAISMVGCQKEEVLFVGDSTVDARTAKNAGVDFVAVLTGTTGANEFSEYNPGAVIEDLSGLLDMFML

SEQ ID NO: 20 - protein, 217 aa, synthetic construct
MIKYKAVFFDFDYTLADSSKAVIECINYALQKMGYPESSPDSICRTIGLTLAEAYKILTGDTSDANADLFAQYFKERADLVMCDRTVMFSTVECVLKKLKQADVKTGIVSTKYRYRIEDILKRHKLLQYFDVIIGGDDVTAPKPDPEGLLKAISMVGCQKEEVLFVGDSTVDARTAKNAGVDFVAVLTGTTGANEFSEYNPGAVIEDLSGLLDMFML

SEQ ID NO: 21 - protein, 217 aa, synthetic construct
MIKYKAVFFDFDYTLADSSKAVIECINYALQKMGYPESSPDSICRTIGLTLAEAYKILTGDTSDANADLFAQYFKERADLVMCDRTVMFSTVECVLKKLKQADVKTGIVSTKYRYRIEAILKRHKLLQYFDVIIGGDDVTAPKPDPEGLLKAISMVGCQKEEVLFVGDSTVDARTAKNAGVDFVAVLTGTTGANEFSEYNPGAVIEDLSGLLDMFML

SEQ ID NO: 22 - protein, 217 aa, synthetic construct
MIKYKAVFFDFDYTLADSSKAVIECINYALQKMGYPECSPDSICRTIGLSLAEAFKILTGDTSDSNADLFRQYFKERADLVMCDRTVMYSTVECVLKKLKKADVKTGIVSTKYRYRIEAILKRHKLLQYFDVIVGGEDVTAPKPDPEGLLKAISMVGCQKEEVLFVGDSTVDARTAKNAGVDFVAVLTGTTGANEFSEYNPGAVIPDLSGLLDMFML

SEQ ID NO: 23 - protein, 217 aa, synthetic construct
MIKYKAVFFDFDYTLADSSKAVIECINYALQKMGYPECSPDSICRTIGLTLAEAFKILSGDTSDSNADLFRQYFKERADLVMCDRTVMYSTVECVLKKLKKADVKTGIVSTKYRYRIEAILKRHKLLQYFDVIVGGEDVTAPKPDPEGLNKAISMVGCQKEEVLFVGDSTVDARTAKNAGVDFVAVLTGTTGANEFAEYNPGAVIPDLSGLLDMFML

SEQ ID NO: 24 - protein, 217 aa, synthetic construct
MIKYKAVFFDFDYTLADSSKAVIECINYALQKMGYPECSPDSICRTIGLTLAEAFKILTGDTSDSNADLFAQYFKERADLVMCDRTVMFSTVECVLKKLKKADVKTGIVSTKYRYRIEDILKRHKLLQYFDVIVGGEDVTAPKPDPEGLLKAISMVGCQKEEVLFVGDSTVDARTAKNAGVDFVAVLTGTTGANEFSEYNPGAVIPDLSGLLDMFML

SEQ ID NO: 25 - protein, 223 aa, Clostridium thermocellum
MHHHHHHIKYKAVFFDFDYTLADSSKAVIECINYALQKMGYPESSPESICRTIGLTLAEAFKILSGDTSDSNADLFRQYFKERADLVMCDRTVMYSTVECVLKKLKKADVKTGIVSTKYRYRIEDILKRDKLLQYFDVIVGGEDVAAHKPDPEGLLKAISMVGCQKEEVLFVGDSTVDARTAKNAGVDFVAVLTGTTGANEFSEYNPGAVIEDLSGLLDMFML

SEQ ID NO: 26 - protein, 223 aa, synthetic construct
MHHHHHHIKYKAVFFDFDYTLADSSKAVIECINYALQKMGYPESSPDSICRTIGLTLAEAFKILTGDTSDSNADLFRQYFKERADLVMCDRTVMFSTVECVLKKLKKADVKTGIVSTKYRYRIEDILKRHKLLQYFDVIVGGEDVTAPKPDPEGLLKAISMVGCQKEEVLFVGDSTVDARTAKNAGVDFVAVLTGTTGANEFSEYNPGAVIEDLSGLLDMFML

SEQ ID NO: 27 - protein, 223 aa, synthetic construct
MHHHHHHIKYKAVFFDFDYTLADSSKAVIECINYALQKMGYPESSPDSICRTIGLTLAEAYKILTGDTSDSNADLFAQYFKERADLVMCDRTVMFSTVECVLKKLKKADVKTGIVSTKYRYRIEDILKRHKLLQYFDVIVGGEDVTAPKPDPEGLLKAISMVGCQKEEVLFVGDSTVDARTAKNAGVDFVAVLTGTTGANEFSEYNPGAVIEDLSGLLDMFML

SEQ ID NO: 28 - protein, 223 aa, synthetic construct
MHHHHHHIKYKAVFFDFDYTLADSSKAVIECINYALQKMGYPESSPDSICRTIGLTLAEAYKILTGDTSDSNADLFAQYFKERADLVMCDRTVMFSTVECVLKKLKKADVKTGIVSTKYRYRIEAILKRHKLLQYFDVIVGGEDVTAPKPDPEGLLKAISMVGCQKEEVLFVGDSTVDARTAKNAGVDFVAVLTGTTGANEFSEYNPGAVIEDLSGLLDMFML

SEQ ID NO: 29 - protein, 223 aa, synthetic construct
MHHHHHHIKYKAVFFDFDYTLADSSKAVIECINYALQKMGYPESSPDSICRTIGLTLAEAYKILTGDTSDSNADLFAQYFKERADLVMCDRTVMFSTVECVLKKLKKADVKTGIVSTKYRYRIEDILKRHKLLQYFDVIIGGEDVTAPKPDPEGLLKAISMVGCQKEEVLFVGDSTVDARTAKNAGVDFVAVLTGTTGANEFSEYNPGAVIEDLSGLLDMFML

SEQ ID NO: 30 - protein, 223 aa, synthetic construct
MHHHHHHIKYKAVFFDFDYTLADSSKAVIECINYALQKMGYPESSPDSICRTIGLTLAEAYKILTGDTSDSNADLFAQYFKERADLVMCDRTVMFSTVECVLKKLKKADVKTGIVSTKYRYRIEDILKRHKLLQYFDVIVGGDDVTAPKPDPEGLLKAISMVGCQKEEVLFVGDSTVDARTAKNAGVDFVAVLTGTTGANEFSEYNPGAVIEDLSGLLDMFML

SEQ ID NO: 31 - protein, 223 aa, synthetic construct
MHHHHHHIKYKAVFFDFDYTLADSSKAVIECINYALQKMGYPESSPDSICRTIGLTLAEAYKILTGDTSDSNADLFAQYFKERADLVMCDRTVMFSTVECVLKKLKQADVKTGIVSTKYRYRIEAILKRHKLLQYFDVIVGGEDVTAPKPDPEGLLKAISMVGCQKEEVLFVGDSTVDARTAKNAGVDFVAVLTGTTGANEFSEYNPGAVIEDLSGLLDMFML

SEQ ID NO: 32 - protein, 223 aa, synthetic construct
MHHHHHHIKYKAVFFDFDYTLADSSKAVIECINYALQKMGYPESSPDSICRTIGLTLAEAYKILTGDTSDSNADLFAQYFKERADLVMCDRTVMFSTVECVLKKLKKADVKTGIVSTKYRYRIEAILKRHKLLQYFDVIIGGEDVTAPKPDPEGLLKAISMVGCQKEEVLFVGDSTVDARTAKNAGVDFVAVLTGTTGANEFSEYNPGAVIEDLSGLLDMFML

SEQ ID NO: 33 - protein, 223 aa, synthetic construct
MHHHHHHIKYKAVFFDFDYTLADSSKAVIECINYALQKMGYPESSPDSICRTIGLTLAEAYKILTGDTSDSNADLFAQYFKERADLVMCDRTVMFSTVECVLKKLKKADVKTGIVSTKYRYRIEAILKRHKLLQYFDVIVGGDDVTAPKPDPEGLLKAISMVGCQKEEVLFVGDSTVDARTAKNAGVDFVAVLTGTTGANEFSEYNPGAVIEDLSGLLDMFML

SEQ ID NO: 34 - protein, 223 aa, synthetic construct
MHHHHHHIKYKAVFFDFDYTLADSSKAVIECINYALQKMGYPESSPDSICRTIGLTLAEAYKILTGDTSDSNADLFAQYFKERADLVMCDRTVMFSTVECVLKKLKQADVKTGIVSTKYRYRIEDILKRHKLLQYFDVIIGGEDVTAPKPDPEGLLKAISMVGCQKEEVLFVGDSTVDARTAKNAGVDFVAVLTGTTGANEFSEYNPGAVIEDLSGLLDMFML

SEQ ID NO: 35 - protein, 223 aa, synthetic construct
MHHHHHHIKYKAVFFDFDYTLADSSKAVIECINYALQKMGYPESSPDSICRTIGLTLAEAYKILTGDTSDSNADLFAQYFKERADLVMCDRTVMFSTVECVLKKLKQADVKTGIVSTKYRYRIEDILKRHKLLQYFDVIVGGDDVTAPKPDPEGLLKAISMVGCQKEEVLFVGDSTVDARTAKNAGVDFVAVLTGTTGANEFSEYNPGAVIEDLSGLLDMFML

SEQ ID NO: 36 - protein, 223 aa, synthetic construct
MHHHHHHIKYKAVFFDFDYTLADSSKAVIECINYALQKMGYPESSPDSICRTIGLTLAEAYKILTGDTSDSNADLFAQYFKERADLVMCDRTVMFSTVECVLKKLKKADVKTGIVSTKYRYRIEDILKRHKLLQYFDVIIGGDDVTAPKPDPEGLLKAISMVGCQKEEVLFVGDSTVDARTAKNAGVDFVAVLTGTTGANEFSEYNPGAVIEDLSGLLDMFML

SEQ ID NO: 37 - protein, 223 aa, synthetic construct
MHHHHHHIKYKAVFFDFDYTLADSSKAVIECINYALQKMGYPESSPDSICRTIGLTLAEAYKILTGDTSDSNADLFAQYFKERADLVMCDRTVMFSTVECVLKKLKQADVKTGIVSTKYRYRIEAILKRHKLLQYFDVIIGGEDVTAPKPDPEGLLKAISMVGCQKEEVLFVGDSTVDARTAKNAGVDFVAVLTGTTGANEFSEYNPGAVIEDLSGLLDMFML

SEQ ID NO: 38 - protein, 223 aa, synthetic construct
MHHHHHHIKYKAVFFDFDYTLADSSKAVIECINYALQKMGYPESSPDSICRTIGLTLAEAYKILTGDTSDSNADLFAQYFKERADLVMCDRTVMFSTVECVLKKLKQADVKTGIVSTKYRYRIEAILKRHKLLQYFDVIVGGDDVTAPKPDPEGLLKAISMVGCQKEEVLFVGDSTVDARTAKNAGVDFVAVLTGTTGANEFSEYNPGAVIEDLSGLLDMFML

SEQ ID NO: 39 - protein, 223 aa, synthetic construct
MHHHHHHIKYKAVFFDFDYTLADSSKAVIECINYALQKMGYPESSPDSICRTIGLTLAEAYKILTGDTSDSNADLFAQYFKERADLVMCDRTVMFSTVECVLKKLKKADVKTGIVSTKYRYRIEAILKRHKLLQYFDVIIGGDDVTAPKPDPEGLLKAISMVGCQKEEVLFVGDSTVDARTAKNAGVDFVAVLTGTTGANEFSEYNPGAVIEDLSGLLDMFML

SEQ ID NO: 40 - protein, 223 aa, synthetic construct
MHHHHHHIKYKAVFFDFDYTLADSSKAVIECINYALQKMGYPESSPDSICRTIGLTLAEAYKILTGDTSDSNADLFAQYFKERADLVMCDRTVMFSTVECVLKKLKQADVKTGIVSTKYRYRIEDILKRHKLLQYFDVIIGGDDVTAPKPDPEGLLKAISMVGCQKEEVLFVGDSTVDARTAKNAGVDFVAVLTGTTGANEFSEYNPGAVIEDLSGLLDMFML

SEQ ID NO: 41 - protein, 223 aa, synthetic construct
MHHHHHHIKYKAVFFDFDYTLADSSKAVIECINYALQKMGYPESSPDSICRTIGLTLAEAYKILTGDTSDSNADLFAQYFKERADLVMCDRTVMFSTVECVLKKLKQADVKTGIVSTKYRYRIEAILKRHKLLQYFDVIIGGDDVTAPKPDPEGLLKAISMVGCQKEEVLFVGDSTVDARTAKNAGVDFVAVLTGTTGANEFSEYNPGAVIEDLSGLLDMFML

SEQ ID NO: 42 - protein, 223 aa, synthetic construct
MHHHHHHIKYKAVFFDFDYTLADSSKAVIECINYALQKMGYPESSPDSICRTIGLTLAEAYKILTGDTSDANADLFAQYFKERADLVMCDRTVMFSTVECVLKKLKQADVKTGIVSTKYRYRIEDILKRHKLLQYFDVIVGGDDVTAPKPDPEGLLKAISMVGCQKEEVLFVGDSTVDARTAKNAGVDFVAVLTGTTGANEFSEYNPGAVIEDLSGLLDMFML

SEQ ID NO: 43 - protein, 223 aa, synthetic construct
MHHHHHHIKYKAVFFDFDYTLADSSKAVIECINYALQKMGYPESSPDSICRTIGLTLAEAYKILTGDTSDANADLFAQYFKERADLVMCDRTVMFSTVECVLKKLKQADVKTGIVSTKYRYRIEAILKRHKLLQYFDVIVGGDDVTAPKPDPEGLLKAISMVGCQKEEVLFVGDSTVDARTAKNAGVDFVAVLTGTTGANEFSEYNPGAVIEDLSGLLDMFML

SEQ ID NO: 44 - protein, 223 aa, synthetic construct
MHHHHHHIKYKAVFFDFDYTLADSSKAVIECINYALQKMGYPESSPDSICRTIGLTLAEAYKILTGDTSDANADLFAQYFKERADLVMCDRTVMFSTVECVLKKLKQADVKTGIVSTKYRYRIEDILKRHKLLQYFDVIIGGDDVTAPKPDPEGLLKAISMVGCQKEEVLFVGDSTVDARTAKNAGVDFVAVLTGTTGANEFSEYNPGAVIEDLSGLLDMFML

SEQ ID NO: 45 - protein, 223 aa, synthetic construct
MHHHHHHIKYKAVFFDFDYTLADSSKAVIECINYALQKMGYPESSPDSICRTIGLTLAEAYKILTGDTSDANADLFAQYFKERADLVMCDRTVMFSTVECVLKKLKQADVKTGIVSTKYRYRIEAILKRHKLLQYFDVIIGGDDVTAPKPDPEGLLKAISMVGCQKEEVLFVGDSTVDARTAKNAGVDFVAVLTGTTGANEFSEYNPGAVIEDLSGLLDMFML

SEQ ID NO: 46 - DNA, 672 bp, Clostridium thermocellum
  source 1..672: mol_type = genomic DNA
atgcatcaccatcaccaccacatcaaatacaaagccgtgtttttcgatttcgattataccctggcagatagcagcaaagcagttattgaatgtattaactacgccctgcagaaaatgggttatccggaaagctcaccggaaagcatttgtcgtaccattggtctgaccctggccgaagcattcaaaattctgagcggtgataccagcgatagcaatgcagacctgtttcgtcagtatttcaaagaacgtgcagatctggttatgtgtgatcgtaccgttatgtatagcaccgttgaatgtgttctgaaaaagctgaaaaaagccgatgtgaaaaccggtattgtgagcaccaaatatcgttatcgcattgaggatattctgaaacgcgataaactgctgcagtatttcgatgttattgttggtggtgaagatgtggcagcacataaaccggatcctgaaggtctgctgaaagcaattagcatggttggttgtcagaaagaagaggttctgtttgttggtgattcaaccgttgatgcacgtaccgcaaaaaatgccggtgttgattttgttgcagttctgaccggcaccaccggtgcaaatgaattttctgaatataatccgggtgccgtgattgaagatctgagtggtctgctggatatgtttatgctgtaa

SEQ ID NO: 47 - DNA, 352 bp, synthetic construct
  source 1..352: mol_type = other DNA
atgatcaagtataaagccgtgtttttcgacttcgactatacgttggccgatagcagcaaagctgttattgagtgcatcaattatgcgctgcagaaaatgggttacccggagagcagcccggagtctatttgtcgtaccattggcctgacgttggcagaggcattcaaaatcctgagcggcgataccagcgacagcaacgcagatctgtttcgtcaatactttaaggaacgtgcggatctggttatgtgtgaccgcaccgttatgtacagcaccgttgagtgcgtcctgaagaaactgaaaaaagccgatgttaagaccggtattgtctccacgaaataccgctaccgcatcg

SEQ ID NO: 48 - DNA, 657 bp, synthetic construct
  source 1..657: mol_type = other DNA
atgatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaaaaagccgatgttaagaccggtattgtctccacgaaataccgctaccgcatcgaagatatcttaaagcgtcacaagctgctccagtattttgacgtcatcgtgggtggcgaagatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaggatctgtccggtctgctggatatgttcatgctgtgataa

SEQ ID NO: 49 - DNA, 657 bp, synthetic construct
  source 1..657: mol_type = other DNA
atgatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaaaaagccgatgttaagaccggtattgtctccacgaaataccgctaccgcatcgaagcgatcttaaagcgtcacaagctgctccagtattttgacgtcatcgtgggtggcgaagatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 50 - DNA, 657 bp, synthetic construct
  source 1..657: mol_type = other DNA
atgatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaaaaagccgatgttaagaccggtattgtctccacgaaataccgctaccgcatcgaagatatcttaaagcgtcacaagctgctccagtattttgacgtcatcatcggtggcgaagatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 51 - DNA, 657 bp, synthetic construct
  source 1..657: mol_type = other DNA
atgatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaaaaagccgatgttaagaccggtattgtctccacgaaataccgctaccgcatcgaagatatcttaaagcgtcacaagctgctccagtattttgacgtcatcgtgggtggcgacgatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 52 - DNA, 657 bp, synthetic construct
  source 1..657: mol_type = other DNA
atgatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaacaggccgatgttaagaccggtattgtctccacgaaataccgctaccgcatcgaagcgatcttaaagcgtcacaagctgctccagtattttgacgtcatcgtgggtggcgaagatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 53 - DNA, 657 bp, synthetic construct
  source 1..657: mol_type = other DNA
atgatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaaaaagccgatgttaagaccggtattgtctccacgaaataccgctaccgcatcgaagcgatcttaaagcgtcacaagctgctccagtattttgacgtcatcatcggtggcgaagatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 54 - DNA, 657 bp, synthetic construct
  source 1..657: mol_type = other DNA
atgatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaaaaagccgatgttaagaccggtattgtctccacgaaataccgctaccgcatcgaagcgatcttaaagcgtcacaagctgctccagtattttgacgtcatcgtgggtggcgacgatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 55 - DNA, 657 bp, synthetic construct
  source 1..657: mol_type = other DNA
atgatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaacaggccgatgttaagaccggtattgtctccacgaaataccgctaccgcatcgaagatatcttaaagcgtcacaagctgctccagtattttgacgtcatcatcggtggcgaagatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 56 - DNA, 657 bp, synthetic construct
  source 1..657: mol_type = other DNA
atgatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaacaggccgatgttaagaccggtattgtctccacgaaataccgctaccgcatcgaagatatcttaaagcgtcacaagctgctccagtattttgacgtcatcgtgggtggcgacgatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 57 - DNA, 657 bp, synthetic construct
  source 1..657: mol_type = other DNA
atgatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaaaaagccgatgttaagaccggtattgtctccacgaaataccgctaccgcatcgaagatatcttaaagcgtcacaagctgctccagtattttgacgtcatcatcggtggcgacgatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 58 - DNA, 657 bp, synthetic construct
  source 1..657: mol_type = other DNA
atgatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaacaggccgatgttaagaccggtattgtctccacgaaataccgctaccgcatcgaagcgatcttaaagcgtcacaagctgctccagtattttgacgtcatcatcggtggcgaagatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 59 - DNA, 657 bp, synthetic construct
  source 1..657: mol_type = other DNA
atgatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaacaggccgatgttaagaccggtattgtctccacgaaataccgctaccgcatcgaagcgatcttaaagcgtcacaagctgctccagtattttgacgtcatcgtgggtggcgacgatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 60 - DNA, 657 bp, synthetic construct
  source 1..657: mol_type = other DNA
atgatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaaaaagccgatgttaagaccggtattgtctccacgaaataccgctaccgcatcgaagcgatcttaaagcgtcacaagctgctccagtattttgacgtcatcatcggtggcgacgatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 61 - DNA, 657 bp, synthetic construct
  source 1..657: mol_type = other DNA
atgatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaacaggccgatgttaagaccggtattgtctccacgaaataccgctaccgcatcgaagatatcttaaagcgtcacaagctgctccagtattttgacgtcatcatcggtggcgacgatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 62 - DNA, 657 bp, synthetic construct
  source 1..657: mol_type = other DNA
atgatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaacaggccgatgttaagaccggtattgtctccacgaaataccgctaccgcatcgaagcgatcttaaagcgtcacaagctgctccagtattttgacgtcatcatcggtggcgacgatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 63 - DNA, 657 bp, synthetic construct
  source 1..657: mol_type = other DNA
atgatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacgcgaacgcagatctgtttgcgcaatactttaaggaacgtgcggatctggttatgtgtgaccgcaccgttatgttcagcaccgttgagtgcgtcctgaagaaactgaaacaggccgatgttaagaccggtattgtctccacgaaataccgctaccgcatcgaagatatcttaaagcgtcacaagctgctccagtattttgacgtcatcgtgggtggcgacgatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaggatctgtccggtctgctggatatgttcatgctgtgataa

SEQ ID NO: 64 - DNA, 657 bp, synthetic construct
  source 1..657: mol_type = other DNA
atgatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacgcgaacgcagatctgtttgcgcaatactttaaggaacgtgcggatctggttatgtgtgaccgcaccgttatgttcagcaccgttgagtgcgtcctgaagaaactgaaacaggccgatgttaagaccggtattgtctccacgaaataccgctaccgcatcgaagcgatcttaaagcgtcacaagctgctccagtattttgacgtcatcgtgggtggcgacgatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 65 - DNA, 657 bp, synthetic construct
  source 1..657: mol_type = other DNA
atgatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacgcgaacgcagatctgtttgcgcaatactttaaggaacgtgcggatctggttatgtgtgaccgcaccgttatgttcagcaccgttgagtgcgtcctgaagaaactgaaacaggccgatgttaagaccggtattgtctccacgaaataccgctaccgcatcgaagatatcttaaagcgtcacaagctgctccagtattttgacgtcatcatcggtggcgacgatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 66 - DNA, 657 bp, synthetic construct
  source 1..657: mol_type = other DNA
atgatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacgcgaacgcagatctgtttgcgcaatactttaaggaacgtgcggatctggttatgtgtgaccgcaccgttatgttcagcaccgttgagtgcgtcctgaagaaactgaaacaggccgatgttaagaccggtattgtctccacgaaataccgctaccgcatcgaagcgatcttaaagcgtcacaagctgctccagtattttgacgtcatcatcggtggcgacgatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 67 - DNA, 654 bp, synthetic construct
  source 1..654: mol_type = other DNA
atgatcaagtataaagccgtgtttttcgacttcgactatacgttggccgatagcagcaaagctgttattgagtgcatcaattatgcgctgcagaaaatgggttacccggagtgcagcccggactctatttgtcgtaccattggcctgagcttggcagaggcattcaaaatcctgaccggcgataccagcgacagcaacgcagatctgtttcgtcaatactttaaggaacgtgcggatctggttatgtgtgaccgcaccgttatgtacagcaccgttgagtgcgtcctgaagaaactgaaaaaagccgatgttaagaccggtattgtctccacgaaataccgctaccgcatcgaagcgatcttaaagcgtcacaagctgctccagtattttgacgtcatcgtgggtggcgaagatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattccggatctgtccggtctgctggatatgttcatgctgtaa

SEQ ID NO: 68 - DNA, 654 bp, synthetic construct
  source 1..654: mol_type = other DNA
atgatcaagtataaagccgtgtttttcgacttcgactatacgttggccgatagcagcaaagctgttattgagtgcatcaattatgcgctgcagaaaatgggttacccggagtgcagcccggactctatttgtcgtaccattggcctgacgttggcagaggcattcaaaatcctgagcggcgataccagcgacagcaacgcagatctgtttcgtcaatactttaaggaacgtgcggatctggttatgtgtgaccgcaccgttatgtacagcaccgttgagtgcgtcctgaagaaactgaaaaaagccgatgttaagaccggtattgtctccacgaaataccgctaccgcatcgaagcgatcttaaagcgtcacaagctgctccagtattttgacgtcatcgtgggtggcgaagatgtgaccgcgccgaagccagacccggaaggcctgaacaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattcgcggagtacaacccgggtgcggtaattccggatctgtccggtctgctggatatgttcatgctgtaa

SEQ ID NO: 69 - DNA, 654 bp, synthetic construct
  source 1..654: mol_type = other DNA
atgatcaagtataaagccgtgtttttcgacttcgactatacgttggccgatagcagcaaagctgttattgagtgcatcaattatgcgctgcagaaaatgggttacccggagtgcagcccggactctatttgtcgtaccattggcctgacgttggcagaggcattcaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaaaaagccgatgttaagaccggtattgtctccacgaaataccgctaccgcatcgaagatatcttaaagcgtcacaagctgctccagtattttgacgtcatcgtgggtggcgaagatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattccggatctgtccggtctgctggatatgttcatgctgtaa

SEQ ID NO: 70 - DNA, 672 bp, Clostridium thermocellum; identical to SEQ ID NO: 46
  source 1..672: mol_type = genomic DNA

SEQ ID NO: 71 - DNA, 675 bp, synthetic construct
  source 1..675: mol_type = other DNA
atgcaccaccatcatcatcacatcaagtataaagccgtgtttttcgacttcgactatacgttggccgatagcagcaaagctgttattgagtgcatcaattatgcgctgcagaaaatgggttacccggagagcagcccggagtctatttgtcgtaccattggcctgacgttggcagaggcattcaaaatcctgagcggcgataccagcgacagcaacgcagatctgtttcgtcaatactttaaggaacgtgcggatctggttatgtgtgaccgcaccgttatgtacagcaccgttgagtgcgtcctgaagaaactgaaaaaagccgatgttaagaccggtattgtctccacgaaataccgctaccgcatcgaagatatcttaaagcgtgacaagctgctccagtattttgacgtcatcgtgggtggcgaagatgtggcggcgcacaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 72 - DNA, 675 bp, synthetic construct
  source 1..675: mol_type = other DNA
atgcaccaccatcatcatcacatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaaaaagccgatgttaagaccggtattgtctccacgaaataccgctaccgcatcgaagatatcttaaagcgtcacaagctgctccagtattttgacgtcatcgtgggtggcgaagatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaggatctgtccggtctgctggatatgttcatgctgtgataa

SEQ ID NO: 73 - DNA, 675 bp, synthetic construct
  source 1..675: mol_type = other DNA
atgcaccaccatcatcatcacatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaaaaagccgatgttaagaccggtattgtctccacgaaataccgctaccgcatcgaagcgatcttaaagcgtcacaagctgctccagtattttgacgtcatcgtgggtggcgaagatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 74 - DNA, 675 bp, synthetic construct
  source 1..675: mol_type = other DNA
atgcaccaccatcatcatcacatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaaaaagccgatgttaagaccggtattgtctccacgaaataccgctaccgcatcgaagatatcttaaagcgtcacaagctgctccagtattttgacgtcatcatcggtggcgaagatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 75 - DNA, 675 bp, synthetic construct
  source 1..675: mol_type = other DNA
atgcaccaccatcatcatcacatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaaaaagccgatgttaagaccggtattgtctccacgaaataccgctaccgcatcgaagatatcttaaagcgtcacaagctgctccagtattttgacgtcatcgtgggtggcgacgatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 76 - DNA, 675 bp, synthetic construct
  source 1..675: mol_type = other DNA
atgcaccaccatcatcatcacatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaacaggccgatgttaagaccggtattgtctccacgaaataccgctaccgcatcgaagcgatcttaaagcgtcacaagctgctccagtattttgacgtcatcgtgggtggcgaagatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 77 - DNA, 675 bp, synthetic construct
  source 1..675: mol_type = other DNA
atgcaccaccatcatcatcacatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaaaaagccgatgttaagaccggtattgtctccacgaaataccgctaccgcatcgaagcgatcttaaagcgtcacaagctgctccagtattttgacgtcatcatcggtggcgaagatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 78 - DNA, 675 bp, synthetic construct
  source 1..675: mol_type = other DNA
atgcaccaccatcatcatcacatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaaaaagccgatgttaagaccggtattgtctccacgaaataccgctaccgcatcgaagcgatcttaaagcgtcacaagctgctccagtattttgacgtcatcgtgggtggcgacgatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 79 - DNA, 675 bp, synthetic construct
  source 1..675: mol_type = other DNA
atgcaccaccatcatcatcacatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaacaggccgatgttaagaccggtattgtctccacgaaataccgctaccgcatcgaagatatcttaaagcgtcacaagctgctccagtattttgacgtcatcatcggtggcgaagatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 80 - DNA, 675 bp, synthetic construct
  source 1..675: mol_type = other DNA
atgcaccaccatcatcatcacatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaacaggccgatgttaagaccggtattgtctccacgaaataccgctaccgcatcgaagatatcttaaagcgtcacaagctgctccagtattttgacgtcatcgtgggtggcgacgatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 81 - DNA, 675 bp, synthetic construct
  source 1..675: mol_type = other DNA
atgcaccaccatcatcatcacatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaaaaagccgatgttaagaccggtattgtctccacgaaataccgctaccgcatcgaagatatcttaaagcgtcacaagctgctccagtattttgacgtcatcatcggtggcgacgatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 82 - DNA, 675 bp, synthetic construct
  source 1..675: mol_type = other DNA
atgcaccaccatcatcatcacatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaacaggccgatgttaagaccggtattgtctccacgaaataccgctaccgcatcgaagcgatcttaaagcgtcacaagctgctccagtattttgacgtcatcatcggtggcgaagatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 83 - DNA, 675 bp, synthetic construct
  source 1..675: mol_type = other DNA
atgcaccaccatcatcatcacatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaacaggccgatgttaagaccggtattgtctccacgaaataccgctaccgcatcgaagcgatcttaaagcgtcacaagctgctccagtattttgacgtcatcgtgggtggcgacgatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 84 - DNA, 675 bp, synthetic construct
  source 1..675: mol_type = other DNA
atgcaccaccatcatcatcacatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaaaaagccgatgttaagaccggtattgtctccacgaaataccgctaccgcatcgaagcgatcttaaagcgtcacaagctgctccagtattttgacgtcatcatcggtggcgacgatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 85 - DNA, 675 bp, synthetic construct
  source 1..675: mol_type = other DNA
atgcaccaccatcatcatcacatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaacaggccgatgttaagaccggtattgtctccacgaaataccgctaccgcatcgaagatatcttaaagcgtcacaagctgctccagtattttgacgtcatcatcggtggcgacgatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 86 - DNA, 675 bp, synthetic construct
  source 1..675: mol_type = other DNA
atgcaccaccatcatcatcacatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacagcaacgcagatctgtttgcgcaatactttaaggaacgtgcggatctggttatgtgtgaccgcaccgttatgttcagcaccgttgagtgcgtcctgaagaaactgaaacaggccgatgttaagaccggtattgtctccacgaaataccgctaccgcatcgaagcgatcttaaagcgtcacaagctgctccagtattttgacgtcatcatcggtggcgacgatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 87 - DNA, 675 bp, synthetic construct
  source 1..675: mol_type = other DNA
atgcaccaccatcatcatcacatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacgcgaacgcagatctgtttgcgcaatactttaaggaacgtgcggatctggttatgtgtgaccgcaccgttatgttcagcaccgttgagtgcgtcctgaagaaactgaaacaggccgatgttaagaccggtattgtctccacgaaataccgctaccgcatcgaagatatcttaaagcgtcacaagctgctccagtattttgacgtcatcgtgggtggcgacgatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaggatctgtccggtctgctggatatgttcatgctgtgataa

SEQ ID NO: 88 - DNA, 675 bp, synthetic construct
  source 1..675: mol_type = other DNA
atgcaccaccatcatcatcacatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacgcgaacgcagatctgtttgcgcaatactttaaggaacgtgcggatctggttatgtgtgaccgcaccgttatgttcagcaccgttgagtgcgtcctgaagaaactgaaacaggccgatgttaagaccggtattgtctccacgaaataccgctaccgcatcgaagcgatcttaaagcgtcacaagctgctccagtattttgacgtcatcgtgggtggcgacgatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 89 - DNA, 675 bp, synthetic construct
  source 1..675: mol_type = other DNA
atgcaccaccatcatcatcacatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacgcgaacgcagatctgtttgcgcaatactttaaggaacgtgcggatctggttatgtgtgaccgcaccgttatgttcagcaccgttgagtgcgtcctgaagaaactgaaacaggccgatgttaagaccggtattgtctccacgaaataccgctaccgcatcgaagatatcttaaagcgtcacaagctgctccagtattttgacgtcatcatcggtggcgacgatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 90 - DNA, 675 bp, synthetic construct
  source 1..675: mol_type = other DNA
atgcaccaccatcatcatcacatcaagtataaagccgtgtttttcgacttcgactatacgttggccgatagcagcaaagctgttattgagtgcatcaattatgcgctgcagaaaatgggttacccggagagcagcccggactctatttgtcgtaccattggcctgacgttggcagaggcatacaaaatcctgaccggcgataccagcgacgcgaacgcagatctgtttgcgcaatactttaaggaacgtgcggatctggttatgtgtgaccgcaccgttatgttcagcaccgttgagtgcgtcctgaagaaactgaaacaggccgatgttaagaccggtattgtctccacgaaataccgctaccgcatcgaagcgatcttaaagcgtcacaagctgctccagtattttgacgtcatcatcggtggcgacgatgtgaccgcgccgaagccagacccggaaggcctgctgaaggcaattagcatggtcggttgccaaaaggaagaggttctgttcgttggtgacagcactgtggacgcgcgtacggcgaaaaacgctggcgtggactttgtggcagtcttgaccggcaccaccggtgcgaatgaattctcggagtacaacccgggtgcggtaattgaagatctgtccggtctgctggatatgttcatgctgtgataa

SEQ ID NO: 91 - protein, 6 aa, synthetic construct
HHHHHH

SEQ ID NO: 92 - protein, 7 aa, synthetic construct
DYKDDDK

(19) United States
(12) Patent Application Publication
(10) Pub. No.: US 2026/0167947 A1
(43) Pub. Date: Jun. 18, 2026

(54) COMPOSITIONS AND METHODS FOR THE PRODUCTION OF ALLULOSE

(71) Applicant: GreenLight Biosciences, Inc., Lexington, MA (US)

(72) Inventors: Matthew Eduardo Moura, Medford, MA (US); Andrew Simon, Medford, MA (US); Shreya Sawant, Medford, MA (US); Mark Welch, Newark, CA (US); Sridhar Govindarajan, Newark, CA (US)

(73) Assignee: GreenLight Biosciences, Inc., Medford, MA (US)

(21) Appl. No.: 18/711,064

(22) PCT Filed: Nov. 18, 2022
(86) PCT No.: PCT/US2022/080143

§ 371 Date: May 16, 2024

Related U.S. Application Data
(60) Provisional application No. 63/281,559, filed on Nov. 19, 2021.

(51) Int. Cl. C12N 9/16; C12N 9/10; C12N 9/92; C12P 19/02

(52) U.S. Cl. CPC C12N 9/16; C12N 9/1051; C12N 9/92; C12P 19/02; C12Y 204/01049; C12Y 301/03; C12Y 501/03; C12Y 503/01009; C12Y 504/02002

(57) ABSTRACT

Provided herein, in some embodiments, are allulose 6-phosphate phosphatases having improved properties (e.g., increased thermostability) and methods of using same for converting allulose-6-phosphate to allulose.

### RELATED APPLICATION

This application claims priority under 35 U.S.C. § 119(e) to U.S. provisional application, U.S. Ser. No. 63/281,559, filed Nov. 19, 2021, which is incorporated herein by reference in its entirety.

### REFERENCE TO AN ELECTRONIC SEQUENCE LISTING

The contents of the electronic sequence listing (G083070041WO00-SEQ-AZW.xml; Size: 119,803 bytes; and Date of Creation: Nov. 17, 2022) is herein incorporated by reference in its entirety.

### BACKGROUND OF THE INVENTION

Existing technologies for the conversion of polysaccharides to simple sugars employ multiple biotransformations, with extensive purification processes following each step. While these processes are relatively inexpensive, owing to the application of immobilized enzymes and continuous production systems, the downstream processing dramatically impacts cost.

### SUMMARY OF THE INVENTION

Provided herein, in some aspects, are mutant allulose 6-phosphate phosphatases (A6PPs) and methods of using same for converting allulose-6-phosphate to allulose (e.g., in vitro or cell-free methods). The mutant A6PPs of the disclosure have improved properties relative to wild-type A6PPs. In some embodiments, the mutant A6PPs of the disclosure have increased thermostability, improved selectivity for allulose-6-phosphate relative to alternative substrates, and increased activity (e.g., specific activity).

In some embodiments, increased thermostability of the A6PPs (1) enables thermal inactivation of deleterious activities contained within cellular lysates in which allulose is produced using the A6PPs, (2) decreases the chances of microbial contamination negatively impacting production runs, and/or (3) increases the half-life of the A6PP. In some embodiments, the mutant A6PPs of the disclosure are functional between 41° C. and 122° C. In some embodiments, the mutant A6PPs of the disclosure demonstrate improved thermostability and an increased half-life relative to the wild type.

Thus, some aspects of the present disclosure provide a mutant allulose 6-phosphate phosphatase (A6PP). In some embodiments, a mutant A6PP comprises an amino acid sequence at least 85% identical to SEQ ID NO: 1 and further comprises one or more amino acid mutations at positions selected from the group consisting of positions 41, 59, 89, 124, 140, and 142 of SEQ ID NO: 1. In some embodiments, a mutant A6PP comprises 1, 2, 3, 4, 5, or 6 amino acid mutations at positions selected from the group consisting of positions 41, 59, 89, 124, 140, and 142 of SEQ ID NO: 1. In some embodiments, the one or more A6PP amino acid mutation are selected from the group consisting of: (i) an aspartic acid (D) substitution at amino acid position 41 of SEQ ID NO: 1: (ii) a threonine (T) substitution at amino acid position 59 of SEQ ID NO: 1; (iii) a phenylalanine (F) substitution at amino acid position 89 of SEQ ID NO: 1; (iv) a histidine (H) substitution at amino acid position 124 of SEQ ID NO: 1; (v) a threonine (T) substitution at amino acid position 140 of SEQ ID NO: 1; and (vi) a proline (P) substitution at amino acid position 142 of SEQ ID NO: 1.

In some embodiments, the one or more A6PP amino acid mutations comprise each of: (i) an aspartic acid (D) substitution at amino acid position 41 of SEQ ID NO: 1; (ii) a threonine (T) substitution at amino acid position 59 of SEQ ID NO: 1; (iii) a phenylalanine (F) substitution at amino acid position 89 of SEQ ID NO: 1; (iv) a histidine (H) substitution at amino acid position 124 of SEQ ID NO: 1; (v) a threonine (T) substitution at amino acid position 140 of SEQ ID NO: 1; and (vi) a proline (P) substitution at amino acid position 142 of SEQ ID NO: 1. In some embodiments, the one or more A6PP amino acid mutations are selected from the group consisting of: E41D, S59T, Y89F, D124H, A140T, and H142P. In some embodiments, the one or more A6PP amino acid mutations comprise each of: E41D, S59T, Y89F, D124H, A140T, and H142P.

In some embodiments, the A6PP comprises (i) at least one amino acid mutation at position 41, 59, 89, 124, 140, and/or 142 (relative to the amino acid numbering of SEQ ID NO: 1) and (ii) at least 90% identical to SEQ ID NO: 1. In some embodiments, the A6PP comprises (i) at least one amino acid mutation at position 41, 59, 89, 124, 140, and/or 142 (relative to the amino acid numbering of SEQ ID NO: 1) and (ii) at least 95% identical to SEQ ID NO: 1. In some embodiments, the mutant A6PP is at least 85% identical to any one of SEQ ID NOs: 2-24.

Some aspects of the disclosure provide a mutant allulose 6-phosphate phosphatase (A6PP) comprising an amino acid sequence at least 85% identical to SEQ ID NO: 1 and further comprising one or more amino acid mutations at positions selected from the group consisting of positions 38, 41, 124, 140, 142 and 206 of SEQ ID NO: 1.

In some embodiments, the one or more amino acid mutations are selected from the group consisting of: (i) a cysteine (C) substitution at amino acid position 38 of SEQ ID NO: 1; (ii) an aspartic acid (D) substitution at amino acid position 41 of SEQ ID NO: 1; (iii) a histidine (H) substitution at amino acid position 124 of SEQ ID NO: 1; (iv) a threonine (T) substitution at amino acid position 140 of SEQ ID NO: 1; (v) a proline (P) substitution at amino acid position 142 of SEQ ID NO: 1; and (vi) a proline (P) substitution at amino acid position 206 of SEQ ID NO: 1. In some embodiments, the one or more amino acid mutations comprise each of the following: (i) a cysteine (C) substitution at amino acid position 38 of SEQ ID NO: 1; (ii) an aspartic acid (D) substitution at amino acid position 41 of SEQ ID NO: 1; (iii) a histidine (H) substitution at amino acid position 124 of SEQ ID NO: 1; (iv) a threonine (T) substitution at amino acid position 140 of SEQ ID NO: 1; (v) a proline (P) substitution at amino acid position 142 of SEQ ID NO: 1; and (vi) a proline (P) substitution at amino acid position 206 of SEQ ID NO: 1.

In some embodiments, the one or more A6PP amino acid mutations are selected from the group consisting of: S38C, E41D, D124H, A140T, H142P, and E206P. In some embodiments, the one or more amino acid mutations comprise: S38C, E41D, D124H, A140T, H142P, and E206P.

In some embodiments, the A6PP comprises (i) at least one amino acid mutation at position 38, 41, 124, 140, 142, and/or 206 (relative to the amino acid numbering of SEQ ID NO: 1) and (ii) at least 90% identical to SEQ ID NO: 1. In some embodiments, the A6PP comprises (i) at least one amino acid mutation at position 38, 41, 124, 140, 142, and/or 206 (relative to the amino acid numbering of SEQ ID NO: 1) and (ii) at least 95% identical to SEQ ID NO: 1.

In some embodiments, the A6PP further comprises one or more additional amino acid mutations. In some embodiments, the one or more additional amino acid mutations at positions selected from the group consisting of: positions 50, 59, 71, 119, 150, and 197 of SEQ ID NO: 1. In some embodiments, the one or more additional amino acid mutations are selected from the group consisting of: (a) a serine (S) substitution at amino acid position 50 of SEQ ID NO: 1; (b) a threonine (T) substitution at amino acid position 59 of SEQ ID NO: 1; (c) an alanine (A) substitution at amino acid position 71 of SEQ ID NO: 1; (d) an alanine (A) substitution at amino acid position 119 of SEQ ID NO: 1; (e) an asparagine (N) substitution at amino acid position 150 of SEQ ID NO: 1; and (f) an alanine (A) substitution at amino acid position 197 of SEQ ID NO: 1. In some embodiments, the one or more additional amino acid mutations are selected from the group consisting of T50S, S59T, R71A, D119A, L150N, and S197A.

Some aspects of the disclosure provide a mutant A6PP comprising an allulose 6-phosphate phosphatase (A6PP), wherein the A6PP comprises the amino acid sequence of any one of SEQ ID NOs: 2-24. In some embodiments, the disclosure provides a mutant A6PP comprising an allulose 6-phosphate phosphatase (A6PP), wherein the A6PP does not consist of the amino acid sequence of SEQ ID NO: 1, and wherein the A6PP comprises an amino acid sequence having at least 90%, at least 95%, at least 97%, at least 98%, or at least 99% identity to any one of SEQ ID NOs: 2-24. In some embodiments, the disclosure provides a mutant A6PP comprising an allulose 6-phosphate phosphatase (A6PP), wherein the A6PP does not consist of the amino acid sequence of SEQ ID NO: 1, and wherein the A6PP comprises an amino acid sequence having no more than comprises 1, 2, 3, 4, 5, or 6 amino acid mutations relative to any one of SEQ ID NOs: 2-24.

In some embodiments, the A6PP further comprises one or more additional amino acid mutations. In some embodiments, the one or more additional amino acid mutations at positions selected from the group consisting of positions 38, 55, 65, 71, 101, 103, 119, 134, 137, 197, 198, and 206 of SEQ ID NO: 1. In some embodiments, the one or more additional amino acid mutations are selected from the group consisting of: (a) a cysteine (C) substitution at amino acid position 38 of SEQ ID NO: 1; (b) a tyrosine (Y) substitution at amino acid position 55 of SEQ ID NO: 1; (c) an alanine (A) substitution at amino acid position 65 of SEQ ID NO: 1; (d) an alanine (A) substitution at amino acid position 71 of SEQ ID NO: 1; (e) a glutamine (Q) substitution at amino acid position 101 of SEQ ID NO: 1; (f) a glycine (G) substitution at amino acid position 103 of SEQ ID NO: 1; (g) an alanine (A) substitution at amino acid position 119 of SEQ ID NO: 1; (h) an isoleucine (I) substitution at amino acid position 134 of SEQ ID NO: 1; (i) an aspartic acid (D) substitution at amino acid position 137 of SEQ ID NO: 1; (j) an alanine (A) substitution at amino acid position 197 of SEQ ID NO: 1; (k) an alanine (A) substitution at amino acid position 198 of SEQ ID NO: 1; and (1) a proline (P) substitution at amino acid position 206 of SEQ ID NO: 1.

In some embodiments, the one or more additional amino acid mutations are selected from the group consisting of S38C, F55Y, S65A, R71A, K101Q, D103G, D119A, V134I, E137D, S197A, E198A, and E206P.

In some embodiments, the mutant A6PP has a half-life of about two hours or more at about 60° C. In some embodiments, the mutant A6PP has a longer half-life than an A6PP having the amino acid sequence of SEQ ID NO: 1. In some embodiments, the mutant A6PP is more selective for A6P relative to fructose 6-phosphate and/or glucose 6-phosphate than an A6PP having the amino acid sequence of SEQ ID NO: 1.

Some aspects of the disclosure provide a nucleic acid encoding a mutant A6PP as described herein (e.g., a mutant A6PP comprising the amino acid sequence of any one of SEQ ID NOs: 2-24). Some aspects of the disclosure provide a nucleic acid comprising a nucleotide sequence at least 85% identical to any one of SEQ ID NO: 47-69.

Some aspects of the disclosure provide a method of producing allulose comprising: converting allulose 6-phosphate (A6P) to allulose catalyzed by a mutant A6PP as described herein (e.g., a mutant A6PP comprising the amino acid sequence of any one of SEQ ID NOs: 2-24). Some aspects of the disclosure provide a method for producing allulose comprising: converting allulose-6-phosphate (A6P) to allulose catalyzed using an allulose 6-phosphate phosphatase (A6PP), wherein the A6PP is encoded by a nucleic acid comprising a nucleotide sequence at least 85% identical (e.g., at least 90%, 95%, 97%, 98%, 99%, or 100% identical) to any one of SEQ ID NO: 47-69.

In some embodiments, the nucleic acid is expressed in a microbial cell.

In some embodiments, the method further comprises converting fructose 6-phosphate (F6P) to allulose 6-phosphate (A6P) using an allulose 6-phosphate epimerase (A6PE). In some embodiments, the method further comprises converting glucose 6-phosphate (G6P) to fructose 6-phosphate (F6P) using a phosphoglucoisomerase. In some embdoiments, the method further comprises converting glucose 1-phosphate (G1P) to produce glucose 6-phosphate (G6P) using a phosphoglucomutase. In some embodiments, the method further comprises converting a polymeric glucose carbohydrate to glucose 1-phosphate (G1P) using an α-glucan or a cellodextrin phosphorylase.

Some aspects of the disclosure provide a cell comprising a mutant A6PP described herein (e.g., a mutant A6PP comprising the amino acid sequence of any one of SEQ ID NOs: 2-24; or a nucleic acid comprising a nucleotide sequence at least 85% identical to any one of SEQ ID NO: 47-69).

Some aspects of the disclosure provide a cell lysate comprising a mutant A6PP described herein (e.g., a mutant A6PP comprising the amino acid sequence of any one of SEQ ID NOs: 2-24; or a nucleic acid comprising a nucleotide sequence at least 85% identical to any one of SEQ ID NO: 47-69).

Some aspects of the disclosure provide a kit comprising: (i) a mutant A6PP described herein (e.g., a mutant A6PP comprising the amino acid sequence of any one of SEQ ID NOs: 2-24); and (ii) a reaction buffer. Some aspects of the disclosure provide a kit comprising: (i) a nucleic acid encoding a mutant A6PP described herein (e.g., a nucleic acid comprising a nucleotide sequence at least 85%, 90%, 95%, or 100% identical to any one of SEQ ID NO: 47-69); and (ii) a reaction buffer.

The details of one or more embodiments of the invention are set forth herein. Other features, objects, and advantages of the invention will be apparent from the Detailed Description, the Examples, and the Claims.

### BRIEF DESCRIPTION OF THE DRAWINGS

FIGS. 1A-1B are graphs showing the specificity of selected A6PPs for allulose-6-phosphate (A6P) as an enzymatic substrate compared to fructose-6-phosphate (F6P) or glucose-6-phosphate (G6P).

FIGS. 2A-2B are graphs showing the residual activity of selected A6PPs to utilize A6P as an enzymatic substrate following pretreatment of the protein at the indicated temperature for 60 minutes.

FIG. 3 provides a graphical representation of the impact of specific A6PP substitutions on thermostability, specificity, specific activity, and protein expression.

### DETAILED DESCRIPTION

The present disclosure provides, in some embodiments, highly efficient and cost-effective methods, compositions of mutant allulose 6-phosphate phosphatases, and systems for producing allulose (e.g., from allulose 6-phosphate). These methods, compositions, and systems for producing allulose are highly-efficient and cost-effective due to the improved properties (e.g., increased thermostability) of the mutant A6PPs of the disclosure.

In some embodiments, provided herein are mutant allulose 6-phosphate phosphatases (A6PPs). The mutant A6PPs of the disclosure enzymatically convert allulose 6-phosphate (A6P) to allulose. In some embodiments, a mutant A6PP has a half-life that is longer than the half-life of a wild-type A6PP (e.g., a wild-type A6PP having the amino acid sequence of SEQ ID NO: 1). In some embodiments, a mutant A6PP has a half-life of more than two hours at 60° C. In some embodiments, a mutant A6PP has a half-life of more than 2, 3, 4, 5, 6, 7, 8, 9, or 10 hours at 60° C. In some embodiments, a mutant A6PP has a half-life of more than 2, 3, 4, 5, 6, 7, 8, 9, or 10 hours at temperatures of 60° C. or higher. In some embodiments, a mutant A6PP has a half-life of more than 2, 3, 4, 5, 6, 7, 8, 9, or 10 hours at temperatures ranging from 50° C. to 80° C., 50° C. to 70° C., 50° C. to 60° C., or 60° C. to 80° C. An increased half-life allows for increased yields of allulose in production methods (e.g., large-scale production methods). In some embodiments, a mutant A6PP has increased thermostability relative to a wild-type A6PP. In some embodiments, a mutant A6PP is capable of functioning at elevated temperatures (e.g., at or above 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., or 85° C.). In some embodiments, a mutant A6PP is capable of functioning at elevated temperatures (e.g., at or above 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., or 85° C.) for longer periods of time relative to a wild-type A6PP (e.g., a wild-type A6PP having the amino acid sequence of SEQ ID NO: 1). In some embodiments, a mutant A6PP is capable of functioning at elevated temperatures (e.g., at or above 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., or 85° C.) for 1, 2, 3, 4, 5, 6, 7, 8, 9, 10, 12, 15, 20, 30, 40, or 50 hours.

In some embodiments, a mutant A6PP has improved selectivity for allulose 6-phosphate as a substrate compared to any other possible substrates (e.g., fructose 6-phosphate or glucose 6-phosphate). In some embodiments, a mutant A6PP has at least 10%, 20%, 30%, 40%, 50%, 60%, 70%, 80%, 90%, or 100% improved selectivity for allulose 6-phosphate as a substrate compared to any other possible substrates (e.g., fructose 6-phosphate or glucose 6-phosphate). In some embodiments, a mutant A6PP has increased binding affinity for allulose 6-phosphate compared to other possible substrates (e.g., fructose 6-phosphate or glucose 6-phosphate). In some embodiments, a mutant A6PP has a binding affinity for allulose 6-phosphate that is at least two-fold, three-fold, four-fold, or five-fold higher than its binding affinity for other possible substrates (e.g., fructose 6-phosphate or glucose 6-phosphate). In some embodiments, a mutant A6PP has a binding affinity for allulose 6-phosphate that is at least 10%, 20%, 30%, 40%, 50%, 60%, 70%, 80%, 90%, or 100% higher than its binding affinity for other possible substrates (e.g., fructose 6-phosphate or glucose 6-phosphate).

In some embodiments, a mutant A6PP comprises an amino acid sequence comprising one or more amino acid mutations at positions selected from the group consisting of positions 41, 59, 89, 124, 140, and 142 of SEQ ID NO: 1. In some embodiments, a mutant A6PP comprises an aspartic acid (D) substitution at amino acid position 41 of SEQ ID NO: 1. In some embodiments, a mutant A6PP comprises an aspartic acid (D) substitution at amino acid position 41 and at least one additional amino acid mutation (e.g., an amino acid mutation at positions 59, 89, 124, 140, and 142 of SEQ ID NO: 1). In some embodiments, a mutant A6PP comprises a threonine (T) substitution at amino acid position 59 of SEQ ID NO: 1. In some embodiments, a mutant A6PP comprises a threonine (T) substitution at amino acid position 59 of SEQ ID NO: 1 and at least one additional amino acid mutation (e.g., an amino acid mutation at positions 41, 89, 124, 140, and 142 of SEQ ID NO: 1). In some embodiments, a mutant A6PP comprises a phenylalanine (F) substitution at amino acid position 89 of SEQ ID NO: 1. In some embodiments, a mutant A6PP comprises a phenylalanine (F) substitution at amino acid position 89 of SEQ ID NO: 1 and at least one additional amino acid mutation (e.g., an amino acid mutation at positions 41, 59, 124, 140, and 142 of SEQ ID NO: 1). In some embodiments, a mutant A6PP comprises a histidine (H) substitution at amino acid position 124 of SEQ ID NO: 1. In some embodiments, a mutant A6PP comprises a histidine (H) substitution at amino acid position 124 of SEQ ID NO: 1 and at least one additional amino acid mutation (e.g., an amino acid mutation at positions 41, 59, 89, 140, and 142 of SEQ ID NO: 1). In some embodiments, a mutant A6PP comprises a threonine (T) substitution at amino acid position 140 of SEQ ID NO: 1. In some embodiments, a mutant A6PP comprises a threonine (T) substitution at amino acid position 140 of SEQ ID NO: 1 and at least one additional amino acid mutation (e.g., an amino acid mutation at positions 41, 59, 89, 124, and 142 of SEQ ID NO: 1). In some embodiments, a mutant A6PP comprises a proline (P) substitution at amino acid position 142 of SEQ ID NO: 1. In some embodiments, a mutant A6PP comprises a proline (P) substitution at amino acid position 142 of SEQ ID NO: 1 and at least one additional amino acid mutation (e.g., an amino acid mutation at positions 41, 59, 89, 124, and 140 of SEQ ID NO: 1).

In some embodiments, a mutant A6PP comprises one or more amino acid mutations at positions selected from the group consisting of positions 38, 55, 65, 71, 101, 103, 119, 134, 137, 197, 198, and 206 of SEQ ID NO: 1. In some embodiments, a mutant A6PP comprises a cysteine (C) substitution at amino acid position 38 of SEQ ID NO: 1. In some embodiments, a mutant A6PP comprises a tyrosine (Y) substitution at amino acid position 55 of SEQ ID NO: 1. In some embodiments, a mutant A6PP comprises an alanine (A) substitution at amino acid position 65 of SEQ ID NO: 1. In some embodiments, a mutant A6PP comprises an alanine (A) substitution at amino acid position 71 of SEQ ID NO: 1. In some embodiments, a mutant A6PP comprises a glutamine (Q) substitution at amino acid position 101 of SEQ ID NO: 1. In some embodiments, a mutant A6PP comprises a glycine (G) substitution at amino acid position 103 of SEQ ID NO: 1. In some embodiments, a mutant A6PP comprises an alanine (A) substitution at amino acid position 119 of SEQ ID NO: 1. In some embodiments, a mutant A6PP comprises an isoleucine (I) substitution at amino acid position 134 of SEQ ID NO: 1. In some embodiments, a mutant A6PP comprises an aspartic acid (D) substitution at amino acid position 137 of SEQ ID NO: 1. In some embodiments, a mutant A6PP comprises an alanine (A) substitution at amino acid position 197 of SEQ ID NO: 1. In some embodiments, a mutant A6PP comprises an alanine (A) substitution at amino acid position 198 of SEQ ID NO: 1. In some embodiments, a mutant A6PP comprises a proline (P) substitution at amino acid position 206 of SEQ ID NO: 1.

In some embodiments, a mutant A6PP comprises S38C, F55Y, S65A, R71A, K101Q, D103G, D119A, V134I, E137D, S197A, E198A, and/or E206P.

In some embodiments, a mutant A6PP comprises substitutions at positions E41, S59, Y89, D124, A140, and H142 relative to SEQ ID NO: 1. In some embodiments, a mutant A6PP comprises E41D, S59T, Y89F, D124H, A140T, and H142P substitutions relative to SEQ ID NO: 1. In some embodiments, a mutant A6PP comprises the amino acid sequence of any one of SEQ ID NOs: 2-24. In some embodiments, the A6PP comprises (i) at least one amino acid mutation at position 41, 59, 89, 124, 140, and/or 142 (relative to the amino acid numbering of SEQ ID NO: 1) and (ii) at least 90% identical to SEQ ID NO: 1. In some embodiments, the A6PP comprises (i) at least one amino acid mutation at position 41, 59, 89, 124, 140, and/or 142 (relative to the amino acid numbering of SEQ ID NO: 1) and (ii) at least 95% identical to SEQ ID NO: 1. In some embodiments, the mutant A6PP is at least 85% identical to any one of SEQ ID NOs: 2-24.

In some embodiments, a mutant A6PP comprises substitutions at positions S38, E41, D124, A140, H142, and/or E206 relative to SEQ ID NO: 1. In some embodiments, a mutant A6PP comprises S38C, E41D, D124H, A140T, H142P, and/or E206P substitutions relative to SEQ ID NO: 1.

In some embodiments, a mutant A6PP further comprises a protein purification tag. In some embodiments, a protein purification tag is an N-terminal histidine tag (e.g., a HHHHHH (SEQ ID NO: 91) sequence) or a C-terminal histidine tag. In some embodiments, a protein purification tag is an N-terminal FLAG tag (e.g., a DYKDDDK (SEQ ID NO: 92) sequence) or a C-terminal FLAG tag. In some embodiments, a protein purification tag is an N-terminal hemagglutinin tag or a C-terminal hemagglutinin tag. In some embodiments, a mutant A6PP comprises the amino acid sequence of any one of SEQ ID NOs: 26-45.

In some embodiments, a mutant A6PP comprises one or more amino acid mutations (e.g., substitutions) relative to SEQ ID NO: 1. In some embodiments, a mutant A6PP comprises 1, 2, 3, 4, 5, 6, 7, 8, 9, 10, 11, 12, 13, 14, 15, 16, 17, 18, 19, 20, or more amino acid mutations (e.g., substitutions) relative to SEQ ID NO: 1. In some embodiments, a mutant A6PP comprises an amino acid sequence at least 70%, 75%, 80%, 85%, 90%, 95%, or 97% identical to SEQ ID NO: 1.

Throughout this disclosure, the numbering of amino acid positions of mutant A6PPs is described within SEQ ID NO: 1 (e.g., positions S38, E41, D124, A140, H142, E206 of SEQ ID NO: 1). It should be understood that description of amino acid position is performed relative to SEQ ID NO: 1 and not exclusively within the backbone of an amino acid sequence comprising or consisting of SEQ ID NO: 1. For example, it should be understood that addition of amino acids at the N-terminal of a protein will shift the relative numbering of the positions described herein. In embodiments comprising a N-terminal hexahistidine tag, for example, the amino acids corresponding to positions S38, E41, D124, A140, H142, and E206 of SEQ ID NO: 1 will be numbered as S44, E47, D130, A146, H148, and E212 (see, e.g., SEQ ID NO: 25). Similarly, deletion of amino acids may result in altered numbering. For example, deletion of the first ten amino acids of SEQ ID NO: 1 will result in the amino acids corresponding to positions S38, E41, D124, A140, H142, and E206 of SEQ ID NO: 1 to be numbered as S28, E31, D114, A130, H132, and E196.

### Allulose Production

Some aspects of the present disclosure provide methods, compositions, and systems for producing allulose. In some embodiments, the methods of the disclosure involve the use of a mutant allulose 6-phosphate phosphatase (A6PP) (e.g., a mutant A6PP comprising the amino acid sequence of any one of SEQ ID NOs: 2-24). In some embodiments, a mutant A6PP catalyzes an enzymatic conversion from allulose 6-phosphate to allulose.

These methods, in some embodiments, include culturing cells engineered to express at least one pullulanse or isoamlyase, at least one α-glucan phosphorylase, at least one phosphoglucomutase, at least one phosphoglucoisomerase, at least one allulose 6-phosphate epimerase, at least one mutant allulose 6-phosphate phosphatase (e.g., a mutant A6PP comprising the amino acid sequence of any one of SEQ ID NOs: 2-24), or a combination of at least two (e.g., at least three, or at least four) of the foregoing enzymes. These methods, in some embodiments, include culturing cells engineered to express at least one pullulanse or isoamlyase, at least one cellodextrin phosphorylase, at least one phosphoglucomutase, at least one phosphoglucoisomerase, at least one allulose 6-phosphate epimerase, at least one mutant allulose 6-phosphate phosphatase, or a combination of at least two (e.g., at least three, or at least four) of the foregoing enzymes.

In some embodiments, methods of producing allulose utilize any of the methods, enzymes, or compositions as described in International Patent Publication WO2018129275A1, published Jul. 12, 2018; or International Patent Publication WO2020132027A2, published Jun. 25, 2020.

Enzymes of the allulose production pathways as provided herein are typically heterologous to the host cell, although some of the enzymes may be endogenous (native) to the host cell. Thus, in some embodiments, at least one enzyme (e.g., thermostable enzyme) used to convert a polysaccharide to allulose is heterologous to the host cell. In some embodiments, at least two, at least three, or at least four enzymes are heterologous to the host cell. In some embodiments, at least one enzyme is endogenous (native) to the host cell. In some embodiments, at least two, at least three, or at least four enzymes are endogenous to the host cell. In some embodiments, the mutant A6PPs are heterologous to the host cell. The enzymes of the allulose production pathways described herein (e.g., the mutant A6PPs of the disclosure) may be produced by the host cell.

The host cells may be prokaryotic cells, such as bacterial cells (e.g., Escherichia coli cells), or eukaryotic cells, such as yeast cells or plant cells. Other cell types are described below.

In some embodiments, at least one of the enzymes used to convert a polysaccharide to allulose is a thermostable enzyme. In some embodiments, at least two (e.g., at least three or at least four) of the enzymes are thermostable enzymes. In some embodiments, all of the enzymes are thermostable enzymes. Thus, in some embodiments, the methods include culturing cells engineered to express at least one thermostable α-glucan phosphorylase, at least one thermostable phosphoglucomutase, at least one thermostable phosphoglucoisomerase, at least one thermostable allulose 6-phosphate epimerase, at least one thermostable allulose 6-phosphate phosphatase (e.g., an A6PP comprising the amino acid sequence of any one of SEQ ID NOs: 2-24), optionally at least one debranching enzyme, or a combination of at least two or more of the foregoing thermostable enzymes.

In some embodiments, at least one of the enzymes used to convert cellulose/cellodextrin to allulose is a thermostable enzyme. In some embodiments, at least two (e.g., at least three or at least four) of the enzymes are thermostable enzymes. In some embodiments, all of the enzymes are thermostable enzymes. Thus, in some embodiments, the methods include culturing cells engineered to express at least one thermostable cellodextrin phosphorylase, at least one thermostable phosphoglucomutase, at least one thermostable phosphoglucoisomerase, at least one thermostable allulose 6-phosphate epimerase, at least one thermostable allulose 6-phosphate phosphatase (e.g., a mutant A6PP comprising the amino acid sequence of any one of SEQ ID NOs: 2-24), optionally at least one debranching enzyme, or a combination of at least two or more of the foregoing thermostable enzymes.

In some embodiments, the methods of producing allulose include lysing (e.g., thermal, osmotic, mechanical, chemical, or enzymatic lysis) the cultured cells to produce at least one (e.g., at least two, at least three, or at least four) cell lysate. It should be understood that multiple cell lysates (and thus multiple cell populations, e.g., from the same organism (e.g., bacteria) or from different organisms (e.g., bacteria, yeast, and/or plant cells)) may be used in the production of allulose as provided herein. For example, one cell population may be engineered to express one or more enzymes(s) of the allulose production pathway, while another cell population (or several other cell populations) may be engineered to express another (at least one other) enzyme of the allulose production pathway. Thus, in some embodiments, the methods comprise culturing at least one population of cells engineered to express at least one α-glucan phosphorylase, culturing at least one cell population engineered to express at least one phosphoglucomutase, culturing at least one cell population engineered to express at least one phosphoglucoisomerase, culturing at least one cell population engineered to express at least one allulose 6-phosphate epimerase, culturing at least one cell population engineered to express at least one mutant allulose 6-phosphate phosphatase (e.g., a mutant A6PP comprising the amino acid sequence of any one of SEQ ID NOs: 2-24), and/or culturing at least one cell population engineered to express at least one debranching enzyme. Following lysis of the cells, the cell lysates are combined such that the enzymes are present in a single cell lysate/reaction mixture. Thus, in some embodiments, the methods comprise culturing at least one population of cells engineered to express at least one cellodextrin phosphorylase, culturing at least one cell population engineered to express at least one phosphoglucomutase, culturing at least one cell population engineered to express at least one phosphoglucoisomerase, culturing at least one cell population engineered to express at least one allulose 6-phosphate epimerase, culturing at least one cell population engineered to express at least one mutant allulose 6-phosphate phosphatase (e.g., a mutant A6PP comprising the amino acid sequence of any one of SEQ ID NOs: 2-24), and/or culturing at least one cell population engineered to express at least one debranching enzyme. Following lysis of the cells, the cell lysates are combined such that the enzymes are present in a single cell lysate/reaction mixture. In some embodiments, the enzymes (e.g., the mutant A6PPs of the disclosure) are purified or partially purified prior to use in a method of producing allulose.

Cell lysates in some embodiments, may be combined with a nutrient. For example, cell lysates may be combined with Na₂HPO₄, KH₂PO₄, NH₄Cl, NaCl, MgSO₄, and/or CaCl₂. Examples of other nutrients include, without limitation, magnesium sulfate, magnesium chloride, magnesium orotate, magnesium citrate, manganese chloride, calcium chloride, cobalt chloride, zinc chloride, zinc sulfate, potassium acetate, potassium phosphate monobasic, potassium phosphate dibasic, potassium phosphate tribasic, sodium phosphate monobasic, sodium acetate, sodium chloride, sodium phosphate dibasic, sodium phosphate tribasic, ammonium phosphate monobasic, ammonium phosphate dibasic, ammonium sulfate, ammonium chloride, and ammonium hydroxide.

Cell lysates, in some embodiments, may be combined with a cofactor. For example, cell lysates may be combined with adenosine diphosphate (ADP), adenosine triphosphate (ATP), nicotinamide adenine dinucleotide (NAD+), or other non-protein chemical compounds required for activity of an enzyme (e.g., inorganic ions and coenzymes).

Cell lysates in some embodiments, may be combined with a substrate. For example, cell lystates comprising a mutant A6PP may be combined with

It should be understood that in any one of the methods described herein, the cells may be lysed by any means, including mechanical, chemical, enzymatic, osmotic, and/or thermal lysis. Thus, in certain embodiments, a lysing step and a heating (heat inactivation) step may be combined as a single step of heating the cells to a temperature that lyses the cells and inactivates undesired native enzymatic activities.

In some embodiments, the methods further include heating the cell lysate(s) (or a cell lysate mixture) to a temperature that inactivates undesired native enzymatic activities but does not inactivate any of the thermostable enzymes of the production pathway, to produce a heat-inactivated lysate. The cell lysate(s), in some embodiments, is heated to a temperature of at least 50° C. For example, the cell lysate(s) may be heated to a temperature of at least 55° C., 60° C., 65° C., 70° C., 75° C., 80° C., 85° C., or 90° C. A native enzyme (or other non-thermostable enzyme) is considered inactive, in some embodiments, when its level of activity is reduced by at least 50%. In some embodiments, a native enzyme (or other non-thermostable enzyme) is considered inactive when its level of activity is reduced by at least 55%, 60%, 65%, 70%, 75%, 80%, 85%, 90%, 95%, 98%, 99%, or 100%.

The cell lysate(s) may be heated for a period of time sufficient to inactivate native enzymes (or other non-thermostable enzymes) of the cell. For example, the cell lysate(s) may be heated for at least 2, 3, 4, or 5 minutes. In some embodiments, the cell lysate(s) are heated for longer than 5 minutes. In some embodiments, the cell lysate(s) are heated for a period of time sufficient to reduce the activity of at least some of the native enzymes (or other non-thermostable enzymes) by at least 50% (e.g., at least 60%, 70%, 80%, or 90%).

Following heat inactivation, in some embodiments, at least one (e.g., at least two or at least three) purified enzyme (or partially purified enzyme) is added to the cell lysate/reaction mixture. Thus, a reaction mixture, in some embodiments, includes a combination of enzymes present in the cell lysate (expressed by the engineered host cell(s)), at least one cofactor or nutrient, and at least one purified enzyme. At least one purified enzyme may be selected from the group consisting of α-glucan phosphorylases, phosphoglucomutases, phosphoglucoisomerases, allulose 6-phosphate epimerases, allulose 6-phosphate phosphatases (e.g., a mutant A6PP comprising the amino acid sequence of any one of SEQ ID NOs: 2-24), and debranching enzymes. At least one purified enzyme may be selected from the group consisting of cellodextrin phosphorylases, phosphoglucomutases, phosphoglucoisomerases, allulose 6-phosphate epimerases, allulose 6-phosphate phosphatases (e.g., a mutant A6PP comprising the amino acid sequence of any one of SEQ ID NOs: 2-24), and debranching enzymes. Advantageously, this allows for the incorporation of a purified enzyme that is not part of a cell lysate and which may be commercially obtained, thus, alleviating the need to engineer a cell to express the needed enzyme.

In some embodiments, the methods also include incubating the heat-inactivated lysate(s) in the presence of a polysaccharide and inorganic phosphate to produce allulose. In some embodiments, the heat-inactivated lysates are incubated at a temperature of at least 50° C. In some embodiments, the heat-inactivated lysates are incubated for at least 2 minutes (e.g., at least 3, 4, or 5 minutes). For example, the heat-inactivated lysates may be incubated for 30-60 minutes, with optimized time reaching below 30 minutes, such as 25-30 minutes, 20-25 minutes, 15-20 minutes, 10-15 minutes, 5-10 minutes, 2-5 minutes, or 2-10 minutes. The polysaccharide may be, for example, a starch, cellulose, maltodextrin, and/or cellodextrin. In some embodiments, biomass is used instead of a polysaccharide. In some embodiments, the polysaccharide is maltodextrin and is present as a component of a compound (e.g., part of the biomass). For example, in some embodiments, the heat-inactivated lysate(s) (e.g., microbial cell lysates) are incubated in the presence of corn pulp and inorganic phosphate to produce allulose (or any other sugar described herein).

Also provided herein are cells and cell lysates used for the production of allulose. Thus, an engineered cell (e.g., bacterial cell, yeast cell, and/or plant cell) or cell lysate(s) of the present disclosure may include at least one (e.g., at least two, at least three, or at least four) enzyme selected from the group consisting of α-glucan phosphorylases, phosphoglucomutases, phosphoglucoisomerases, allulose 6-phosphate epimerases, allulose 6-phosphate phosphatases (e.g., a mutant A6PP comprising the amino acid sequence of any one of SEQ ID NOs: 2-24), and debranching enzymes. In some embodiments, an engineered cell (e.g., bacterial cell, yeast cell, and/or plant cell) or cell lysate(s) of the present disclosure includes at least one (e.g., at least two, at least three, or at least four) enzyme selected from the group consisting of thermostable α-glucan phosphorylases, thermostable phosphoglucomutases, thermostable phosphoglucoisomerases, thermostable allulose 6-phosphate epimerases, thermostable allulose 6-phosphate phosphatases, and thermostable debranching enzymes. Thus, an engineered cell (e.g., bacterial cell, yeast cell, and/or plant cell) or cell lysate(s) of the present disclosure may include at least one (e.g., at least two, at least three, or at least four) enzyme selected from the group consisting of cellodextrin phosphorylases, phosphoglucomutases, phosphoglucoisomerases, allulose 6-phosphate epimerases, allulose 6-phosphate phosphatases, and debranching enzymes. In some embodiments, an engineered cell (e.g., bacterial cell, yeast cell, and/or plant cell) or cell lysate(s) of the present disclosure includes at least one (e.g., at least two, at least three, or at least four) enzyme selected from the group consisting of thermostable cellodextrin phosphorylases, thermostable phosphoglucomutases, thermostable phosphoglucoisomerases, thermostable allulose 6-phosphate epimerases, thermostable allulose 6-phosphate phosphatases, and thermostable debranching enzymes.

Non-limiting examples of enzymes for use in allulose production pathways are provided in Table 1 below.

TABLE 1
Exemplary Allulose Pathway Enzymes

| | Enzyme | Enzyme | Gene Bank | |
|---|---|---|---|---|
| Enzyme Used | Code | Name | or Uniprot | Native Organism |
| α-glucan | A1 | TzAgp | WP_010477083 | Thermococcus zilligii |
| phosphorylase | A2 | PfAgp | WP_011012682 | Pyrococcus furiosus |
| | A3 | TcGlgP | AAV68178 | Thermus caldophilus |
| | A4 | PtAgp | WP_013931760 | Pseudothermotoga thermarum |
| | A5 | Tm08495 | AHD18925 | Thermotoga maritima |
| | A6 | AaGlgP | O66932 | Aquifex aeolicus |
| Cellodextrin | C1 | | BAB71818 | Clostridium thermocellum |
| phosphorylase | C2 | | GAE90338 | Clostridium straminisolvens |
| (also referred to as | C3 | | WP_011943512 | Thermotoga RQ2 |
| cellulose phosphorylase) | C4 | | ADM28607 | Ignisphaera aggregans |
| | C5 | | WP_004082399 | Thermotoga maritima |
| | C6 | | WP_013314871 | Spirochaeta thermophila |
| | C7 | | WP_015907054 | Caldicellulosiruptor bescii |
| | C8 | | WP_012548338 | Dictyoglomus thermophilum |
| | C9 | | WP_013297089 | Thermoanaerobacterium |
| | | | | thermosaccharolyticum |
| | C10 | | ACJ76363 | Thermosipho africanus |
| | C11 | | WP_006313788 | Caldisalinibacter kiritimatiensis |
| | C12 | | WP_058486419 | Defluviitalea phaphyphila |
| | C13 | | WP_013429146 | Caldicellulosiruptor kronotskyensis |
| | C14 | | WP_015848606 | Thermococcus sibiricus |
| | C15 | | WP_013129904 | Thermosphaera aggregans |
| Phosphoglucomutase | M1 | Tk1621 | Q68BJ6 | Thermococcus kodakaraensis |
| | M2 | Pk02350 | A0A127B864 | Pyrococcus kukulkanii |
| | M3 | Af0458 | WP_010877965 | Archaeoglobus fulgidus |
| | M4 | CtPgm2 | WP_003517493 | Clostridium thermocellum |
| | M5 | TtPgm2 | WP_011172742 | Thermus thermophilus |
| | M6 | TiManB | WP_022798653 | Thermus islandicus |
| Phosphoglucoisomerase | I1 | PfPgi | P83194 | Pyrococcus furiosus |
| | I2 | Ap0768 | BAA79746 | Aeropyrum pernix |
| | I3 | Cl1150 | WP_015232769 | Caldisphaera lagunensis |
| | I4 | CtPgi | A3DBX9 | Clostridium thermocellum |
| | I5 | TtPgi | WP_041443619 | Thermus thermophilus |
| | I6 | MjPgi | WP_010871130 | Methanococcus jannaschii |
| Allulose 6-phosphate | E1 | BtAlsE | WP_029098887 | Brevibacillus thermoruber |
| epimerase | E2 | Thebr1340 | WP_003868217 | Thermoanaerobacter brockii |
| | E3 | CasuRpe2 | WP_011025758 | Caldanaerobacter subterraneus |
| | E4 | Defds2125 | WP_013008817 | Deferribacter desulfuricans |
| | E5 | Hg1285 | WP_008287078 | Hydrogenivirga sp. 128-5-R1-1 |
| | E6 | Tthe1731 | WP_013298194 | Thermoanerobacterium |
| | | | | thermosaccharolyticum |
| | E7 | Theru00510 | WP_025305325 | Thermocrinis ruber |
| | E8 | That0313 | WP_073071389 | Thermosipho atlanticus |
| | E9 | ThtaRpe | WP_068550718 | Thermosulfidibacter takaii |

| | |
|---|---|
| Allulose 6-phosphate | A mutant A6PP comprising the amino acid sequence |
| phosphatase | of any one of SEQ ID NOs: 2-24 |

It should be understood that any combination of enzymes may be selected from Table 1. For example, a α-glucan phosphorylase may be selected from any one of TzAgp, PfAgp, TcGlgP, PtAgp, Tm08495, and AaGlgP and combined with a phosphoglucomutase selected from any one of Tk1621, Pk02350, Af0458, CtPgm2, TtPgm2, and TiManB and combined with any phosphoglucoisomerase in Table 1, any allulose 6-phosphate epimerase in Table 1, and any allulose 6-phosphate phosphatase comprising the amino acid sequence of any one of SEQ TD NOs: 2-24. In other embodiments, at least one α-glucan phosphorylase, at least one phosphoglucomutase, at least one phosphoglucoisomerase, at least one allulose 6-phosphate epimerase, and at least one allulose 6-phosphate phosphatase are used and selected from the enzymes appearing in Table 1. In other embodiments, enzymes from Table 1 are used in a combination of steps from Table 1 (e.g., steps 1 and 2 are carried out where a α-glucan phosphorylase is used in combination with a phosphoglucomutase to convert a polysaccharide to glucose 6-phosphate). In some embodiments, 2 steps are employed to perform a transformation. In other embodiments, only 3, 4, or 5 steps selected from the group containing Step 1, Step 2, Step 3, Step 4, and Step 5 are used to perform the corresponding transformations. In some embodiments, enzymes from Table 1 are used only to carry out a single step from Table 1 (e.g., only step 2 is carried out were a phosphoglucomutase, such as Tk1621, is used to convert glucose 1-phosphate to glucose 6-phosphate). In other embodiments, only a single step selected from the group consisting of Step 1, Step 2, Step 3, Step 4, and Step 5 is carried out.

In some embodiments, a Allulose 6-phosphate epimerase (e.g., Brevibacillus thermoruber Allulose 6-phosphate epimerase) is combined with any one of the mutant A6PPs of the disclosure (e.g., a mutant A6PP comprising the amino acid sequence of any one of SEQ ID NOs: 2-24; or a mutant A6PP having at least one amino acid mutation at position 41, 59, 89, 124, 140, and/or 142 relative to the amino acid numbering of SEQ ID NO: 1) and a substrate (e.g., fructose 6-phosphate).

### Substrate Flexibility and Debranching Enzymes

For all pathways described herein, a multitude of polysaccharide substrates can be used. Non-limiting examples of polymeric glucose substrates include starch, glycogen, and maltodextrin. In some embodiments, the substrate is starch. In other embodiments, the substrate is glycogen. In still other embodiments, the substrate is maltodextrin. In some embodiments, a partially hydrolyzed version of a polymeric glucose substrate (e.g., starch, glycogen, or maltodextrin) is used. Starch, glycogen, and maltodextrin include a plurality of glucose monomers linked primarily by α(1-4) bonds and some α(1-6) bonds. Both starch and glycogen contain these α(1-6) branch points, although glycogen is substantially more branched than starch. For the α(1-4) polymers, α-glucan phosphorylases consume the polymers one glucose at a time releasing glucose 1-phosphate.

Long polymers of starch are often insoluble in aqueous solutions and in addition to precipitating out, can cause gelling and retrogradation of the solution. When starch is partially hydrolyzed to smaller chain length polymers, either through chemical (e.g., acid hydrolysis) or enzymatic (e.g., α-amylase) methods, the resulting products are maltodextrins. These hydrolyzed derivatives often solubilize and mix better than their parent molecules, and thus, in some embodiments, are used in the pathways provided herein.

For glycogen, starch, or hydrolyzed maltodextrins, α(1-6) branches will substantially reduce yields of any allulose production pathway, as the glucan phosphorylase chew the polymers down to the end of their branches, leaving a large central core of available glucose unconverted. For these substrates/pathways, debranching enzymes may be used to increase substrate availability to the α-glucan phosphorylase. There are two exemplary classes of debranching enzymes that can be used: isoamylases and pullulanases. Enzymatically, both classes perform the same function but differ in substrate specificity. While using the debranching enzyme increases yields, the timing of the use will depend on the process and substrates being used. In some embodiments, an α-glucan is pretreated with α-amylase and a debranching enzyme, and then the resulting debranched maltodextrin(s) is fed into a reactor with the other pathway enzymes. In other embodiments, the debranching occurs concurrent with the pathway and branched α-glucans are fed into the reaction containing all pathway enzymes as well as the debranching enzyme.

Some aspects of the present disclosure provide methods, compositions, and systems for producing allulose. These methods, in some embodiments, include culturing cells engineered to express at least one debranching enzyme, at least one α-glucan phosphorylase, at least one phosphoglucomutase, at least one phosphoglucoisomerase, at least one allulose 6-phosphate epimerase, at least one allulose 6-phosphate phosphatase (e.g., a mutant A6PP comprising the amino acid sequence of any one of SEQ ID NOs: 2-24), or a combination of at least two (e.g., at least three, or at least four) of the foregoing enzymes. These methods, in some embodiments, include culturing cells engineered to express at least one debranching enzyme, at least one cellodextrin phosphorylase, at least one phosphoglucomutase, at least one phosphoglucoisomerase, at least one allulose 6-phosphate epimerase, at least one allulose 6-phosphate phosphatase (e.g., a mutant A6PP comprising the amino acid sequence of any one of SEQ ID NOs: 2-24), or a combination of at least two (e.g., at least three, or at least four) of the foregoing enzymes.

In some embodiments, at least one of the enzymes used to convert a polysaccharide to allulose is a thermostable enzyme. In some embodiments, at least two (e.g., at least three or at least four) of the enzymes are thermostable enzymes. In some embodiments, all of the enzymes are thermostable enzymes. Thus, in some embodiments, the methods include culturing cells engineered to express at least one thermostable debranching enzyme, at least one thermostable α-glucan phosphorylase, at least one thermostable phosphoglucomutase, at least one thermostable phosphoglucoisomerase, at least one thermostable allulose 6-phosphate epimerase, at least one thermostable allulose 6-phosphate phosphatase (e.g., a mutant A6PP comprising the amino acid sequence of any one of SEQ ID NOs: 2-24), or a combination of at least two or more of the foregoing thermostable enzymes.

In some embodiments, at least one of the enzymes used to convert cellulose/cellodextrin to allulose is a thermostable enzyme. In some embodiments, at least two (e.g., at least three or at least four) of the enzymes are thermostable enzymes. In some embodiments, all of the enzymes are thermostable enzymes. Thus, in some embodiments, the methods include culturing cells engineered to express at least one thermostable debranching enzyme, at least one thermostable cellodextrin phosphorylase, at least one thermostable phosphoglucomutase, at least one thermostable phosphoglucoisomerase, at least one thermostable allulose 6-phosphate epimerase, at least one thermostable allulose 6-phosphate phosphatase (e.g., a mutant A6PP comprising the amino acid sequence of any one of SEQ ID NOs: 2-24), or a combination of at least two or more of the foregoing thermostable enzymes.

In some embodiments, the methods of producing allulose include lysing (e.g., thermal, osmotic, mechanical, chemical, or enzymatic lysis) the cultured cells to produce at least one (e.g., at least two, at least three, or at least four) cell lysate. It should be understood that multiple cell lysates (and thus multiple cell populations, e.g., from the same organism (e.g., bacteria) or from different organisms (e.g., bacteria, yeast, and/or plant cells)) may be used in an enzymatic reaction as provided herein. For example, one cell population may be engineered to express one or more enzymes(s) of the allulose production pathway, while another cell population (or several other cell populations) may be engineered to express another (at least one other) enzyme of the allulose production pathway. Thus, in some embodiments, the methods comprise culturing at least one population of cells engineered to express at least one debranching enzyme, culturing at least one population of cells engineered to express at least one α-glucan phosphorylase, culturing at least one cell population engineered to express at least one phosphoglucomutase, culturing at least one cell population engineered to express at least one phosphoglucoisomerase, culturing at least one cell population engineered to express at least one allulose 6-phosphate epimerase, and/or culturing at least one cell population engineered to express at least one allulose 6-phosphate phosphatase (e.g., a mutant A6PP comprising the amino acid sequence of any one of SEQ ID NOs: 2-24). Thus, in some embodiments, the methods comprise culturing at least one population of cells engineered to express at least one debranching enzyme, culturing at least one population of cells engineered to express at least one cellodextrin phosphorylase, culturing at least one cell population engineered to express at least one phosphoglucomutase, culturing at least one cell population engineered to express at least one phosphoglucoisomerase, culturing at least one cell population engineered to express at least one allulose 6-phosphate epimerase, and/or culturing at least one cell population engineered to express at least one allulose 6-phosphate phosphatase (e.g., a mutant A6PP comprising the amino acid sequence of any one of SEQ ID NOs: 2-24). Following lysis of the cells, the cell lysates are combined such that the enzymes are present in a single cell lysate/reaction mixture.

A cell lysate or reaction mixture described herein is a mixture of cellular components and heterologous components that may include an enzyme for allulose production (e.g., a mutant A6PP), cell nutrients and cofactors, nucleic acids, cellular proteins, allulose production substrates (e.g., fructose 6-phosphate or allulose 6-phosphate), and cofactors.

Also provided herein are cells and cell lysates used for the production of allulose. Thus, an engineered cell (e.g., bacterial cell, yeast cell, and/or plant cell) or cell lysate(s) of the present disclosure may include at least one (e.g., at least two, at least three, or at least four) enzyme selected from the group consisting of debranching enzymes, α-glucan phosphorylases, phosphoglucomutases, phosphoglucoisomerases, allulose 6-phosphate epimerases, and allulose 6-phosphate phosphatase (e.g., a mutant A6PP comprising the amino acid sequence of any one of SEQ ID NOs: 2-24). In some embodiments, an engineered cell (e.g., bacterial cell, yeast cell, and/or plant cell) or cell lysate(s) of the present disclosure includes at least one (e.g., at least two, at least three, or at least four) enzyme selected from the group consisting of thermostable debranching enzymes, thermostable α-glucan phosphorylases, thermostable phosphoglucomutases, thermostable phosphoglucoisomerases, thermostable allulose 6-phosphate epimerases, and thermostable allulose 6-phosphate phosphatase (e.g., a mutant A6PP comprising the amino acid sequence of any one of SEQ ID NOs: 2-24).

Also provided herein are cells and cell lysates used for the production of allulose. Thus, an engineered cell (e.g., bacterial cell, yeast cell, and/or plant cell) or cell lysate(s) of the present disclosure may include at least one (e.g., at least two, at least three, or at least four) enzyme selected from the group consisting of debranching enzymes, cellodextrin phosphorylases, phosphoglucomutases, phosphoglucoisomerases, allulose 6-phosphate epimerases, and allulose 6-phosphate phosphatase (e.g., a mutant A6PP comprising the amino acid sequence of any one of SEQ ID NOs: 2-24). In some embodiments, an engineered cell (e.g., bacterial cell, yeast cell, and/or plant cell) or cell lysate(s) of the present disclosure includes at least one (e.g., at least two, at least three, or at least four) enzyme selected from the group consisting of thermostable debranching enzymes, thermostable cellodextrin phosphorylases, thermostable phosphoglucomutases, thermostable phosphoglucoisomerases, thermostable allulose 6-phosphate epimerases, and thermostable allulose 6-phosphate phosphatase (e.g., a mutant A6PP comprising the amino acid sequence of any one of SEQ ID NOs: 2-24).

Non-limiting examples of debranching enzymes for use in allulose production pathways are provided in Table 2 below.

TABLE 2
Exemplary Debranching Enzymes

| Debranching | | GeneBank or | |
|---|---|---|---|
| Enzyme | Enzyme Name | Uniprot | Native Organism |
| Pullulanase | Fp1793 | AAD30387 | Fervidobacterium pennavorans |
| | TRQ5Pul | WP_012310857 | Thermotoga sp. RQ5 |
| | BfPull | BAB18516 | Bacillus flavocaldarius |
| Isoamylase | StTreX | BAB65940 | Sulfolobus tokodaii |
| | MhTreX | AAS00512 | Metallosphaera hakonensis |
| | StGlgX | WP_012873143 | Sphaerobacter thermophilus |

In some embodiments for producing allulose, a α-glucan phosphorylase may be selected from any one of TzAgp, PfAgp, TcGlgP, PtAgp, Tm08495, and AaGlgP and combined with a phosphoglucomutase selected from any one of Tk1621, Pk02350, Af0458, CtPgm2, TtPgm2, and TiManB and combined with any phosphoglucoisomerase in Table 1, any allulose 6-phosphate epimerase in Table 1, any allulose 6-phosphate phosphatase in Table 1, and further comprise any enzymes selected from Table 2, such as a pullulanase or isoamylase. In other embodiments, at least one debranching enzyme, at least one α-glucan phosphorylase, at least one phosphoglucomutase, at least one phosphoglucoisomerase, at least one allulose 6-phosphate epimerase, and at least one allulose 6-phosphate phosphatase are used and selected from the enzymes appearing in Table 1 and Table 2.

### Cell-Free Production

“Cell-free production” is the use of biological processes for the synthesis of a biomolecule or chemical compound without using living cells. Rather, the cells are lysed and unpurified (crude) portions, containing enzymes, are used for the production of a desired product. As a non-limiting example, cells are cultured, harvested, and lysed by high-pressure homogenization. The cell-free reaction may be conducted in a batch or fed-batch mode. In some instances, the biological reaction networks fill the working volume of the reactor and may be more dilute than the intracellular environment. Yet substantially all of the cellular catalysts are provided, including catalysts that are membrane associated. The inner membrane is fragmented during cell lysis, and the fragments of these membranes form functional membrane vesicles. Thus, complex biotransformations are effected by catalysis. See, e.g., Swartz, AIChE Journal, 2012, 58(1), 5-13, incorporated herein by reference. In some embodiments, a cell-free production system contains cells that not not lysed (e.g., comprises a population of lysed cells and unlysed cells).

Cell-free methods and systems of the present disclosure, in some embodiments, utilize cell lysates (e.g., crude or partially purified cell lysates), discussed in greater detail herein. Cell lysates may be prepared, for example, by mechanical means (e.g., shearing or crushing). In some embodiments, cell lysates are distinct from chemically-permeabilized cells. As discussed here, in some embodiments, during cell lysis (e.g., mechanical cell lysis), the inner cell membrane is fragmented such that inverted membrane vesicles are formed in the cells lysates. Cells that are lysed (e.g., at least 75%, 80%, 85%, 90%, or 95%) are no longer intact.

In some embodiments, permeabilized cells are used. Permeabilized cells are intact cells containing perforations (small holes). In some embodiments, cells may be permeabilized to release the cell content for use in a reaction as provided herein. In some embodiments, lysed cells are used (and not permeabilized cells).

In some embodiments, partially purified cell fractions are used. A partially purified cell fraction is a cell lysate from which one or more cellular components (e.g., cell membranes) have been partially or completely removed.

### Thermostable Enzymes

An enzyme is considered thermostable if the enzyme (a) retains a substantial portion of its activity after exposure to high temperatures that denature other native enzymes or (b) functions at a relatively high rate after exposure to a medium to high temperature where native enzymes function at low rates.

In some embodiments, a thermostable enzyme retains greater than 50% activity following exposure to relatively high temperature that would otherwise denature a similar (non-thermostable) native enzyme. In some embodiments, a thermostable enzyme retains 50-100% activity following exposure to relatively high temperature that would otherwise denature a similar (non-thermostable) native enzyme. For example, a thermostable enzyme may retain 50-90%, 50-85%, 50-80%, 50-75%, 50-70%, 50-65%, 50-60%, or 50-55% of its activity following exposure to relatively high temperature that would otherwise denature a similar (non-thermostable) native enzyme. In some embodiments, a thermostable enzyme retains 25%, 30%, 35%, 40%, 45%, 50%, 55%, 60%, 65%, 70%, 75%, 80%, 85%, 90%, 95%, 98%, 99%, or 100% of its activity following exposure to relatively high temperature that would otherwise denature a similar (non-thermostable) native enzyme.

In some embodiments, the activity of a thermostable enzyme after exposure medium to high temperature is greater than (e.g., 25%, 30%, 35%, 40%, 45%, 50%, 55%, 60%, 65%, 70%, 75%, 80%, 85%, 90%, 95%, 98%, 99%, or 100% greater than) the activity of a similar (non-thermostable) native enzyme.

Thermostable enzymes (e.g., phosphatases or phosphorylases) may remain active (able to catalyze a reaction), for example, at temperatures of 45° C. to 80° C., or higher. In some embodiments, thermostable enzymes remain active at a temperature of 45-80° C., 45-70° C., 45-60° C., 45-50° C., 50-80° C., 50-70° C., 50-60° C., 60-80° C., 60-70° C., or 70- 80° C. For example, thermostable enzymes may remain active at a temperature of 45° C., 46° C., 47° C., 48° C., 49° C., 50° C., 51° C., 52° C., 53° C., 54° C., 55° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., or 80° C. Thermostable enzymes may remain active at relatively high temperatures for 15 minutes to 48 hours, or longer, after exposure to relatively high temperatures. For example, thermostable enzymes may remain active at relatively high temperatures for 1, 2, 3, 4, 5, 6, 7, 8, 9, 10, 11, 12, 13, 14, 15, 16, 17, 18, 19, 20, 24, 36, 42, or 48 hours.

### Engineered Cells

Engineered cells of the present disclosure, in some embodiments, comprise at least one, or all, of the enzymatic activities required to convert a polysaccharide and/or starch and/or maltodextrin to allulose. “Engineered cells” are cells that comprise at least one engineered (e.g., recombinant or synthetic) nucleic acid, or are otherwise modified such that they are structurally and/or functionally distinct from their naturally-occurring counterparts. Thus, a cell that contains an engineered nucleic acid is considered an “engineered cell.”

Engineered cells of the present disclosure, in some embodiments, comprise an α-glucan phosphorylase (e.g., a thermostable α-glucan phosphorylase), a phosphoglucomutase (e.g., a thermostable phosphoglucomutase), and at least one enzyme (e.g., thermostable enzyme) selected from the group consisting of phosphoglucoisomerases, allulose 6-phosphate 3-epimerases, allulose 6-phosphate phosphatase (e.g., a mutant A6PP comprising the amino acid sequence of any one of SEQ ID NOs: 2-24), and optionally a debranching enzyme.

Engineered cells of the present disclosure, in some embodiments, comprise a cellodextrin phosphorylase (e.g., a thermostable cellodextrin phosphorylase), a phosphoglucomutase (e.g., a thermostable phosphoglucomutase), and at least one enzyme (e.g., thermostable enzyme) selected from the group consisting of phosphoglucoisomerases, allulose 6-phosphate 3-epimerases, allulose 6-phosphate phosphatase (e.g., a mutant A6PP comprising the amino acid sequence of any one of SEQ ID NOs: 2-24), and optionally a debranching enzyme.

Engineered cells, in some embodiments, express selectable markers. Selectable markers are typically used to select engineered cells that have taken up and express an engineered nucleic acid following transfection of the cell (or following other procedures used to introduce foreign nucleic acid into the cell). Thus, a nucleic acid encoding product may also encode a selectable marker. Examples of selectable markers include, without limitation, antibiotic resistance free markers, genes encoding proteins that increase or decrease either resistance or sensitivity to antibiotics (e.g., ampicillin resistance genes, kanamycin resistance genes, neomycin resistance genes, tetracycline resistance genes and chloramphenicol resistance genes) or other compounds. Other selectable markers may be used in accordance with the present disclosure.

An engineered cell “expresses” a product if the product, encoded by a nucleic acid (e.g., an engineered nucleic acid), is produced in the cell. It is known in the art that gene expression refers to the process by which genetic instructions in the form of a nucleic acid are used to synthesize a product, such as a protein (e.g., an enzyme).

Engineered cells may be prokaryotic cells or eukaryotic cells. In some embodiments, engineered cells are bacterial cells, yeast cells, insect cells, mammalian cells, or other types of cells.

Engineered bacterial cells useful in the present disclosure include, without limitation, engineered Escherichia spp., Streptomyces spp., Zymonas spp., Acetobacter spp., Citrobacter spp., Synechocystis spp., Rhizobium spp., Clostridium spp., Corynebacterium spp., Streptococcus spp., Xanthomonas spp., Lactobacillus spp., Lactococcus spp., Bacillus spp., Alcaligenes spp., Pseudomonas spp., Aeromonas spp., Azotobacter spp., Comamonas spp., Mycobacterium spp., Rhodococcus spp., Gluconobacter spp., Ralstonia spp., Acidithiobacillus spp., Microlunatus spp., Geobacter spp., Geobacillus spp., Arthrobacter spp., Flavobacterium spp., Serratia spp., Saccharopolyspora spp., Thermus spp., Stenotrophomonas spp., Chromobacterium spp., Sinorhizobium spp., Saccharopolyspora spp., Agrobacterium spp., Vibrio spp., and Pantoea spp.

Engineered yeast cells useful in the present disclosure include, without limitation, engineered Saccharomyces spp., Schizosaccharomyces, Hansenula, Candida, Kluyveromyces, Yarrowia and Pichia.

In some embodiments, engineered cells useful in the present disclosure are engineered Escherichia coli cells, Bacillus subtilis cells, Pseudomonas putida cells, Saccharomyces cerevisiae cells, and/or Lactobacillus brevis cells. In some embodiments, engineered cells useful in the present disclosure are engineered Escherichia coli cells.

### Engineered Nucleic Acids

A “nucleic acid” is at least two nucleotides covalently linked together, and in some instances, may contain phosphodiester bonds (e.g., a phosphodiester “backbone”). Nucleic acids (e.g., components, or portions, of nucleic acids) may be naturally occurring or engineered. “Naturally occurring” nucleic acids are present in a cell that exists in nature in the absence of human intervention. “Engineered nucleic acids” include recombinant nucleic acids and synthetic nucleic acids. A “recombinant nucleic acid” refers to a molecule that is constructed by joining nucleic acid molecules (e.g., from the same species or from different species) and, typically, can be replicated in a living cell. A “synthetic nucleic acid” refers to a molecule that is biologically synthesized, chemically synthesized, or by other means synthesized or amplified. A synthetic nucleic acid includes nucleic acids that are chemically modified or otherwise modified but can base pair with naturally-occurring nucleic acid molecules. Recombinant and synthetic nucleic acids also include those molecules that result from the replication of either of the foregoing. Engineered nucleic acids may contain portions of nucleic acids that are naturally occurring, but as a whole, engineered nucleic acids do not occur naturally and require human intervention. In some embodiments, a nucleic acid encoding a product of the present disclosure is a recombinant nucleic acid or a synthetic nucleic acid. In other embodiments, a nucleic acid encoding a product is naturally occurring.

An engineered nucleic acid encoding enzymes, as provided herein, may be operably linked to a “promoter,” which is a control region of a nucleic acid at which initiation and rate of transcription of the remainder of a nucleic acid are controlled. A promoter drives expression or drives transcription of the nucleic acid that it regulates.

A promoter may be one naturally associated with a gene or sequence, as may be obtained by isolating the 5′ non-coding sequences located upstream of the coding segment of a given gene or sequence. Such a promoter can be referred to as “endogenous.”

In some embodiments, a coding nucleic acid sequence may be positioned under the control of a recombinant or heterologous promoter, which refers to a promoter that is not normally associated with the encoded sequence in its natural environment. Such promoters may include promoters of other genes; promoters isolated from any other cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR).

A promoter is considered to be “operably linked” when it is in a correct functional location and orientation in relation to the nucleic acid it regulates to control (“drive”) transcriptional initiation and/or expression of that nucleic acid.

Engineered nucleic acids of the present disclosure may contain a constitutive promoter or an inducible promoter. A “constitutive promoter” refers to a promoter that is constantly active in a cell. An “inducible promoter” refers to a promoter that initiates or enhances transcriptional activity when in the presence of, influenced by, or contacted by an inducer or inducing agent, or activated in the absence of a factor that causes repression. Inducible promoters for use in accordance with the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters, steroid-regulated promoters, metal-regulated promoters, pathogenesis-regulated promoters, temperature/heat-inducible, phosphate-regulated (e.g., PhoA), and light-regulated promoters.

An inducer or inducing agent may be endogenous or a normally exogenous condition (e.g., light), compound (e.g., chemical or non-chemical compound) or protein that contacts an inducible promoter in such a way as to be active in regulating transcriptional activity from the inducible promoter. Thus, a “signal that regulates transcription” of a nucleic acid refers to an inducer signal that acts on an inducible promoter. A signal that regulates transcription may activate or inactivate transcription, depending on the regulatory system used. Activation of transcription may involve directly acting on a promoter to drive transcription or indirectly acting on a promoter by inactivation of a repressor that is preventing the promoter from driving transcription. Conversely, deactivation of transcription may involve directly acting on a promoter to prevent transcription or indirectly acting on a promoter by activating a repressor that then acts on the promoter.

Engineered nucleic acids may be introduced into host cells using any means known in the art, including, without limitation, transformation, transfection (e.g., chemical (e.g., calcium phosphate, cationic polymers, or liposomes) or non-chemical (e.g., electroporation, sonoporation, impalefection, optical transfection)), and transduction (e.g., viral transduction).

Enzymes or other proteins encoded by a naturally-occurring, intracellular nucleic acid may be referred to as “endogenous enzymes” or “endogenous proteins.”

### Protease Targeting

Engineered cells of the present disclosure may express (e.g., endogenously express) enzymes necessary for the health of the cells that may have a negative impact on the production of a sugar of interest (e.g., allulose). Such enzymes are referred to herein as “target enzymes.” For example, target enzymes expressed by engineered cells may compete for substrates or cofactors with an enzyme that increases the rate of precursor supplied to a sugar production pathway. As another example, target enzymes expressed by the engineered cells may compete for substrates or cofactors with an enzyme that is a key pathway entry enzyme of a sugar production pathway. As yet another example, target enzymes expressed by the engineered cells may compete for substrates or cofactors with an enzyme that supplies a substrate or cofactor of a sugar production pathway.

To negate, or reduce, this negative impact, target enzymes can be modified to include a site-specific protease-recognition sequence in their protein sequence such that the target enzyme may be “targeted” and cleaved for inactivation during sugar production (see, e.g., U.S. Publication No. 2012/0052547 A1, published on Mar. 1, 2012; and International Publication No. WO 2015/021058 A2, published Feb. 12, 2015, each of which is incorporated by reference herein).

Cleavage of a target enzyme containing a site-specific protease-recognition sequence results from contact with a cognate site-specific protease that is sequestered in the periplasm of cell (separate from the target enzyme) during the cell growth phase (e.g., as engineered cells are cultured) and is brought into contact with the target enzyme during the conversion phase (e.g., following cell lysis to produce a cell lysate). Thus, engineered cells of the present disclosure comprise, in some embodiments, (i) an engineered nucleic acid encoding a target enzyme that negatively impacts the rate of conversion and includes a site-specific protease-recognition sequence in the protein sequence of the target enzyme, and (ii) an engineered nucleic acid encoding a site-specific protease that cleaves the site-specific protease-recognition sequence of the target enzyme and includes a periplasmic-targeting sequence. This periplasmic-targeting sequence is responsible for sequestering the site-specific protease to the periplasmic space of the cell until the cell is lysed. Examples of periplasmic-targeting sequences are provided below.

Examples of proteases that may be used in accordance with the present disclosure include, without limitation, alanine carboxypeptidase, astacin, bacterial leucyl aminopeptidase, cancer procoagulant, cathepsin B, clostripain, cytosol alanyl aminopeptidase, elastase, endoproteinase Brg-C, enterokinase, gastricsin, gelatinase, Gly-X carboxypeptidase, glycyl endopeptidase, human rhinovirus 3C protease, hypodermin C, Iga-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2B, picornain 3C, proendopeptidase, prolyl aminopeptidase, proprotein convertase I, proprotein convertase II, russellysin, saccharopepsin, semenogelase, T-plasminogen activator, thrombin, tissue kallikrein, tobacco etch virus (TEV), togavirin, tryptophanyl aminopeptidase, U-plasminogen activator, V8, venombin B, venombin BB and Xaa-pro aminopeptidase.

### Periplasmic Targeting

Enzymes of an allulose production pathway may include at least one enzyme that has a negative impact on the health (e.g., viability) of a cell. To negate or reduce this negative impact, an enzyme can be modified to include a relocation sequence such that the enzyme is relocated to a cellular or extra-cellular compartment where it is not naturally located and where the enzyme does not negatively impact the health of the cell (see, e.g., Publication No. US-2011-0275116-A1, published on Nov. 10, 2011, incorporated by reference herein). For example, an enzyme of an allulose production pathway may be relocated to the periplasmic space of a cell.

Thus, in some embodiments, engineered cells of the present disclosure comprise at least one enzyme of an allulose production pathway that is linked to a periplasmic-targeting sequence. A “periplasmic-targeting sequence” is an amino acid sequence that targets to the periplasm of a cell the protein to which it is linked. A protein that is linked to a periplasmic-targeting sequence will be sequestered in the periplasm of the cell in which the protein is expressed.

Periplasmic-targeting sequences may be derived from the N-terminus of bacterial secretory protein, for example. The sequences vary in length from about 15 to about 70 amino acids. The primary amino acid sequences of periplasmic-targeting sequences vary, but generally have a common structure, including the following components: (i) the N-terminal part has a variable length and generally carries a net positive charge; (ii) following is a central hydrophobic core of about 6 to about 15 amino acids; and (iii) the final component includes four to six amino acids which define the cleavage site for signal peptidases.

Periplasmic-targeting sequences of the present disclosure, in some embodiments, may be derived from a protein that is secreted in a gram-negative bacterium. The secreted protein may be encoded by the bacterium, or by a bacteriophage that infects the bacterium. Examples of gram-negative bacterial sources of secreted proteins include, without limitation, members of the genera Escherichia, Pseudomonas, Klebsiella, Salmonella, Caulobacter, Methylomonas, Acetobacter, Achromobacter, Acinetobacter, Aeromonas, Agrobacterium, Alcaligenes, Azotobacter, Burkholderia, Citrobacter, Comamonas, Enterobacter, Erwinia, Rhizobium, Vibrio, and Xanthomonas.

### Cell Cultures and Cell Lysates

Typically, engineered cells are cultured. “Culturing” refers to the process by which cells are grown under controlled conditions, typically outside of their natural environment. For example, engineered cells, such as engineered bacterial cells, may be grown as a cell suspension in liquid nutrient broth, also referred to as liquid “culture medium.” In some embodiments, unconverted starch is used as a substrate feed for growing cells.

Examples of commonly used bacterial Escherichia coli growth media include, without limitation, LB (Luria Bertani) Miller broth (1% NaCl): 1% peptone, 0.5% yeast extract, and 1% NaCl; LB (Luria Bertani) Lennox Broth (0.5% NaCl): 1% peptone, 0.5% yeast extract, and 0.5% NaCl; SOB medium (Super Optimal Broth): 2% peptone, 0.5% Yeast extract, 10 mM NaCl, 2.5 mM KCl, 10 mM MgCl2, 10 mM MgSO4; SOC medium (Super Optimal broth with Catabolic repressor): SOB+20 mM glucose; 2×YT broth (2× Yeast extract and Tryptone): 1.6% peptone, 1% yeast extract, and 0.5% NaCl; TB (Terrific Broth) medium: 1.2% peptone, 2.4% yeast extract, 72 mM K2HPO4, 17 mM KH2PO4 and 0.4% glycerol; and SB (Super Broth) medium: 3.2% peptone, 2% yeast extract, and 0.5% NaCl and or Korz medium (Korz, D J et al. 1995).

Examples of high density bacterial Escherichia coli growth media include, but are not limited to, DNAGro™ medium, ProGro™ medium, AutoX™ medium, DetoX™ medium, InduX™ medium, and SecPro™ medium.

In some embodiments, engineered cells are cultured under conditions that result in expression of enzymes or nucleic acids. Such culture conditions may depend on the particular product being expressed and the desired amount of the product.

In some embodiments, engineered cells are cultured at a temperature of 30° C. to 40° C. For example, engineered cells may be cultured at a temperature of 30° C., 31° C., 32° C., 33° C., 34° C., 35° C., 36° C., 37° C., 38° C., 39° C., or 40° C. Typically, engineered cells, such as engineered bacterial cells, are cultured at a temperature of 37° C.

In some embodiments, engineered cells are cultured for a period of time of 12 hours to 72 hours, or more. For example, engineered cells may be cultured for a period of time of 12, 18, 24, 30, 36, 42, 48, 54, 60, 66, or 72 hours. Typically, engineered cells, such as engineered bacterial cells, are cultured for a period of time of 12 to 24 hours. In some embodiments, engineered cells are cultured for 12 to 24 hours at a temperature of 37° C.

In some embodiments, engineered cells are cultured (e.g., in liquid cell culture medium) to an optical density, measured at a wavelength of 600 nm (OD600), of 5 to 25. In some embodiments, engineered cells are cultured to an OD600 of 5, 10, 15, 20, or 25.

In some embodiments, engineered cells are cultured to a density of 1×10⁴ to 1×10⁸ viable cells/ml cell culture medium. In some embodiments, engineered cells are cultured to a density of 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 1×10⁹, or 1×10¹⁰ viable cells/ml. In some embodiments, engineered cells are cultured to a density of 1×10⁸ to 1×10¹⁰ viable cells/ml. In some embodiments, engineered cells are cultured to a density of 2×10⁵ to 3×10⁷ viable cells/ml.

In some embodiments, engineered cells are cultured in a bioreactor. A bioreactor refers simply to a container in which cells are cultured, such as a culture flask, a dish, or a bag that may be single-use (disposable), autoclavable, or sterilizable. The bioreactor may be made of glass, or it may be polymer-based, or it may be made of other materials.

Examples of bioreactors include, without limitation, stirred tank (e.g., well mixed) bioreactors and tubular (e.g., plug flow) bioreactors, airlift bioreactors, membrane stirred tanks, spin filter stirred tanks, vibromixers, fluidized bed reactors, and membrane bioreactors. The mode of operating the bioreactor may be a batch or continuous processes and will depend on the engineered cells being cultured. A bioreactor is continuous when the feed and product streams are continuously being fed and withdrawn from the system. A batch bioreactor may have a continuous recirculating flow, but no continuous feeding of nutrient or product harvest. For intermittent-harvest and fed-batch (or batch fed) cultures, cells are inoculated at a lower viable cell density in a medium that is similar in composition to a batch medium. Cells are allowed to grow exponentially with essentially no external manipulation until nutrients are somewhat depleted and cells are approaching stationary growth phase. At this point, for an intermittent harvest batch-fed process, a portion of the cells and product may be harvested, and the removed culture medium is replenished with fresh medium. This process may be repeated several times. For production of recombinant proteins, a fed-batch process may be used. While cells are growing exponentially, but nutrients are becoming depleted, concentrated feed medium (e.g., 10-15 times concentrated basal medium) is added either continuously or intermittently to supply additional nutrients, allowing for further increase in cell concentration and the length of the conversion phase. Fresh medium may be added proportionally to cell concentration without removal of culture medium (broth). To accommodate the addition of medium, a fed-batch culture is started in a volume much lower that the full capacity of the bioreactor (e.g., approximately 40% to 50% of the maximum volume).

Some methods of the present disclosure are directed to large-scale production of sugar. For large-scale production methods, engineered cells may be grown in liquid culture medium in a volume of 5 liters (L) to 50 L, or more. In some embodiments, engineered cells may be grown in liquid culture medium in a volume of greater than (or equal to) 10 L. In some embodiments, engineered cells are grown in liquid culture medium in a volume of 5 L, 10 L, 15 L, 20 L, 25 L, 30 L, 35 L, 40 L, 45 L, 50 L, or more. In some embodiments, engineered cells may be grown in liquid culture medium in a volume of 5 L to 10 L, 5 L to 15 L, 5 L to 20 L, 5 L to 25 L, 5 L to 30 L, 5 L to 35 L, 5 L to 40 L, 5 L to 45 L, 10 L to 15 L, 10 L to 20 L, 10 L to 25 L, 20 L to 30 L, 10 L to 35 L, 10 L to 40 L, 10 L to 45 L, 10 L to 50 L, 15 L to 20 L, 15 L to 25 L, 15 L to 30 L, 15 L to 35 L, 15 L to 40 L, 15 L to 45 L, or 15 to 50 L.

Typically, culturing of engineered cells is followed by lysing the cells. “Lysing” refers to the process by which cells are broken down, for example, by viral, enzymatic, mechanical, chemical, heat or osmotic mechanisms. A “cell lysate” refers to a fluid containing the contents of lysed cells (e.g., lysed engineered cells), including, for example, organelles, membrane lipids, proteins, nucleic acids and inverted membrane vesicles. Cell lysates of the present disclosure may be produced by lysing any population of engineered cells, as provided herein. A “cell lysate” may exclude permeabilized/perforated cells.

Methods of cell lysis, referred to as “lysing,” are known in the art, any of which may be used in accordance with the present disclosure. Such cell lysis methods include, without limitation, physical/mechanical lysis, such as homogenization, as well as chemical, thermal, and/or enzymatic lysis.

Cell lysis can disturb carefully controlled cellular environments, resulting in protein degradation and modification by unregulated endogenous proteases and phosphatases. Thus, in some embodiments, protease inhibitors and/or phosphatase inhibitors may be added to the cell lysate or cells before lysis, or these activities may be removed by gene inactivation or protease targeting.

Cell lysates, in some embodiments, may be combined with at least one nutrient. For example, cell lysates may be combined with Na₂HPO₄, KH₂PO₄, NH₄Cl, NaCl, MgSO₄, and/or CaCl₂. Examples of other nutrients include, without limitation, magnesium sulfate, magnesium chloride, magnesium orotate, magnesium citrate, manganese chloride, calcium chloride, cobalt chloride, zinc chloride, zinc sulfate, potassium acetate, potassium phosphate monobasic, potassium phosphate dibasic, potassium phosphate tribasic, sodium acetate, sodium chloride, sodium phosphate monobasic, sodium phosphate dibasic, sodium phosphate tribasic, ammonium phosphate monobasic, ammonium phosphate dibasic, ammonium sulfate, ammonium chloride, ammonium hydroxide.

In some embodiments, cell lysates may consist of disrupted cell suspensions that are further modified by chemical, thermal, enzymatic or mechanical means to enrich or purify or reduce or eliminate specific components. For example, following disruption via mechanical, thermal, chemical or enzymatic means, as described above, the resulting material may be subjected to mechanical separation, e.g. membrane filtration, centrifugation or others, to partially enrich for a select enzymatic activity or to eliminate an undesired enzymatic activity or lysate component. Further examples may include the addition of salts or solvents to a disrupted cell suspension or alteration of the pH or temperature of the disrupted cell suspension resulting in the precipitation of desired activities followed by mechanical separation of these precipitated components as described above. Conversely, the addition of salts or solvents or the alteration of pH or temperature can be leveraged to eliminate undesired activities through either inactivation of those enzymes or precipitation and subsequent mechanical separation of the undesired enzymatic activity or activities.

Cell lysates, in some embodiments, may be combined with at least one cofactor. For example, cell lysates may be combined with adenosine diphosphate (ADP), adenosine triphosphate (ATP), nicotinamide adenine dinucleotide (NAD+), or other non-protein chemical compounds required for activity of an enzyme (e.g., inorganic ions and coenzymes).

In some embodiments, cell lysates are incubated under conditions that result in conversion of a polysaccharide or starch to sugar.

The volume of cell lysate used for a single reaction may vary. In some embodiments, the volume of a cell lysate is 1 to 150 m³. For example, the volume of a cell lysate may be 1 m³, 5 m³, 10 m³, 15 m³, 20 m³, 25 m³, 30 m³, 35 m³, 40 m³, 45 m³, 50 m³, 55 m³, 60 m³, 65 m³, 70 m³, 75 m³, 80 m³, 85 m³, 90 m³, 95 m³, 100 m³, 105 m³, 110 m³, 115 m³, 120 m³, 125 m³, 130 m³, 135 m³, 140 m³, 145 m³, or 150 m³. In some embodiments, the volume of a cell lysate is 25 m³ to 150 m³, 50 m³ to 150 m³, or 100 m³ to 150 m³.

### Purified Enzymes

In some embodiments of the present invention enzymes may be purified prior to addition to the production reaction. Enzyme purification should be understood to mean any enrichment or extraction of a specific enzyme or enzymatic activity or groups of enzymes or enzymatic activities from a complex mixture of materials, examples including, but not limited to, disrupted cell suspensions or cultured growth media. Thus, a purified enzyme or protein should be understood to be an enzyme or protein that has been separated or enriched from a complex matrix, whereby its relative concentration, as compared to other matrix components, is increased. Methods for purifying an enzyme include, but are not limited to, mechanical, chromatographic, chemical, pH or temperature means. For example, the addition of a salt to a disrupted cell suspension resulting in the precipitation of the target enzyme or protein followed by mechanical separation of the precipitated enzyme or protein, e.g., membrane filtration or centrifugation. Further examples may include the separation of an enzyme from a complex matrix through affinity based chromatographic methods (e.g. hexa-histidine-tag or streptavidin based purification).

### Enzymatic Specificity

Enzymatic specificity should be understood to be a trait inherent to an enzyme wherein it demonstrates improved reaction kinetics, thermodynamics or rates for one substrate as compared to another substrate. Enzymes with high specificity for a particular substrate are best exemplified by having a high ratio of catalytic rate (defined as turnover number or k_{cat}) to the Michaelis constant (Kₘ) or k_{cat}/Kₘ as compared to other substrates. It is advantageous to have an enzyme with high substrate specificity as this improves the rate of a reaction and improves yield by decreasing the production of non-target products. For example, the pathway described herein for the production of allulose has several intermediates that are similar in chemical structure, namely glucose 1-phosphate, glucose 6-phosphate, fructose 6-phosphate and allulose 6-phosphate. The ultimate enzymatic step in this process is the dephosphorylation of allulose 6-phosphate to the product allulose via an allulose 6-phsophate phosphatase. It is advantageous to utilize an enzyme with a very high-specificity for allulose 6-phosphate and a relatively low specificity for the other pathway intermediates, namely glucose 1-phosphate, glucose 6-phosphate and fructose 6-phosphate. Catalytic dephosphorylation of these intermediates would result in the production of either glucose or fructose thus decreasing yield and increasing product complexity.

### Kits

The kits described herein may include one or more containers housing components for performing the methods described herein and optionally instructions of uses. Any of the kit described herein may further comprise components needed for performing the methods. Each component of the kits, where applicable, may be provided in liquid form (e.g., in solution), or in solid form, (e.g., a dry powder). In certain cases, some of the components may be reconstitutable or otherwise processible (e.g., to an active form), for example, by the addition of a suitable solvent or other species (e.g., water or buffer), which may or may not be provided with the kit.

In some embodiments, the kits may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. As used herein, “promoted” includes all methods of doing business including methods of education, scientific inquiry, academic research, and any advertising or other promotional activity including written, oral and electronic communication of any form, associated with the disclosure. Additionally, the kits may include other components depending on the specific application, as described herein.

The kits may contain any one or more of the components described herein in one or more containers. The kits may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kits may also include other components, depending on the specific application, for example, containers, cell media, salts, buffers, reagents, etc.

### EXAMPLES

In order that the invention described herein may be more fully understood, the following examples are set forth. The synthetic and biological examples described in this application are offered to illustrate the methods provided herein and are not to be construed in any way as limiting their scope.

### Example 1. Production of Allulose 6-Phosphate Phosphatase (A6PP) Proteins

Allulose 6-phosphate phosphatase (A6PP) proteins having the amino acid sequences shown in Tables 3-6 were generated. The A6PP having the amino acid sequences of SEQ ID NOs: 1 is a wild-type Clostridium thermocellum A6PP. The A6PPs having any one of the amino acid sequences of SEQ ID: 2-24 are mutant A6PPs that are at least 85% identical to SEQ ID NO: 1. The A6PP having the amino acid sequences of SEQ ID NOs: 1 is a wild-type Clostridium thermocellum A6PP having an N-terminal hexahistidine purification tag. The A6PPs having any one of the amino acid sequences of SEQ ID: 23-44 are mutant A6PPs having an N-terminal hexahistidine purification tag that are at least 85% identical to SEQ ID NO: 25.

TABLE 3
Allulose 6-phosphate phosphatase (A6PP) proteins

| | A6PP substitutions | | |
|---|---|---|---|
| | relative to wild-type | | SEQ ID |
| | (SEQ ID NO: 1) | Amino Acid Sequence | NO: |
| Wild- | | MIKYKAVFFDFDYTLADSSKAVIECINYALQKMGYPESSPES | 1 |
| type | | ICRTIGLTLAEAFKILSGDTSDSNADLFRQYFKERADLVMCD | |
| | | RTVMYSTVECVLKKLKKADVKTGIVSTKYRYRIEDILKRDK | |
| | | LLQYFDVIVGGEDVAAHKPDPEGLLKAISMVGCQKEEVLFV | |
| | | GDSTVDARTAKNAGVDFVAVLTGTTGANEFSEYNPGAVIE | |
| | | DLSGLLDMFML | |
| | E41D, S59T, Y89F, | MIKYKAVFFDFDYTLADSSKAVIECINYALQKMGYPESSPDS | 2 |
| | D124H, A140T | ICRTIGLTLAEAFKILTGDTSDSNADLFRQYFKERADLVMCD | |
| | H142P | RTVMFSTVECVLKKLKKADVKTGIVSTKYRYRIEDILKRHK | |
| | | LLQYFDVIVGGEDVTAPKPDPEGLLKAISMVGCQKEEVLFV | |
| | | GDSTVDARTAKNAGVDFVAVLTGTTGANEFSEYNPGAVIE | |
| | | DLSGLLDMFML | |
| 413-0 | E41D, S59T, Y89F, | MIKYKAVFFDFDYTLADSSKAVIECINYALQKMGYPESSPDS | 3 |
| | D124H, A140T | ICRTIGLTLAEAYKILTGDTSDSNADLFAQYFKERADLVMCD | |
| | H142P, F55Y, R71A | RTVMFSTVECVLKKLKKADVKTGIVSTKYRYRIEDILKRHK | |
| | | LLQYFDVIVGGEDVTAPKPDPEGLLKAISMVGCQKEEVLFV | |
| | | GDSTVDARTAKNAGVDFVAVLTGTTGANEFSEYNPGAVIE | |
| | | DLSGLLDMFML | |
| 413-1 | E41D, S59T, Y89F, | MIKYKAVFFDFDYTLADSSKAVIECINYALQKMGYPESSPDS | 4 |
| | D124H, A140T | ICRTIGLTLAEAYKILTGDTSDSNADLFAQYFKERADLVMCD | |
| | H142P, F55Y, R71A, | RTVMFSTVECVLKKLKKADVKTGIVSTKYRYRIEAILKRHK | |
| | D119A | LLQYFDVIVGGEDVTAPKPDPEGLLKAISMVGCQKEEVLFV | |
| | | GDSTVDARTAKNAGVDFVAVLTGTTGANEFSEYNPGAVIE | |
| | | DLSGLLDMFML | |
| 413-3 | E41D, S59T, Y89F, | MIKYKAVFFDFDYTLADSSKAVIECINYALQKMGYPESSPDS | 5 |
| | D124H, A140T | ICRTIGLTLAEAYKILTGDTSDSNADLFAQYFKERADLVMCD | |
| | H142P, V134I | RTVMFSTVECVLKKLKKADVKTGIVSTKYRYRIEDILKRHK | |
| | | LLQYFDVIIGGEDVTAPKPDPEGLLKAISMVGCQKEEVLFVG | |
| | | DSTVDARTAKNAGVDFVAVLTGTTGANEFSEYNPGAVIEDL | |
| | | SGLLDMFML | |
| 413-4 | E41D, S59T, Y89F, | MIKYKAVFFDFDYTLADSSKAVIECINYALQKMGYPESSPDS | 6 |
| | D124H, A140T | ICRTIGLTLAEAYKILTGDTSDSNADLFAQYFKERADLVMCD | |
| | H142P, E137D | RTVMFSTVECVLKKLKKADVKTGIVSTKYRYRIEDILKRHK | |
| | | LLQYFDVIVGGDDVTAPKPDPEGLLKAISMVGCQKEEVLFV | |
| | | GDSTVDARTAKNAGVDFVAVLTGTTGANEFSEYNPGAVIE | |
| | | DLSGLLDMFML | |
| 413-5 | E41D, S59T, Y89F, | MIKYKAVFFDFDYTLADSSKAVIECINYALQKMGYPESSPDS | 7 |
| | D124H, A140T | ICRTIGLTLAEAYKILTGDTSDSNADLFAQYFKERADLVMCD | |
| | H142P, K101Q, | RTVMFSTVECVLKKLKQADVKTGIVSTKYRYRIEAILKRHK | |
| | D119A | LLQYFDVIVGGEDVTAPKPDPEGLLKAISMVGCQKEEVLFV | |
| | | GDSTVDARTAKNAGVDFVAVLTGTTGANEFSEYNPGAVIE | |
| | | DLSGLLDMFML | |
| 413-6 | E41D, S59T, Y89F, | MIKYKAVFFDFDYTLADSSKAVIECINYALQKMGYPESSPDS | 8 |
| | D124H, A140T | ICRTIGLTLAEAYKILTGDTSDSNADLFAQYFKERADLVMCD | |
| | H142P, D119A, | RTVMFSTVECVLKKLKKADVKTGIVSTKYRYRIEAILKRHK | |
| | V134I | LLQYFDVIIGGEDVTAPKPDPEGLLKAISMVGCQKEEVLFVG | |
| | | DSTVDARTAKNAGVDFVAVLTGTTGANEFSEYNPGAVIEDL | |
| | | SGLLDMFML | |
| 413-7 | E41D, S59T, Y89F, | MIKYKAVFFDFDYTLADSSKAVIECINYALQKMGYPESSPDS | 9 |
| | D124H, A140T | ICRTIGLTLAEAYKILTGDTSDSNADLFAQYFKERADLVMCD | |
| | H142P, D119A, | RTVMFSTVECVLKKLKKADVKTGIVSTKYRYRIEAILKRHK | |
| | E137D | LLQYFDVIVGGDDVTAPKPDPEGLLKAISMVGCQKEEVLFV | |
| | | GDSTVDARTAKNAGVDFVAVLTGTTGANEFSEYNPGAVIE | |
| | | DLSGLLDMFML | |
| 413-8 | E41D, S59T, Y89F, | MIKYKAVFFDFDYTLADSSKAVIECINYALQKMGYPESSPDS | 10 |
| | D124H, A140T | ICRTIGLTLAEAYKILTGDTSDSNADLFAQYFKERADLVMCD | |
| | H142P, K101Q, | RTVMFSTVECVLKKLKQADVKTGIVSTKYRYRIEDILKRHK | |
| | V134I | LLQYFDVIIGGEDVTAPKPDPEGLLKAISMVGCQKEEVLFVG | |
| | | DSTVDARTAKNAGVDFVAVLTGTTGANEFSEYNPGAVIEDL | |
| | | SGLLDMFML | |
| 413-9 | E41D, S59T, Y89F, | MIKYKAVFFDFDYTLADSSKAVIECINYALQKMGYPESSPDS | 11 |
| | D124H, A140T | ICRTIGLTLAEAYKILTGDTSDSNADLFAQYFKERADLVMCD | |
| | H142P, K101Q, | RTVMFSTVECVLKKLKQADVKTGIVSTKYRYRIEDILKRHK | |
| | E137D | LLQYFDVIVGGDDVTAPKPDPEGLLKAISMVGCQKEEVLFV | |
| | | GDSTVDARTAKNAGVDFVAVLTGTTGANEFSEYNPGAVIE | |
| | | DLSGLLDMFML | |
| 413- | E41D, S59T, Y89F, | MIKYKAVFFDFDYTLADSSKAVIECINYALQKMGYPESSPDS | 12 |
| 10 | D124H, A140T | ICRTIGLTLAEAYKILTGDTSDSNADLFAQYFKERADLVMCD | |
| | H142P, V134I, | RTVMFSTVECVLKKLKKADVKTGIVSTKYRYRIEDILKRHK | |
| | E137D | LLQYFDVIIGGDDVTAPKPDPEGLLKAISMVGCQKEEVLFVG | |
| | | DSTVDARTAKNAGVDFVAVLTGTTGANEFSEYNPGAVIEDL | |
| | | SGLLDMFML | |
| 413- | E41D, S59T, Y89F, | MIKYKAVFFDFDYTLADSSKAVIECINYALQKMGYPESSPDS | 13 |
| 11 | D124H, A140T | ICRTIGLTLAEAYKILTGDTSDSNADLFAQYFKERADLVMCD | |
| | H142P, K101Q, | RTVMFSTVECVLKKLKQADVKTGIVSTKYRYRIEAILKRHK | |
| | D119A, V134I | LLQYFDVIIGGEDVTAPKPDPEGLLKAISMVGCQKEEVLFVG | |
| | | DSTVDARTAKNAGVDFVAVLTGTTGANEFSEYNPGAVIEDL | |
| | | SGLLDMFML | |
| 413- | E41D, S59T, Y89F, | MIKYKAVFFDFDYTLADSSKAVIECINYALQKMGYPESSPDS | 14 |
| 12 | D124H, A140T | ICRTIGLTLAEAYKILTGDTSDSNADLFAQYFKERADLVMCD | |
| | H142P, K101Q, | RTVMFSTVECVLKKLKQADVKTGIVSTKYRYRIEAILKRHK | |
| | D119A, E137D | LLQYFDVIVGGDDVTAPKPDPEGLLKAISMVGCQKEEVLFV | |
| | | GDSTVDARTAKNAGVDFVAVLTGTTGANEFSEYNPGAVIE | |
| | | DLSGLLDMFML | |
| 413- | E41D, S59T, Y89F, | MIKYKAVFFDFDYTLADSSKAVIECINYALQKMGYPESSPDS | 15 |
| 13 | D124H, A140T | ICRTIGLTLAEAYKILTGDTSDSNADLFAQYFKERADLVMCD | |
| | H142P, D119A, | RTVMFSTVECVLKKLKKADVKTGIVSTKYRYRIEAILKRHK | |
| | V134I, E137D | LLQYFDVIIGGDDVTAPKPDPEGLLKAISMVGCQKEEVLFVG | |
| | | DSTVDARTAKNAGVDFVAVLTGTTGANEFSEYNPGAVIEDL | |
| | | SGLLDMFML | |
| 413- | E41D, S59T, Y89F, | MIKYKAVFFDFDYTLADSSKAVIECINYALQKMGYPESSPDS | 16 |
| 14 | D124H, A140T | ICRTIGLTLAEAYKILTGDTSDSNADLFAQYFKERADLVMCD | |
| | H142P, K101Q, | RTVMFSTVECVLKKLKQADVKTGIVSTKYRYRIEDILKRHK | |
| | V134I, E137D | LLQYFDVIIGGDDVTAPKPDPEGLLKAISMVGCQKEEVLFVG | |
| | | DSTVDARTAKNAGVDFVAVLTGTTGANEFSEYNPGAVIEDL | |
| | | SGLLDMFML | |
| 413- | E41D, S59T, Y89F, | MIKYKAVFFDFDYTLADSSKAVIECINYALQKMGYPESSPDS | 17 |
| 15 | D124H, A140T | ICRTIGLTLAEAYKILTGDTSDSNADLFAQYFKERADLVMCD | |
| | H142P, K101Q, | RTVMFSTVECVLKKLKQADVKTGIVSTKYRYRIEAILKRHK | |
| | D119A, V134I, | LLQYFDVIIGGDDVTAPKPDPEGLLKAISMVGCQKEEVLFVG | |
| | E137D | DSTVDARTAKNAGVDFVAVLTGTTGANEFSEYNPGAVIEDL | |
| | | SGLLDMFML | |
| 446-0 | E41D, F55Y, S59T, | MIKYKAVFFDFDYTLADSSKAVIECINYALQKMGYPESSPDS | 18 |
| | S65A, R71A, Y89F, | ICRTIGLTLAEAYKILTGDTSDANADLFAQYFKERADLVMC | |
| | K101Q, D124H, | DRTVMFSTVECVLKKLKQADVKTGIVSTKYRYRIEDILKRH | |
| | E137D, A140T, | KLLQYFDVIVGGDDVTAPKPDPEGLLKAISMVGCQKEEVLF | |
| | H142P | VGDSTVDARTAKNAGVDFVAVLTGTTGANEFSEYNPGAVI | |
| | | EDLSGLLDMFML | |
| 446-5 | E41D, F55Y, S59T, | MIKYKAVFFDFDYTLADSSKAVIECINYALQKMGYPESSPDS | 19 |
| | S65A, R71A, Y89F, | ICRTIGLTLAEAYKILTGDTSDANADLFAQYFKERADLVMC | |
| | K101Q, D124H, | DRTVMFSTVECVLKKLKQADVKTGIVSTKYRYRIEAILKRH | |
| | E137D, A140T, | KLLQYFDVIVGGDDVTAPKPDPEGLLKAISMVGCQKEEVLF | |
| | H142P, D119A | VGDSTVDARTAKNAGVDFVAVLTGTTGANEFSEYNPGAVI | |
| | | EDLSGLLDMFML | |
| 446-6 | E41D, F55Y, S59T, | MIKYKAVFFDFDYTLADSSKAVIECINYALQKMGYPESSPDS | 20 |
| | S65A, R71A, Y89F, | ICRTIGLTLAEAYKILTGDTSDANADLFAQYFKERADLVMC | |
| | K101Q, D124H, | DRTVMFSTVECVLKKLKQADVKTGIVSTKYRYRIEDILKRH | |
| | E137D, A140T, | KLLQYFDVIIGGDDVTAPKPDPEGLLKAISMVGCQKEEVLFV | |
| | H142P, V134I | GDSTVDARTAKNAGVDFVAVLTGTTGANEFSEYNPGAVIE | |
| | | DLSGLLDMFML | |
| 446-7 | E41D, F55Y, S59T, | MIKYKAVFFDFDYTLADSSKAVIECINYALQKMGYPESSPDS | 21 |
| | S65A, R71A, Y89F, | ICRTIGLTLAEAYKILTGDTSDANADLFAQYFKERADLVMC | |
| | K101Q, D124H, | DRTVMFSTVECVLKKLKQADVKTGIVSTKYRYRIEAILKRH | |
| | E137D, A140T, | KLLQYFDVIIGGDDVTAPKPDPEGLLKAISMVGCQKEEVLFV | |
| | H142P, D119A, | GDSTVDARTAKNAGVDFVAVLTGTTGANEFSEYNPGAVIE | |
| | V134I | DLSGLLDMFML | |
| 375 | S38C, E41D, T50S, | MIKYKAVFFDFDYTLADSSKAVIECINYALQKMGYPECSPD | 22 |
| | S59T, D119A, | SICRTIGLSLAEAFKILTGDTSDSNADLFRQYFKERADLVMC | |
| | D124H, A140T, | DRTVMYSTVECVLKKLKKADVKTGIVSTKYRYRIEAILKRH | |
| | H142P, E206P | KLLQYFDVIVGGEDVTAPKPDPEGLLKAISMVGCQKEEVLF | |
| | | VGDSTVDARTAKNAGVDFVAVLTGTTGANEFSEYNPGAVIP | |
| | | DLSGLLDMFML | |
| 377 | S38C, E41D, D119A, | MIKYKAVFFDFDYTLADSSKAVIECINYALQKMGYPECSPD | 23 |
| | D124H, A140T, | SICRTIGLTLAEAFKILSGDTSDSNADLFRQYFKERADLVMC | |
| | H142P, L150N, | DRTVMYSTVECVLKKLKKADVKTGIVSTKYRYRIEAILKRH | |
| | S197A, E206P | KLLQYFDVIVGGEDVTAPKPDPEGLNKAISMVGCQKEEVLF | |
| | | VGDSTVDARTAKNAGVDFVAVLTGTTGANEFAEYNPGAVI | |
| | | PDLSGLLDMFML | |
| 382 | S38C, E41D, S59T, | MIKYKAVFFDFDYTLADSSKAVIECINYALQKMGYPECSPD | 24 |
| | R71A, Y89F, | SICRTIGLTLAEAFKILTGDTSDSNADLFAQYFKERADLVMC | |
| | D124H, A140T, | DRTVMFSTVECVLKKLKKADVKTGIVSTKYRYRIEDILKRH | |
| | H142P, E206P | KLLQYFDVIVGGEDVTAPKPDPEGLLKAISMVGCQKEEVLF | |
| | | VGDSTVDARTAKNAGVDFVAVLTGTTGANEFSEYNPGAVIP | |
| | | DLSGLLDMFML | |

TABLE 4
Allulose 6-phosphate phosphatase (A6PP) proteins with hexahistidine purification tags

| | A6PP substitutions | | |
|---|---|---|---|
| | relative to wild-type | | SEQ ID |
| | (SEQ ID NO: 1) | Amino Acid Sequence | NO: |
| Wild- | | MHHHHHHIKYKAVFFDFDYTLADSSKAVIECINYALQKMG | 25 |
| type | | YPESSPESICRTIGLTLAEAFKILSGDTSDSNADLFRQYFKER | |
| | | ADLVMCDRTVMYSTVECVLKKLKKADVKTGIVSTKYRYRI | |
| | | EDILKRDKLLQYFDVIVGGEDVAAHKPDPEGLLKAISMVGC | |
| | | QKEEVLFVGDSTVDARTAKNAGVDFVAVLTGTTGANEFSE | |
| | | YNPGAVIEDLSGLLDMFML | |
| | E41D, S59T, Y89F, | MHHHHHHIKYKAVFFDFDYTLADSSKAVIECINYALQKMG | 26 |
| | D124H, A140T | YPESSPDSICRTIGLTLAEAFKILTGDTSDSNADLFRQYFKER | |
| | H142P | ADLVMCDRTVMFSTVECVLKKLKKADVKTGIVSTKYRYRI | |
| | | EDILKRHKLLQYFDVIVGGEDVTAPKPDPEGLLKAISMVGC | |
| | | QKEEVLFVGDSTVDARTAKNAGVDFVAVLTGTTGANEFSE | |
| | | YNPGAVIEDLSGLLDMFML | |
| 413-0 | E41D, S59T, Y89F, | MHHHHHHIKYKAVFFDFDYTLADSSKAVIECINYALQKMG | 27 |
| | D124H, A140T | YPESSPDSICRTIGLTLAEAYKILTGDTSDSNADLFAQYFKER | |
| | H142P, F55Y, R71A | ADLVMCDRTVMFSTVECVLKKLKKADVKTGIVSTKYRYRI | |
| | | EDILKRHKLLQYFDVIVGGEDVTAPKPDPEGLLKAISMVGC | |
| | | QKEEVLFVGDSTVDARTAKNAGVDFVAVLTGTTGANEFSE | |
| | | YNPGAVIEDLSGLLDMFML | |
| 413-1 | E41D, S59T, Y89F, | MHHHHHHIKYKAVFFDFDYTLADSSKAVIECINYALQKMG | 28 |
| | D124H, A140T | YPESSPDSICRTIGLTLAEAYKILTGDTSDSNADLFAQYFKER | |
| | H142P, F55Y, R71A, | ADLVMCDRTVMFSTVECVLKKLKKADVKTGIVSTKYRYRI | |
| | D119A | EAILKRHKLLQYFDVIVGGEDVTAPKPDPEGLLKAISMVGC | |
| | | QKEEVLFVGDSTVDARTAKNAGVDFVAVLTGTTGANEFSE | |
| | | YNPGAVIEDLSGLLDMFML | |
| 413-3 | E41D, S59T, Y89F, | MHHHHHHIKYKAVFFDFDYTLADSSKAVIECINYALQKMG | 29 |
| | D124H, A140T | YPESSPDSICRTIGLTLAEAYKILTGDTSDSNADLFAQYFKER | |
| | H142P, V134I | ADLVMCDRTVMFSTVECVLKKLKKADVKTGIVSTKYRYRI | |
| | | EDILKRHKLLQYFDVIIGGEDVTAPKPDPEGLLKAISMVGCQ | |
| | | KEEVLFVGDSTVDARTAKNAGVDFVAVLTGTTGANEFSEY | |
| | | NPGAVIEDLSGLLDMFML | |
| 413-4 | E41D, S59T, Y89F, | MHHHHHHIKYKAVFFDFDYTLADSSKAVIECINYALQKMG | 30 |
| | D124H, A140T | YPESSPDSICRTIGLTLAEAYKILTGDTSDSNADLFAQYFKER | |
| | H142P, E137D | ADLVMCDRTVMFSTVECVLKKLKKADVKTGIVSTKYRYRI | |
| | | EDILKRHKLLQYFDVIVGGDDVTAPKPDPEGLLKAISMVGC | |
| | | QKEEVLFVGDSTVDARTAKNAGVDFVAVLTGTTGANEFSE | |
| | | YNPGAVIEDLSGLLDMFML | |
| 413-5 | E41D, S59T, Y89F, | MHHHHHHIKYKAVFFDFDYTLADSSKAVIECINYALQKMG | 31 |
| | D124H, A140T | YPESSPDSICRTIGLTLAEAYKILTGDTSDSNADLFAQYFKER | |
| | H142P, K101Q, | ADLVMCDRTVMFSTVECVLKKLKQADVKTGIVSTKYRYRI | |
| | D119A | EAILKRHKLLQYFDVIVGGEDVTAPKPDPEGLLKAISMVGC | |
| | | QKEEVLFVGDSTVDARTAKNAGVDFVAVLTGTTGANEFSE | |
| | | YNPGAVIEDLSGLLDMFML | |
| 413-6 | E41D, S59T, Y89F, | MHHHHHHIKYKAVFFDFDYTLADSSKAVIECINYALQKMG | 32 |
| | D124H, A140T | YPESSPDSICRTIGLTLAEAYKILTGDTSDSNADLFAQYFKER | |
| | H142P, D119A, | ADLVMCDRTVMFSTVECVLKKLKKADVKTGIVSTKYRYRI | |
| | V134I | EAILKRHKLLQYFDVIIGGEDVTAPKPDPEGLLKAISMVGCQ | |
| | | KEEVLFVGDSTVDARTAKNAGVDFVAVLTGTTGANEFSEY | |
| | | NPGAVIEDLSGLLDMFML | |
| 413-7 | E41D, S59T, Y89F, | MHHHHHHIKYKAVFFDFDYTLADSSKAVIECINYALQKMG | 33 |
| | D124H, A140T | YPESSPDSICRTIGLTLAEAYKILTGDTSDSNADLFAQYFKER | |
| | H142P, D119A, | ADLVMCDRTVMFSTVECVLKKLKKADVKTGIVSTKYRYRI | |
| | E137D | EAILKRHKLLQYFDVIVGGDDVTAPKPDPEGLLKAISMVGC | |
| | | QKEEVLFVGDSTVDARTAKNAGVDFVAVLTGTTGANEFSE | |
| | | YNPGAVIEDLSGLLDMFML | |
| 413-8 | E41D, S59T, Y89F, | MHHHHHHIKYKAVFFDFDYTLADSSKAVIECINYALQKMG | 34 |
| | D124H, A140T | YPESSPDSICRTIGLTLAEAYKILTGDTSDSNADLFAQYFKER | |
| | H142P, K101Q, | ADLVMCDRTVMFSTVECVLKKLKQADVKTGIVSTKYRYRI | |
| | V134I | EDILKRHKLLQYFDVIIGGEDVTAPKPDPEGLLKAISMVGCQ | |
| | | KEEVLFVGDSTVDARTAKNAGVDFVAVLTGTTGANEFSEY | |
| | | NPGAVIEDLSGLLDMFML | |
| 413-9 | E41D, S59T, Y89F, | MHHHHHHIKYKAVFFDFDYTLADSSKAVIECINYALQKMG | 35 |
| | D124H, A140T | YPESSPDSICRTIGLTLAEAYKILTGDTSDSNADLFAQYFKER | |
| | H142P, K101Q, | ADLVMCDRTVMFSTVECVLKKLKQADVKTGIVSTKYRYRI | |
| | E137D | EDILKRHKLLQYFDVIVGGDDVTAPKPDPEGLLKAISMVGC | |
| | | QKEEVLFVGDSTVDARTAKNAGVDFVAVLTGTTGANEFSE | |
| | | YNPGAVIEDLSGLLDMFML | |
| 413- | E41D, S59T, Y89F, | MHHHHHHIKYKAVFFDFDYTLADSSKAVIECINYALQKMG | 36 |
| 10 | D124H, A140T | YPESSPDSICRTIGLTLAEAYKILTGDTSDSNADLFAQYFKER | |
| | H142P, V134I, | ADLVMCDRTVMFSTVECVLKKLKKADVKTGIVSTKYRYRI | |
| | E137D | EDILKRHKLLQYFDVIIGGDDVTAPKPDPEGLLKAISMVGCQ | |
| | | KEEVLFVGDSTVDARTAKNAGVDFVAVLTGTTGANEFSEY | |
| | | NPGAVIEDLSGLLDMFML | |
| 413- | E41D, S59T, Y89F, | MHHHHHHIKYKAVFFDFDYTLADSSKAVIECINYALQKMG | 37 |
| 11 | D124H, A140T | YPESSPDSICRTIGLTLAEAYKILTGDTSDSNADLFAQYFKER | |
| | H142P, K101Q, | ADLVMCDRTVMFSTVECVLKKLKQADVKTGIVSTKYRYRI | |
| | D119A, V134I | EAILKRHKLLQYFDVIIGGEDVTAPKPDPEGLLKAISMVGCQ | |
| | | KEEVLFVGDSTVDARTAKNAGVDFVAVLTGTTGANEFSEY | |
| | | NPGAVIEDLSGLLDMFML | |
| 413- | E41D, S59T, Y89F, | MHHHHHHIKYKAVFFDFDYTLADSSKAVIECINYALQKMG | 38 |
| 12 | D124H, A140T | YPESSPDSICRTIGLTLAEAYKILTGDTSDSNADLFAQYFKER | |
| | H142P, K101Q, | ADLVMCDRTVMFSTVECVLKKLKQADVKTGIVSTKYRYRI | |
| | D119A, E137D | EAILKRHKLLQYFDVIVGGDDVTAPKPDPEGLLKAISMVGC | |
| | | QKEEVLFVGDSTVDARTAKNAGVDFVAVLTGTTGANEFSE | |
| | | YNPGAVIEDLSGLLDMFML | |
| 413- | E41D, S59T, Y89F, | MHHHHHHIKYKAVFFDFDYTLADSSKAVIECINYALQKMG | 39 |
| 13 | D124H, A140T | YPESSPDSICRTIGLTLAEAYKILTGDTSDSNADLFAQYFKER | |
| | H142P, D119A, | ADLVMCDRTVMFSTVECVLKKLKKADVKTGIVSTKYRYRI | |
| | V134I, E137D | EAILKRHKLLQYFDVIIGGDDVTAPKPDPEGLLKAISMVGCQ | |
| | | KEEVLFVGDSTVDARTAKNAGVDFVAVLTGTTGANEFSEY | |
| | | NPGAVIEDLSGLLDMFML | |
| 413- | E41D, S59T, Y89F, | MHHHHHHIKYKAVFFDFDYTLADSSKAVIECINYALQKMG | 40 |
| 14 | D124H, A140T | YPESSPDSICRTIGLTLAEAYKILTGDTSDSNADLFAQYFKER | |
| | H142P, K101Q, | ADLVMCDRTVMFSTVECVLKKLKQADVKTGIVSTKYRYRI | |
| | V134I, E137D | EDILKRHKLLQYFDVIIGGDDVTAPKPDPEGLLKAISMVGCQ | |
| | | KEEVLFVGDSTVDARTAKNAGVDFVAVLTGTTGANEFSEY | |
| | | NPGAVIEDLSGLLDMFML | |
| 413- | E41D, S59T, Y89F, | MHHHHHHIKYKAVFFDFDYTLADSSKAVIECINYALQKMG | 41 |
| 15 | D124H, A140T | YPESSPDSICRTIGLTLAEAYKILTGDTSDSNADLFAQYFKER | |
| | H142P, K101Q, | ADLVMCDRTVMFSTVECVLKKLKQADVKTGIVSTKYRYRI | |
| | D119A, V134I, | EAILKRHKLLQYFDVIIGGDDVTAPKPDPEGLLKAISMVGCQ | |
| | E137D | KEEVLFVGDSTVDARTAKNAGVDFVAVLTGTTGANEFSEY | |
| | | NPGAVIEDLSGLLDMFML | |
| 446-0 | E41D, F55Y, S59T, | MHHHHHHIKYKAVFFDFDYTLADSSKAVIECINYALQKMG | 42 |
| | S65A, R71A, Y89F, | YPESSPDSICRTIGLTLAEAYKILTGDTSDANADLFAQYFKER | |
| | K101Q, D124H, | ADLVMCDRTVMFSTVECVLKKLKQADVKTGIVSTKYRYRI | |
| | E137D, A140T, | EDILKRHKLLQYFDVIVGGDDVTAPKPDPEGLLKAISMVGC | |
| | H142P | QKEEVLFVGDSTVDARTAKNAGVDFVAVLTGTTGANEFSE | |
| | | YNPGAVIEDLSGLLDMFML | |
| 446-5 | E41D, F55Y, S59T, | MHHHHHHIKYKAVFFDFDYTLADSSKAVIECINYALQKMG | 43 |
| | S65A, R71A, Y89F, | YPESSPDSICRTIGLTLAEAYKILTGDTSDANADLFAQYFKER | |
| | K101Q, D124H, | ADLVMCDRTVMFSTVECVLKKLKQADVKTGIVSTKYRYRI | |
| | E137D, A140T, | EAILKRHKLLQYFDVIVGGDDVTAPKPDPEGLLKAISMVGC | |
| | H142P, D119A | QKEEVLFVGDSTVDARTAKNAGVDFVAVLTGTTGANEFSE | |
| | | YNPGAVIEDLSGLLDMFML | |
| 446-6 | E41D, F55Y, S59T, | MHHHHHHIKYKAVFFDFDYTLADSSKAVIECINYALQKMG | 44 |
| | S65A, R71A, Y89F, | YPESSPDSICRTIGLTLAEAYKILTGDTSDANADLFAQYFKER | |
| | K101Q, D124H, | ADLVMCDRTVMFSTVECVLKKLKQADVKTGIVSTKYRYRI | |
| | E137D, A140T, | EDILKRHKLLQYFDVIIGGDDVTAPKPDPEGLLKAISMVGCQ | |
| | H142P, V134I | KEEVLFVGDSTVDARTAKNAGVDFVAVLTGTTGANEFSEY | |
| | | NPGAVIEDLSGLLDMFML | |
| 446-7 | E41D, F55Y, S59T, | MHHHHHHIKYKAVFFDFDYTLADSSKAVIECINYALQKMG | 45 |
| | S65A, R71A, Y89F, | YPESSPDSICRTIGLTLAEAYKILTGDTSDANADLFAQYFKER | |
| | K101Q, D124H, | ADLVMCDRTVMFSTVECVLKKLKQADVKTGIVSTKYRYRI | |
| | E137D, A140T, | EAILKRHKLLQYFDVIIGGDDVTAPKPDPEGLLKAISMVGCQ | |
| | H142P, D119A, | KEEVLFVGDSTVDARTAKNAGVDFVAVLTGTTGANEFSEY | |
| | V134I | NPGAVIEDLSGLLDMFML | |

TABLE 5
Allulose 6-phosphate phosphatases (A6PPs)-Nucleic acid sequences

| | A6PP amino acid | | |
|---|---|---|---|
| | substitutions relative | | |
| | to wild-type (SEQ ID | | SEQ ID |
| | NO: 1) | Nucleic Acid Sequence | NO: |
| Wild- | | ATGCATCACCATCACCACCACATCAAATACAAAGCCGTGTTTTT | 46 |
| type | | CGATTTCGATTATACCCTGGCAGATAGCAGCAAAGCAGTTATTG | |
| | | AATGTATTAACTACGCCCTGCAGAAAATGGGTTATCCGGAAAGC | |
| | | TCACCGGAAAGCATTTGTCGTACCATTGGTCTGACCCTGGCCGA | |
| | | AGCATTCAAAATTCTGAGCGGTGATACCAGCGATAGCAATGCAG | |
| | | ACCTGTTTCGTCAGTATTTCAAAGAACGTGCAGATCTGGTTATGT | |
| | | GTGATCGTACCGTTATGTATAGCACCGTTGAATGTGTTCTGAAA | |
| | | AAGCTGAAAAAAGCCGATGTGAAAACCGGTATTGTGAGCACCA | |
| | | AATATCGTTATCGCATTGAGGATATTCTGAAACGCGATAAACTG | |
| | | CTGCAGTATTTCGATGTTATTGTTGGTGGTGAAGATGTGGCAGC | |
| | | ACATAAACCGGATCCTGAAGGTCTGCTGAAAGCAATTAGCATGG | |
| | | TTGGTTGTCAGAAAGAAGAGGTTCTGTTTGTTGGTGATTCAACC | |
| | | GTTGATGCACGTACCGCAAAAAATGCCGGTGTTGATTTTGTTGC | |
| | | AGTTCTGACCGGCACCACCGGTGCAAATGAATTTTCTGAATATA | |
| | | ATCCGGGTGCCGTGATTGAAGATCTGAGTGGTCTGCTGGATATG | |
| | | TTTATGCTGTAA | |
| | E41D, S59T, Y89F, | ATGATCAAGTATAAAGCCGTGTTTTTCGACTTCGACTATACGTTG | 47 |
| | D124H, A140T | GCCGATAGCAGCAAAGCTGTTATTGAGTGCATCAATTATGCGCT | |
| | H142P | GCAGAAAATGGGTTACCCGGAGAGCAGCCCGGAGTCTATTTGTC | |
| | | GTACCATTGGCCTGACGTTGGCAGAGGCATTCAAAATCCTGAGC | |
| | | GGCGATACCAGCGACAGCAACGCAGATCTGTTTCGTCAATACTT | |
| | | TAAGGAACGTGCGGATCTGGTTATGTGTGACCGCACCGTTATGT | |
| | | ACAGCACCGTTGAGTGCGTCCTGAAGAAACTGAAAAAAGCCGA | |
| | | TGTTAAGACCGGTATTGTCTCCACGAAATACCGCTACCGCATCG | |
| | | AAGATATCTTAAAGCGTGACAAGCTGCTCCAGTATTTTGACGTC | |
| | | ATCGTGGGTGGCGAAGATGTGGCGGCGCACAAGCCAGACCCGG | |
| | | AAGGCCTGCTGAAGGCAATTAGCATGGTCGGTTGCCAAAAGGA | |
| | | AGAGGTTCTGTTCGTTGGTGACAGCACTGTGGACGCGCGTACGG | |
| | | CGAAAAACGCTGGCGTGGACTTTGTGGCAGTCTTGACCGGCACC | |
| | | ACCGGTGCGAATGAATTCTCGGAGTACAACCCGGGTGCGGTAAT | |
| | | TGAAGATCTGTCCGGTCTGCTGGATATGTTCATGCTGTGATAA | |
| 413-0 | E41D, S59T, Y89F, | ATGATCAAGTATAAAGCCGTGTTTTTCGACTTCGACTATACGTTG | 48 |
| | D124H, A140T | GCCGATAGCAGCAAAGCTGTTATTGAGTGCATCAATTATGCGCT | |
| | H142P, F55Y, R71A | GCAGAAAATGGGTTACCCGGAGAGCAGCCCGGACTCTATTTGTC | |
| | | GTACCATTGGCCTGACGTTGGCAGAGGCATACAAAATCCTGACC | |
| | | GGCGATACCAGCGACAGCAACGCAGATCTGTTTGCGCAATACTT | |
| | | TAAGGAACGTGCGGATCTGGTTATGTGTGACCGCACCGTTATGT | |
| | | TCAGCACCGTTGAGTGCGTCCTGAAGAAACTGAAAAAAGCCGAT | |
| | | GTTAAGACCGGTATTGTCTCCACGAAATACCGCTACCGCATCGA | |
| | | AGATATCTTAAAGCGTCACAAGCTGCTCCAGTATTTTGACGTCA | |
| | | TCGTGGGTGGCGAAGATGTGACCGCGCCGAAGCCAGACCCGGA | |
| | | AGGCCTGCTGAAGGCAATTAGCATGGTCGGTTGCCAAAAGGAA | |
| | | GAGGTTCTGTTCGTTGGTGACAGCACTGTGGACGCGCGTACGGC | |
| | | GAAAAACGCTGGCGTGGACTTTGTGGCAGTCTTGACCGGCACCA | |
| | | CCGGTGCGAATGAATTCTCGGAGTACAACCCGGGTGCGGTAATT | |
| | | GAGGATCTGTCCGGTCTGCTGGATATGTTCATGCTGTGATAA | |
| 413-1 | E41D, S59T, Y89F, | ATGATCAAGTATAAAGCCGTGTTTTTCGACTTCGACTATACGTTG | 49 |
| | D124H, A140T | GCCGATAGCAGCAAAGCTGTTATTGAGTGCATCAATTATGCGCT | |
| | H142P, F55Y, R71A, | GCAGAAAATGGGTTACCCGGAGAGCAGCCCGGACTCTATTTGTC | |
| | D119A | GTACCATTGGCCTGACGTTGGCAGAGGCATACAAAATCCTGACC | |
| | | GGCGATACCAGCGACAGCAACGCAGATCTGTTTGCGCAATACTT | |
| | | TAAGGAACGTGCGGATCTGGTTATGTGTGACCGCACCGTTATGT | |
| | | TCAGCACCGTTGAGTGCGTCCTGAAGAAACTGAAAAAAGCCGAT | |
| | | GTTAAGACCGGTATTGTCTCCACGAAATACCGCTACCGCATCGA | |
| | | AGCGATCTTAAAGCGTCACAAGCTGCTCCAGTATTTTGACGTCA | |
| | | TCGTGGGTGGCGAAGATGTGACCGCGCCGAAGCCAGACCCGGA | |
| | | AGGCCTGCTGAAGGCAATTAGCATGGTCGGTTGCCAAAAGGAA | |
| | | GAGGTTCTGTTCGTTGGTGACAGCACTGTGGACGCGCGTACGGC | |
| | | GAAAAACGCTGGCGTGGACTTTGTGGCAGTCTTGACCGGCACCA | |
| | | CCGGTGCGAATGAATTCTCGGAGTACAACCCGGGTGCGGTAATT | |
| | | GAAGATCTGTCCGGTCTGCTGGATATGTTCATGCTGTGATAA | |
| 413-3 | E41D, S59T, Y89F, | ATGATCAAGTATAAAGCCGTGTTTTTCGACTTCGACTATACGTTG | 50 |
| | D124H, A140T | GCCGATAGCAGCAAAGCTGTTATTGAGTGCATCAATTATGCGCT | |
| | H142P, V134I | GCAGAAAATGGGTTACCCGGAGAGCAGCCCGGACTCTATTTGTC | |
| | | GTACCATTGGCCTGACGTTGGCAGAGGCATACAAAATCCTGACC | |
| | | GGCGATACCAGCGACAGCAACGCAGATCTGTTTGCGCAATACTT | |
| | | TAAGGAACGTGCGGATCTGGTTATGTGTGACCGCACCGTTATGT | |
| | | TCAGCACCGTTGAGTGCGTCCTGAAGAAACTGAAAAAAGCCGAT | |
| | | GTTAAGACCGGTATTGTCTCCACGAAATACCGCTACCGCATCGA | |
| | | AGATATCTTAAAGCGTCACAAGCTGCTCCAGTATTTTGACGTCA | |
| | | TCATCGGTGGCGAAGATGTGACCGCGCCGAAGCCAGACCCGGA | |
| | | AGGCCTGCTGAAGGCAATTAGCATGGTCGGTTGCCAAAAGGAA | |
| | | GAGGTTCTGTTCGTTGGTGACAGCACTGTGGACGCGCGTACGGC | |
| | | GAAAAACGCTGGCGTGGACTTTGTGGCAGTCTTGACCGGCACCA | |
| | | CCGGTGCGAATGAATTCTCGGAGTACAACCCGGGTGCGGTAATT | |
| | | GAAGATCTGTCCGGTCTGCTGGATATGTTCATGCTGTGATAA | |
| 413-4 | E41D, S59T, Y89F, | ATGATCAAGTATAAAGCCGTGTTTTTCGACTTCGACTATACGTTG | 51 |
| | D124H, A140T | GCCGATAGCAGCAAAGCTGTTATTGAGTGCATCAATTATGCGCT | |
| | H142P, E137D | GCAGAAAATGGGTTACCCGGAGAGCAGCCCGGACTCTATTTGTC | |
| | | GTACCATTGGCCTGACGTTGGCAGAGGCATACAAAATCCTGACC | |
| | | GGCGATACCAGCGACAGCAACGCAGATCTGTTTGCGCAATACTT | |
| | | TAAGGAACGTGCGGATCTGGTTATGTGTGACCGCACCGTTATGT | |
| | | TCAGCACCGTTGAGTGCGTCCTGAAGAAACTGAAAAAAGCCGAT | |
| | | GTTAAGACCGGTATTGTCTCCACGAAATACCGCTACCGCATCGA | |
| | | AGATATCTTAAAGCGTCACAAGCTGCTCCAGTATTTTGACGTCA | |
| | | TCGTGGGTGGCGACGATGTGACCGCGCCGAAGCCAGACCCGGA | |
| | | AGGCCTGCTGAAGGCAATTAGCATGGTCGGTTGCCAAAAGGAA | |
| | | GAGGTTCTGTTCGTTGGTGACAGCACTGTGGACGCGCGTACGGC | |
| | | GAAAAACGCTGGCGTGGACTTTGTGGCAGTCTTGACCGGCACCA | |
| | | CCGGTGCGAATGAATTCTCGGAGTACAACCCGGGTGCGGTAATT | |
| | | GAAGATCTGTCCGGTCTGCTGGATATGTTCATGCTGTGATAA | |
| 413-5 | E41D, S59T, Y89F, | ATGATCAAGTATAAAGCCGTGTTTTTCGACTTCGACTATACGTTG | 52 |
| | D124H, A140T | GCCGATAGCAGCAAAGCTGTTATTGAGTGCATCAATTATGCGCT | |
| | H142P, K101Q, | GCAGAAAATGGGTTACCCGGAGAGCAGCCCGGACTCTATTTGTC | |
| | D119A | GTACCATTGGCCTGACGTTGGCAGAGGCATACAAAATCCTGACC | |
| | | GGCGATACCAGCGACAGCAACGCAGATCTGTTTGCGCAATACTT | |
| | | TAAGGAACGTGCGGATCTGGTTATGTGTGACCGCACCGTTATGT | |
| | | TCAGCACCGTTGAGTGCGTCCTGAAGAAACTGAAACAGGCCGAT | |
| | | GTTAAGACCGGTATTGTCTCCACGAAATACCGCTACCGCATCGA | |
| | | AGCGATCTTAAAGCGTCACAAGCTGCTCCAGTATTTTGACGTCA | |
| | | TCGTGGGTGGCGAAGATGTGACCGCGCCGAAGCCAGACCCGGA | |
| | | AGGCCTGCTGAAGGCAATTAGCATGGTCGGTTGCCAAAAGGAA | |
| | | GAGGTTCTGTTCGTTGGTGACAGCACTGTGGACGCGCGTACGGC | |
| | | GAAAAACGCTGGCGTGGACTTTGTGGCAGTCTTGACCGGCACCA | |
| | | CCGGTGCGAATGAATTCTCGGAGTACAACCCGGGTGCGGTAATT | |
| | | GAAGATCTGTCCGGTCTGCTGGATATGTTCATGCTGTGATAA | |
| 413-6 | E41D, S59T, Y89F, | ATGATCAAGTATAAAGCCGTGTTTTTCGACTTCGACTATACGTTG | 53 |
| | D124H, A140T | GCCGATAGCAGCAAAGCTGTTATTGAGTGCATCAATTATGCGCT | |
| | H142P, D119A, | GCAGAAAATGGGTTACCCGGAGAGCAGCCCGGACTCTATTTGTC | |
| | V134I | GTACCATTGGCCTGACGTTGGCAGAGGCATACAAAATCCTGACC | |
| | | GGCGATACCAGCGACAGCAACGCAGATCTGTTTGCGCAATACTT | |
| | | TAAGGAACGTGCGGATCTGGTTATGTGTGACCGCACCGTTATGT | |
| | | TCAGCACCGTTGAGTGCGTCCTGAAGAAACTGAAAAAAGCCGAT | |
| | | GTTAAGACCGGTATTGTCTCCACGAAATACCGCTACCGCATCGA | |
| | | AGCGATCTTAAAGCGTCACAAGCTGCTCCAGTATTTTGACGTCA | |
| | | TCATCGGTGGCGAAGATGTGACCGCGCCGAAGCCAGACCCGGA | |
| | | AGGCCTGCTGAAGGCAATTAGCATGGTCGGTTGCCAAAAGGAA | |
| | | GAGGTTCTGTTCGTTGGTGACAGCACTGTGGACGCGCGTACGGC | |
| | | GAAAAACGCTGGCGTGGACTTTGTGGCAGTCTTGACCGGCACCA | |
| | | CCGGTGCGAATGAATTCTCGGAGTACAACCCGGGTGCGGTAATT | |
| | | GAAGATCTGTCCGGTCTGCTGGATATGTTCATGCTGTGATAA | |
| 413-7 | E41D, S59T, Y89F, | ATGATCAAGTATAAAGCCGTGTTTTTCGACTTCGACTATACGTTG | 54 |
| | D124H, A140T | GCCGATAGCAGCAAAGCTGTTATTGAGTGCATCAATTATGCGCT | |
| | H142P, D119A, | GCAGAAAATGGGTTACCCGGAGAGCAGCCCGGACTCTATTTGTC | |
| | E137D | GTACCATTGGCCTGACGTTGGCAGAGGCATACAAAATCCTGACC | |
| | | GGCGATACCAGCGACAGCAACGCAGATCTGTTTGCGCAATACTT | |
| | | TAAGGAACGTGCGGATCTGGTTATGTGTGACCGCACCGTTATGT | |
| | | TCAGCACCGTTGAGTGCGTCCTGAAGAAACTGAAAAAAGCCGAT | |
| | | GTTAAGACCGGTATTGTCTCCACGAAATACCGCTACCGCATCGA | |
| | | AGCGATCTTAAAGCGTCACAAGCTGCTCCAGTATTTTGACGTCA | |
| | | TCGTGGGTGGCGACGATGTGACCGCGCCGAAGCCAGACCCGGA | |
| | | AGGCCTGCTGAAGGCAATTAGCATGGTCGGTTGCCAAAAGGAA | |
| | | GAGGTTCTGTTCGTTGGTGACAGCACTGTGGACGCGCGTACGGC | |
| | | GAAAAACGCTGGCGTGGACTTTGTGGCAGTCTTGACCGGCACCA | |
| | | CCGGTGCGAATGAATTCTCGGAGTACAACCCGGGTGCGGTAATT | |
| | | GAAGATCTGTCCGGTCTGCTGGATATGTTCATGCTGTGATAA | |
| 413-8 | E41D, S59T, Y89F, | ATGATCAAGTATAAAGCCGTGTTTTTCGACTTCGACTATACGTTG | 55 |
| | D124H, A140T | GCCGATAGCAGCAAAGCTGTTATTGAGTGCATCAATTATGCGCT | |
| | H142P, K101Q, | GCAGAAAATGGGTTACCCGGAGAGCAGCCCGGACTCTATTTGTC | |
| | V134I | GTACCATTGGCCTGACGTTGGCAGAGGCATACAAAATCCTGACC | |
| | | GGCGATACCAGCGACAGCAACGCAGATCTGTTTGCGCAATACTT | |
| | | TAAGGAACGTGCGGATCTGGTTATGTGTGACCGCACCGTTATGT | |
| | | TCAGCACCGTTGAGTGCGTCCTGAAGAAACTGAAACAGGCCGAT | |
| | | GTTAAGACCGGTATTGTCTCCACGAAATACCGCTACCGCATCGA | |
| | | AGATATCTTAAAGCGTCACAAGCTGCTCCAGTATTTTGACGTCA | |
| | | TCATCGGTGGCGAAGATGTGACCGCGCCGAAGCCAGACCCGGA | |
| | | AGGCCTGCTGAAGGCAATTAGCATGGTCGGTTGCCAAAAGGAA | |
| | | GAGGTTCTGTTCGTTGGTGACAGCACTGTGGACGCGCGTACGGC | |
| | | GAAAAACGCTGGCGTGGACTTTGTGGCAGTCTTGACCGGCACCA | |
| | | CCGGTGCGAATGAATTCTCGGAGTACAACCCGGGTGCGGTAATT | |
| | | GAAGATCTGTCCGGTCTGCTGGATATGTTCATGCTGTGATAA | |
| 413-9 | E41D, S59T, Y89F, | ATGATCAAGTATAAAGCCGTGTTTTTCGACTTCGACTATACGTTG | 56 |
| | D124H, A140T | GCCGATAGCAGCAAAGCTGTTATTGAGTGCATCAATTATGCGCT | |
| | H142P, K101Q, | GCAGAAAATGGGTTACCCGGAGAGCAGCCCGGACTCTATTTGTC | |
| | E137D | GTACCATTGGCCTGACGTTGGCAGAGGCATACAAAATCCTGACC | |
| | | GGCGATACCAGCGACAGCAACGCAGATCTGTTTGCGCAATACTT | |
| | | TAAGGAACGTGCGGATCTGGTTATGTGTGACCGCACCGTTATGT | |
| | | TCAGCACCGTTGAGTGCGTCCTGAAGAAACTGAAACAGGCCGAT | |
| | | GTTAAGACCGGTATTGTCTCCACGAAATACCGCTACCGCATCGA | |
| | | AGATATCTTAAAGCGTCACAAGCTGCTCCAGTATTTTGACGTCA | |
| | | TCGTGGGTGGCGACGATGTGACCGCGCCGAAGCCAGACCCGGA | |
| | | AGGCCTGCTGAAGGCAATTAGCATGGTCGGTTGCCAAAAGGAA | |
| | | GAGGTTCTGTTCGTTGGTGACAGCACTGTGGACGCGCGTACGGC | |
| | | GAAAAACGCTGGCGTGGACTTTGTGGCAGTCTTGACCGGCACCA | |
| | | CCGGTGCGAATGAATTCTCGGAGTACAACCCGGGTGCGGTAATT | |
| | | GAAGATCTGTCCGGTCTGCTGGATATGTTCATGCTGTGATAA | |
| 413- | E41D, S59T, Y89F, | ATGATCAAGTATAAAGCCGTGTTTTTCGACTTCGACTATACGTTG | 57 |
| 10 | D124H, A140T | GCCGATAGCAGCAAAGCTGTTATTGAGTGCATCAATTATGCGCT | |
| | H142P, V134I, | GCAGAAAATGGGTTACCCGGAGAGCAGCCCGGACTCTATTTGTC | |
| | E137D | GTACCATTGGCCTGACGTTGGCAGAGGCATACAAAATCCTGACC | |
| | | GGCGATACCAGCGACAGCAACGCAGATCTGTTTGCGCAATACTT | |
| | | TAAGGAACGTGCGGATCTGGTTATGTGTGACCGCACCGTTATGT | |
| | | TCAGCACCGTTGAGTGCGTCCTGAAGAAACTGAAAAAAGCCGAT | |
| | | GTTAAGACCGGTATTGTCTCCACGAAATACCGCTACCGCATCGA | |
| | | AGATATCTTAAAGCGTCACAAGCTGCTCCAGTATTTTGACGTCA | |
| | | TCATCGGTGGCGACGATGTGACCGCGCCGAAGCCAGACCCGGA | |
| | | AGGCCTGCTGAAGGCAATTAGCATGGTCGGTTGCCAAAAGGAA | |
| | | GAGGTTCTGTTCGTTGGTGACAGCACTGTGGACGCGCGTACGGC | |
| | | GAAAAACGCTGGCGTGGACTTTGTGGCAGTCTTGACCGGCACCA | |
| | | CCGGTGCGAATGAATTCTCGGAGTACAACCCGGGTGCGGTAATT | |
| | | GAAGATCTGTCCGGTCTGCTGGATATGTTCATGCTGTGATAA | |
| 413- | E41D, S59T, Y89F, | ATGATCAAGTATAAAGCCGTGTTTTTCGACTTCGACTATACGTTG | 58 |
| 11 | D124H, A140T | GCCGATAGCAGCAAAGCTGTTATTGAGTGCATCAATTATGCGCT | |
| | H142P, K101Q, | GCAGAAAATGGGTTACCCGGAGAGCAGCCCGGACTCTATTTGTC | |
| | D119A, V134I | GTACCATTGGCCTGACGTTGGCAGAGGCATACAAAATCCTGACC | |
| | | GGCGATACCAGCGACAGCAACGCAGATCTGTTTGCGCAATACTT | |
| | | TAAGGAACGTGCGGATCTGGTTATGTGTGACCGCACCGTTATGT | |
| | | TCAGCACCGTTGAGTGCGTCCTGAAGAAACTGAAACAGGCCGAT | |
| | | GTTAAGACCGGTATTGTCTCCACGAAATACCGCTACCGCATCGA | |
| | | AGCGATCTTAAAGCGTCACAAGCTGCTCCAGTATTTTGACGTCA | |
| | | TCATCGGTGGCGAAGATGTGACCGCGCCGAAGCCAGACCCGGA | |
| | | AGGCCTGCTGAAGGCAATTAGCATGGTCGGTTGCCAAAAGGAA | |
| | | GAGGTTCTGTTCGTTGGTGACAGCACTGTGGACGCGCGTACGGC | |
| | | GAAAAACGCTGGCGTGGACTTTGTGGCAGTCTTGACCGGCACCA | |
| | | CCGGTGCGAATGAATTCTCGGAGTACAACCCGGGTGCGGTAATT | |
| | | GAAGATCTGTCCGGTCTGCTGGATATGTTCATGCTGTGATAA | |
| 413- | E41D, S59T, Y89F, | ATGATCAAGTATAAAGCCGTGTTTTTCGACTTCGACTATACGTTG | 59 |
| 12 | D124H, A140T | GCCGATAGCAGCAAAGCTGTTATTGAGTGCATCAATTATGCGCT | |
| | H142P, K101Q, | GCAGAAAATGGGTTACCCGGAGAGCAGCCCGGACTCTATTTGTC | |
| | D119A, E137D | GTACCATTGGCCTGACGTTGGCAGAGGCATACAAAATCCTGACC | |
| | | GGCGATACCAGCGACAGCAACGCAGATCTGTTTGCGCAATACTT | |
| | | TAAGGAACGTGCGGATCTGGTTATGTGTGACCGCACCGTTATGT | |
| | | TCAGCACCGTTGAGTGCGTCCTGAAGAAACTGAAACAGGCCGAT | |
| | | GTTAAGACCGGTATTGTCTCCACGAAATACCGCTACCGCATCGA | |
| | | AGCGATCTTAAAGCGTCACAAGCTGCTCCAGTATTTTGACGTCA | |
| | | TCGTGGGTGGCGACGATGTGACCGCGCCGAAGCCAGACCCGGA | |
| | | AGGCCTGCTGAAGGCAATTAGCATGGTCGGTTGCCAAAAGGAA | |
| | | GAGGTTCTGTTCGTTGGTGACAGCACTGTGGACGCGCGTACGGC | |
| | | GAAAAACGCTGGCGTGGACTTTGTGGCAGTCTTGACCGGCACCA | |
| | | CCGGTGCGAATGAATTCTCGGAGTACAACCCGGGTGCGGTAATT | |
| | | GAAGATCTGTCCGGTCTGCTGGATATGTTCATGCTGTGATAA | |
| 413- | E41D, S59T, Y89F, | ATGATCAAGTATAAAGCCGTGTTTTTCGACTTCGACTATACGTTG | 60 |
| 13 | D124H, A140T | GCCGATAGCAGCAAAGCTGTTATTGAGTGCATCAATTATGCGCT | |
| | H142P, D119A, | GCAGAAAATGGGTTACCCGGAGAGCAGCCCGGACTCTATTTGTC | |
| | V134I, E137D | GTACCATTGGCCTGACGTTGGCAGAGGCATACAAAATCCTGACC | |
| | | GGCGATACCAGCGACAGCAACGCAGATCTGTTTGCGCAATACTT | |
| | | TAAGGAACGTGCGGATCTGGTTATGTGTGACCGCACCGTTATGT | |
| | | TCAGCACCGTTGAGTGCGTCCTGAAGAAACTGAAAAAAGCCGAT | |
| | | GTTAAGACCGGTATTGTCTCCACGAAATACCGCTACCGCATCGA | |
| | | AGCGATCTTAAAGCGTCACAAGCTGCTCCAGTATTTTGACGTCA | |
| | | TCATCGGTGGCGACGATGTGACCGCGCCGAAGCCAGACCCGGA | |
| | | AGGCCTGCTGAAGGCAATTAGCATGGTCGGTTGCCAAAAGGAA | |
| | | GAGGTTCTGTTCGTTGGTGACAGCACTGTGGACGCGCGTACGGC | |
| | | GAAAAACGCTGGCGTGGACTTTGTGGCAGTCTTGACCGGCACCA | |
| | | CCGGTGCGAATGAATTCTCGGAGTACAACCCGGGTGCGGTAATT | |
| | | GAAGATCTGTCCGGTCTGCTGGATATGTTCATGCTGTGATAA | |
| 413- | E41D, S59T, Y89F, | ATGATCAAGTATAAAGCCGTGTTTTTCGACTTCGACTATACGTTG | 61 |
| 14 | D124H, A140T | GCCGATAGCAGCAAAGCTGTTATTGAGTGCATCAATTATGCGCT | |
| | H142P, K101Q, | GCAGAAAATGGGTTACCCGGAGAGCAGCCCGGACTCTATTTGTC | |
| | V134I, E137D | GTACCATTGGCCTGACGTTGGCAGAGGCATACAAAATCCTGACC | |
| | | GGCGATACCAGCGACAGCAACGCAGATCTGTTTGCGCAATACTT | |
| | | TAAGGAACGTGCGGATCTGGTTATGTGTGACCGCACCGTTATGT | |
| | | TCAGCACCGTTGAGTGCGTCCTGAAGAAACTGAAACAGGCCGAT | |
| | | GTTAAGACCGGTATTGTCTCCACGAAATACCGCTACCGCATCGA | |
| | | AGATATCTTAAAGCGTCACAAGCTGCTCCAGTATTTTGACGTCA | |
| | | TCATCGGTGGCGACGATGTGACCGCGCCGAAGCCAGACCCGGA | |
| | | AGGCCTGCTGAAGGCAATTAGCATGGTCGGTTGCCAAAAGGAA | |
| | | GAGGTTCTGTTCGTTGGTGACAGCACTGTGGACGCGCGTACGGC | |
| | | GAAAAACGCTGGCGTGGACTTTGTGGCAGTCTTGACCGGCACCA | |
| | | CCGGTGCGAATGAATTCTCGGAGTACAACCCGGGTGCGGTAATT | |
| | | GAAGATCTGTCCGGTCTGCTGGATATGTTCATGCTGTGATAA | |
| 413- | E41D, S59T, Y89F, | ATGATCAAGTATAAAGCCGTGTTTTTCGACTTCGACTATACGTTG | 62 |
| 15 | D124H, A140T | GCCGATAGCAGCAAAGCTGTTATTGAGTGCATCAATTATGCGCT | |
| | H142P, K101Q, | GCAGAAAATGGGTTACCCGGAGAGCAGCCCGGACTCTATTTGTC | |
| | D119A, V134I, | GTACCATTGGCCTGACGTTGGCAGAGGCATACAAAATCCTGACC | |
| | E137D | GGCGATACCAGCGACAGCAACGCAGATCTGTTTGCGCAATACTT | |
| | | TAAGGAACGTGCGGATCTGGTTATGTGTGACCGCACCGTTATGT | |
| | | TCAGCACCGTTGAGTGCGTCCTGAAGAAACTGAAACAGGCCGAT | |
| | | GTTAAGACCGGTATTGTCTCCACGAAATACCGCTACCGCATCGA | |
| | | AGCGATCTTAAAGCGTCACAAGCTGCTCCAGTATTTTGACGTCA | |
| | | TCATCGGTGGCGACGATGTGACCGCGCCGAAGCCAGACCCGGA | |
| | | AGGCCTGCTGAAGGCAATTAGCATGGTCGGTTGCCAAAAGGAA | |
| | | GAGGTTCTGTTCGTTGGTGACAGCACTGTGGACGCGCGTACGGC | |
| | | GAAAAACGCTGGCGTGGACTTTGTGGCAGTCTTGACCGGCACCA | |
| | | CCGGTGCGAATGAATTCTCGGAGTACAACCCGGGTGCGGTAATT | |
| | | GAAGATCTGTCCGGTCTGCTGGATATGTTCATGCTGTGATAA | |
| 446-0 | E41D, F55Y, S59T, | ATGATCAAGTATAAAGCCGTGTTTTTCGACTTCGACTATACGTTG | 63 |
| | S65A, R71A, Y89F, | GCCGATAGCAGCAAAGCTGTTATTGAGTGCATCAATTATGCGCT | |
| | K101Q, D124H, | GCAGAAAATGGGTTACCCGGAGAGCAGCCCGGACTCTATTTGTC | |
| | E137D, A140T, | GTACCATTGGCCTGACGTTGGCAGAGGCATACAAAATCCTGACC | |
| | H142P | GGCGATACCAGCGACGCGAACGCAGATCTGTTTGCGCAATACTT | |
| | | TAAGGAACGTGCGGATCTGGTTATGTGTGACCGCACCGTTATGT | |
| | | TCAGCACCGTTGAGTGCGTCCTGAAGAAACTGAAACAGGCCGAT | |
| | | GTTAAGACCGGTATTGTCTCCACGAAATACCGCTACCGCATCGA | |
| | | AGATATCTTAAAGCGTCACAAGCTGCTCCAGTATTTTGACGTCA | |
| | | TCGTGGGTGGCGACGATGTGACCGCGCCGAAGCCAGACCCGGA | |
| | | AGGCCTGCTGAAGGCAATTAGCATGGTCGGTTGCCAAAAGGAA | |
| | | GAGGTTCTGTTCGTTGGTGACAGCACTGTGGACGCGCGTACGGC | |
| | | GAAAAACGCTGGCGTGGACTTTGTGGCAGTCTTGACCGGCACCA | |
| | | CCGGTGCGAATGAATTCTCGGAGTACAACCCGGGTGCGGTAATT | |
| | | GAGGATCTGTCCGGTCTGCTGGATATGTTCATGCTGTGATAA | |
| 446-5 | E41D, F55Y, S59T, | ATGATCAAGTATAAAGCCGTGTTTTTCGACTTCGACTATACGTTG | 64 |
| | S65A, R71A, Y89F, | GCCGATAGCAGCAAAGCTGTTATTGAGTGCATCAATTATGCGCT | |
| | K101Q, D124H, | GCAGAAAATGGGTTACCCGGAGAGCAGCCCGGACTCTATTTGTC | |
| | E137D, A140T, | GTACCATTGGCCTGACGTTGGCAGAGGCATACAAAATCCTGACC | |
| | H142P, D119A | GGCGATACCAGCGACGCGAACGCAGATCTGTTTGCGCAATACTT | |
| | | TAAGGAACGTGCGGATCTGGTTATGTGTGACCGCACCGTTATGT | |
| | | TCAGCACCGTTGAGTGCGTCCTGAAGAAACTGAAACAGGCCGAT | |
| | | GTTAAGACCGGTATTGTCTCCACGAAATACCGCTACCGCATCGA | |
| | | AGCGATCTTAAAGCGTCACAAGCTGCTCCAGTATTTTGACGTCA | |
| | | TCGTGGGTGGCGACGATGTGACCGCGCCGAAGCCAGACCCGGA | |
| | | AGGCCTGCTGAAGGCAATTAGCATGGTCGGTTGCCAAAAGGAA | |
| | | GAGGTTCTGTTCGTTGGTGACAGCACTGTGGACGCGCGTACGGC | |
| | | GAAAAACGCTGGCGTGGACTTTGTGGCAGTCTTGACCGGCACCA | |
| | | CCGGTGCGAATGAATTCTCGGAGTACAACCCGGGTGCGGTAATT | |
| | | GAAGATCTGTCCGGTCTGCTGGATATGTTCATGCTGTGATAA | |
| 446-6 | E41D, F55Y, S59T, | ATGATCAAGTATAAAGCCGTGTTTTTCGACTTCGACTATACGTTG | 65 |
| | S65A, R71A, Y89F, | GCCGATAGCAGCAAAGCTGTTATTGAGTGCATCAATTATGCGCT | |
| | K101Q, D124H, | GCAGAAAATGGGTTACCCGGAGAGCAGCCCGGACTCTATTTGTC | |
| | E137D, A140T, | GTACCATTGGCCTGACGTTGGCAGAGGCATACAAAATCCTGACC | |
| | H142P, V134I | GGCGATACCAGCGACGCGAACGCAGATCTGTTTGCGCAATACTT | |
| | | TAAGGAACGTGCGGATCTGGTTATGTGTGACCGCACCGTTATGT | |
| | | TCAGCACCGTTGAGTGCGTCCTGAAGAAACTGAAACAGGCCGAT | |
| | | GTTAAGACCGGTATTGTCTCCACGAAATACCGCTACCGCATCGA | |
| | | AGATATCTTAAAGCGTCACAAGCTGCTCCAGTATTTTGACGTCA | |
| | | TCATCGGTGGCGACGATGTGACCGCGCCGAAGCCAGACCCGGA | |
| | | AGGCCTGCTGAAGGCAATTAGCATGGTCGGTTGCCAAAAGGAA | |
| | | GAGGTTCTGTTCGTTGGTGACAGCACTGTGGACGCGCGTACGGC | |
| | | GAAAAACGCTGGCGTGGACTTTGTGGCAGTCTTGACCGGCACCA | |
| | | CCGGTGCGAATGAATTCTCGGAGTACAACCCGGGTGCGGTAATT | |
| | | GAAGATCTGTCCGGTCTGCTGGATATGTTCATGCTGTGATAA | |
| 446-7 | E41D, F55Y, S59T, | ATGATCAAGTATAAAGCCGTGTTTTTCGACTTCGACTATACGTTG | 66 |
| | S65A, R71A, Y89F, | GCCGATAGCAGCAAAGCTGTTATTGAGTGCATCAATTATGCGCT | |
| | K101Q, D124H, | GCAGAAAATGGGTTACCCGGAGAGCAGCCCGGACTCTATTTGTC | |
| | E137D, A140T, | GTACCATTGGCCTGACGTTGGCAGAGGCATACAAAATCCTGACC | |
| | H142P, D119A, | GGCGATACCAGCGACGCGAACGCAGATCTGTTTGCGCAATACTT | |
| | V134I | TAAGGAACGTGCGGATCTGGTTATGTGTGACCGCACCGTTATGT | |
| | | TCAGCACCGTTGAGTGCGTCCTGAAGAAACTGAAACAGGCCGAT | |
| | | GTTAAGACCGGTATTGTCTCCACGAAATACCGCTACCGCATCGA | |
| | | AGCGATCTTAAAGCGTCACAAGCTGCTCCAGTATTTTGACGTCA | |
| | | TCATCGGTGGCGACGATGTGACCGCGCCGAAGCCAGACCCGGA | |
| | | AGGCCTGCTGAAGGCAATTAGCATGGTCGGTTGCCAAAAGGAA | |
| | | GAGGTTCTGTTCGTTGGTGACAGCACTGTGGACGCGCGTACGGC | |
| | | GAAAAACGCTGGCGTGGACTTTGTGGCAGTCTTGACCGGCACCA | |
| | | CCGGTGCGAATGAATTCTCGGAGTACAACCCGGGTGCGGTAATT | |
| | | GAAGATCTGTCCGGTCTGCTGGATATGTTCATGCTGTGATAA | |
| 375 | S38C, E41D, T50S, | ATGATCAAGTATAAAGCCGTGTTTTTCGACTTCGACTATACGTTG | 67 |
| | S59T, D119A, | GCCGATAGCAGCAAAGCTGTTATTGAGTGCATCAATTATGCGCT | |
| | D124H, A140T, | GCAGAAAATGGGTTACCCGGAGTGCAGCCCGGACTCTATTTGTC | |
| | H142P, E206P | GTACCATTGGCCTGAGCTTGGCAGAGGCATTCAAAATCCTGACC | |
| | | GGCGATACCAGCGACAGCAACGCAGATCTGTTTCGTCAATACTT | |
| | | TAAGGAACGTGCGGATCTGGTTATGTGTGACCGCACCGTTATGT | |
| | | ACAGCACCGTTGAGTGCGTCCTGAAGAAACTGAAAAAAGCCGA | |
| | | TGTTAAGACCGGTATTGTCTCCACGAAATACCGCTACCGCATCG | |
| | | AAGCGATCTTAAAGCGTCACAAGCTGCTCCAGTATTTTGACGTC | |
| | | ATCGTGGGTGGCGAAGATGTGACCGCGCCGAAGCCAGACCCGG | |
| | | AAGGCCTGCTGAAGGCAATTAGCATGGTCGGTTGCCAAAAGGA | |
| | | AGAGGTTCTGTTCGTTGGTGACAGCACTGTGGACGCGCGTACGG | |
| | | CGAAAAACGCTGGCGTGGACTTTGTGGCAGTCTTGACCGGCACC | |
| | | ACCGGTGCGAATGAATTCTCGGAGTACAACCCGGGTGCGGTAAT | |
| | | TCCGGATCTGTCCGGTCTGCTGGATATGTTCATGCTGTAA | |
| 377 | S38C, E41D, D119A, | ATGATCAAGTATAAAGCCGTGTTTTTCGACTTCGACTATACGTTG | 68 |
| | D124H, A140T, | GCCGATAGCAGCAAAGCTGTTATTGAGTGCATCAATTATGCGCT | |
| | H142P, L150N, | GCAGAAAATGGGTTACCCGGAGTGCAGCCCGGACTCTATTTGTC | |
| | S197A, E206P | GTACCATTGGCCTGACGTTGGCAGAGGCATTCAAAATCCTGAGC | |
| | | GGCGATACCAGCGACAGCAACGCAGATCTGTTTCGTCAATACTT | |
| | | TAAGGAACGTGCGGATCTGGTTATGTGTGACCGCACCGTTATGT | |
| | | ACAGCACCGTTGAGTGCGTCCTGAAGAAACTGAAAAAAGCCGA | |
| | | TGTTAAGACCGGTATTGTCTCCACGAAATACCGCTACCGCATCG | |
| | | AAGCGATCTTAAAGCGTCACAAGCTGCTCCAGTATTTTGACGTC | |
| | | ATCGTGGGTGGCGAAGATGTGACCGCGCCGAAGCCAGACCCGG | |
| | | AAGGCCTGAACAAGGCAATTAGCATGGTCGGTTGCCAAAAGGA | |
| | | AGAGGTTCTGTTCGTTGGTGACAGCACTGTGGACGCGCGTACGG | |
| | | CGAAAAACGCTGGCGTGGACTTTGTGGCAGTCTTGACCGGCACC | |
| | | ACCGGTGCGAATGAATTCGCGGAGTACAACCCGGGTGCGGTAAT | |
| | | TCCGGATCTGTCCGGTCTGCTGGATATGTTCATGCTGTAA | |
| 382 | S38C, E41D, S59T, | ATGATCAAGTATAAAGCCGTGTTTTTCGACTTCGACTATACGTTG | 69 |
| | R71A, Y89F, | GCCGATAGCAGCAAAGCTGTTATTGAGTGCATCAATTATGCGCT | |
| | D124H, A140T, | GCAGAAAATGGGTTACCCGGAGTGCAGCCCGGACTCTATTTGTC | |
| | H142P, E206P | GTACCATTGGCCTGACGTTGGCAGAGGCATTCAAAATCCTGACC | |
| | | GGCGATACCAGCGACAGCAACGCAGATCTGTTTGCGCAATACTT | |
| | | TAAGGAACGTGCGGATCTGGTTATGTGTGACCGCACCGTTATGT | |
| | | TCAGCACCGTTGAGTGCGTCCTGAAGAAACTGAAAAAAGCCGAT | |
| | | GTTAAGACCGGTATTGTCTCCACGAAATACCGCTACCGCATCGA | |
| | | AGATATCTTAAAGCGTCACAAGCTGCTCCAGTATTTTGACGTCA | |
| | | TCGTGGGTGGCGAAGATGTGACCGCGCCGAAGCCAGACCCGGA | |
| | | AGGCCTGCTGAAGGCAATTAGCATGGTCGGTTGCCAAAAGGAA | |
| | | GAGGTTCTGTTCGTTGGTGACAGCACTGTGGACGCGCGTACGGC | |
| | | GAAAAACGCTGGCGTGGACTTTGTGGCAGTCTTGACCGGCACCA | |
| | | CCGGTGCGAATGAATTCTCGGAGTACAACCCGGGTGCGGTAATT | |
| | | CCGGATCTGTCCGGTCTGCTGGATATGTTCATGCTGTAA | |

TABLE 6
Allulose 6-phosphate phosphatases (A6PPs) with hexahistidine purification tags-
Nucleic acid sequences

| | A6PP amino acid | | |
|---|---|---|---|
| | substitutions relative | | |
| | to wild-type (SEQ ID | | SEQ ID |
| | NO: 1) | Nucleic Acid Sequence | NO: |
| Wild- | | ATGCATCACCATCACCACCACATCAAATACAAAGCCGTGTTTTT | 70 |
| type | | CGATTTCGATTATACCCTGGCAGATAGCAGCAAAGCAGTTATTG | |
| | | AATGTATTAACTACGCCCTGCAGAAAATGGGTTATCCGGAAAGC | |
| | | TCACCGGAAAGCATTTGTCGTACCATTGGTCTGACCCTGGCCGA | |
| | | AGCATTCAAAATTCTGAGCGGTGATACCAGCGATAGCAATGCAG | |
| | | ACCTGTTTCGTCAGTATTTCAAAGAACGTGCAGATCTGGTTATGT | |
| | | GTGATCGTACCGTTATGTATAGCACCGTTGAATGTGTTCTGAAA | |
| | | AAGCTGAAAAAAGCCGATGTGAAAACCGGTATTGTGAGCACCA | |
| | | AATATCGTTATCGCATTGAGGATATTCTGAAACGCGATAAACTG | |
| | | CTGCAGTATTTCGATGTTATTGTTGGTGGTGAAGATGTGGCAGC | |
| | | ACATAAACCGGATCCTGAAGGTCTGCTGAAAGCAATTAGCATGG | |
| | | TTGGTTGTCAGAAAGAAGAGGTTCTGTTTGTTGGTGATTCAACC | |
| | | GTTGATGCACGTACCGCAAAAAATGCCGGTGTTGATTTTGTTGC | |
| | | AGTTCTGACCGGCACCACCGGTGCAAATGAATTTTCTGAATATA | |
| | | ATCCGGGTGCCGTGATTGAAGATCTGAGTGGTCTGCTGGATATG | |
| | | TTTATGCTGTAA | |
| | E41D, S59T, Y89F, | ATGCACCACCATCATCATCACATCAAGTATAAAGCCGTGTTTTTC | 71 |
| | D124H, A140T | GACTTCGACTATACGTTGGCCGATAGCAGCAAAGCTGTTATTGA | |
| | H142P | GTGCATCAATTATGCGCTGCAGAAAATGGGTTACCCGGAGAGCA | |
| | | GCCCGGAGTCTATTTGTCGTACCATTGGCCTGACGTTGGCAGAG | |
| | | GCATTCAAAATCCTGAGCGGCGATACCAGCGACAGCAACGCAG | |
| | | ATCTGTTTCGTCAATACTTTAAGGAACGTGCGGATCTGGTTATGT | |
| | | GTGACCGCACCGTTATGTACAGCACCGTTGAGTGCGTCCTGAAG | |
| | | AAACTGAAAAAAGCCGATGTTAAGACCGGTATTGTCTCCACGAA | |
| | | ATACCGCTACCGCATCGAAGATATCTTAAAGCGTGACAAGCTGC | |
| | | TCCAGTATTTTGACGTCATCGTGGGTGGCGAAGATGTGGCGGCG | |
| | | CACAAGCCAGACCCGGAAGGCCTGCTGAAGGCAATTAGCATGG | |
| | | TCGGTTGCCAAAAGGAAGAGGTTCTGTTCGTTGGTGACAGCACT | |
| | | GTGGACGCGCGTACGGCGAAAAACGCTGGCGTGGACTTTGTGGC | |
| | | AGTCTTGACCGGCACCACCGGTGCGAATGAATTCTCGGAGTACA | |
| | | ACCCGGGTGCGGTAATTGAAGATCTGTCCGGTCTGCTGGATATG | |
| | | TTCATGCTGTGATAA | |
| 413-0 | E41D, S59T, Y89F, | ATGCACCACCATCATCATCACATCAAGTATAAAGCCGTGTTTTTC | 72 |
| | D124H, A140T | GACTTCGACTATACGTTGGCCGATAGCAGCAAAGCTGTTATTGA | |
| | H142P, F55Y, R71A | GTGCATCAATTATGCGCTGCAGAAAATGGGTTACCCGGAGAGCA | |
| | | GCCCGGACTCTATTTGTCGTACCATTGGCCTGACGTTGGCAGAG | |
| | | GCATACAAAATCCTGACCGGCGATACCAGCGACAGCAACGCAG | |
| | | ATCTGTTTGCGCAATACTTTAAGGAACGTGCGGATCTGGTTATGT | |
| | | GTGACCGCACCGTTATGTTCAGCACCGTTGAGTGCGTCCTGAAG | |
| | | AAACTGAAAAAAGCCGATGTTAAGACCGGTATTGTCTCCACGAA | |
| | | ATACCGCTACCGCATCGAAGATATCTTAAAGCGTCACAAGCTGC | |
| | | TCCAGTATTTTGACGTCATCGTGGGTGGCGAAGATGTGACCGCG | |
| | | CCGAAGCCAGACCCGGAAGGCCTGCTGAAGGCAATTAGCATGG | |
| | | TCGGTTGCCAAAAGGAAGAGGTTCTGTTCGTTGGTGACAGCACT | |
| | | GTGGACGCGCGTACGGCGAAAAACGCTGGCGTGGACTTTGTGGC | |
| | | AGTCTTGACCGGCACCACCGGTGCGAATGAATTCTCGGAGTACA | |
| | | ACCCGGGTGCGGTAATTGAGGATCTGTCCGGTCTGCTGGATATG | |
| | | TTCATGCTGTGATAA | |
| 413-1 | E41D, S59T, Y89F, | ATGCACCACCATCATCATCACATCAAGTATAAAGCCGTGTTTTTC | 73 |
| | D124H, A140T | GACTTCGACTATACGTTGGCCGATAGCAGCAAAGCTGTTATTGA | |
| | H142P, F55Y, R71A, | GTGCATCAATTATGCGCTGCAGAAAATGGGTTACCCGGAGAGCA | |
| | D119A | GCCCGGACTCTATTTGTCGTACCATTGGCCTGACGTTGGCAGAG | |
| | | GCATACAAAATCCTGACCGGCGATACCAGCGACAGCAACGCAG | |
| | | ATCTGTTTGCGCAATACTTTAAGGAACGTGCGGATCTGGTTATGT | |
| | | GTGACCGCACCGTTATGTTCAGCACCGTTGAGTGCGTCCTGAAG | |
| | | AAACTGAAAAAAGCCGATGTTAAGACCGGTATTGTCTCCACGAA | |
| | | ATACCGCTACCGCATCGAAGCGATCTTAAAGCGTCACAAGCTGC | |
| | | TCCAGTATTTTGACGTCATCGTGGGTGGCGAAGATGTGACCGCG | |
| | | CCGAAGCCAGACCCGGAAGGCCTGCTGAAGGCAATTAGCATGG | |
| | | TCGGTTGCCAAAAGGAAGAGGTTCTGTTCGTTGGTGACAGCACT | |
| | | GTGGACGCGCGTACGGCGAAAAACGCTGGCGTGGACTTTGTGGC | |
| | | AGTCTTGACCGGCACCACCGGTGCGAATGAATTCTCGGAGTACA | |
| | | ACCCGGGTGCGGTAATTGAAGATCTGTCCGGTCTGCTGGATATG | |
| | | TTCATGCTGTGATAA | |
| 413-3 | E41D, S59T, Y89F, | ATGCACCACCATCATCATCACATCAAGTATAAAGCCGTGTTTTTC | 74 |
| | D124H, A140T | GACTTCGACTATACGTTGGCCGATAGCAGCAAAGCTGTTATTGA | |
| | H142P, V134I | GTGCATCAATTATGCGCTGCAGAAAATGGGTTACCCGGAGAGCA | |
| | | GCCCGGACTCTATTTGTCGTACCATTGGCCTGACGTTGGCAGAG | |
| | | GCATACAAAATCCTGACCGGCGATACCAGCGACAGCAACGCAG | |
| | | ATCTGTTTGCGCAATACTTTAAGGAACGTGCGGATCTGGTTATGT | |
| | | GTGACCGCACCGTTATGTTCAGCACCGTTGAGTGCGTCCTGAAG | |
| | | AAACTGAAAAAAGCCGATGTTAAGACCGGTATTGTCTCCACGAA | |
| | | ATACCGCTACCGCATCGAAGATATCTTAAAGCGTCACAAGCTGC | |
| | | TCCAGTATTTTGACGTCATCATCGGTGGCGAAGATGTGACCGCG | |
| | | CCGAAGCCAGACCCGGAAGGCCTGCTGAAGGCAATTAGCATGG | |
| | | TCGGTTGCCAAAAGGAAGAGGTTCTGTTCGTTGGTGACAGCACT | |
| | | GTGGACGCGCGTACGGCGAAAAACGCTGGCGTGGACTTTGTGGC | |
| | | AGTCTTGACCGGCACCACCGGTGCGAATGAATTCTCGGAGTACA | |
| | | ACCCGGGTGCGGTAATTGAAGATCTGTCCGGTCTGCTGGATATG | |
| | | TTCATGCTGTGATAA | |
| 413-4 | E41D, S59T, Y89F, | ATGCACCACCATCATCATCACATCAAGTATAAAGCCGTGTTTTTC | 75 |
| | D124H, A140T | GACTTCGACTATACGTTGGCCGATAGCAGCAAAGCTGTTATTGA | |
| | H142P, E137D | GTGCATCAATTATGCGCTGCAGAAAATGGGTTACCCGGAGAGCA | |
| | | GCCCGGACTCTATTTGTCGTACCATTGGCCTGACGTTGGCAGAG | |
| | | GCATACAAAATCCTGACCGGCGATACCAGCGACAGCAACGCAG | |
| | | ATCTGTTTGCGCAATACTTTAAGGAACGTGCGGATCTGGTTATGT | |
| | | GTGACCGCACCGTTATGTTCAGCACCGTTGAGTGCGTCCTGAAG | |
| | | AAACTGAAAAAAGCCGATGTTAAGACCGGTATTGTCTCCACGAA | |
| | | ATACCGCTACCGCATCGAAGATATCTTAAAGCGTCACAAGCTGC | |
| | | TCCAGTATTTTGACGTCATCGTGGGTGGCGACGATGTGACCGCG | |
| | | CCGAAGCCAGACCCGGAAGGCCTGCTGAAGGCAATTAGCATGG | |
| | | TCGGTTGCCAAAAGGAAGAGGTTCTGTTCGTTGGTGACAGCACT | |
| | | GTGGACGCGCGTACGGCGAAAAACGCTGGCGTGGACTTTGTGGC | |
| | | AGTCTTGACCGGCACCACCGGTGCGAATGAATTCTCGGAGTACA | |
| | | ACCCGGGTGCGGTAATTGAAGATCTGTCCGGTCTGCTGGATATG | |
| | | TTCATGCTGTGATAA | |
| 413-5 | E41D, S59T, Y89F, | ATGCACCACCATCATCATCACATCAAGTATAAAGCCGTGTTTTTC | 76 |
| | D124H, A140T | GACTTCGACTATACGTTGGCCGATAGCAGCAAAGCTGTTATTGA | |
| | H142P, K101Q, | GTGCATCAATTATGCGCTGCAGAAAATGGGTTACCCGGAGAGCA | |
| | D119A | GCCCGGACTCTATTTGTCGTACCATTGGCCTGACGTTGGCAGAG | |
| | | GCATACAAAATCCTGACCGGCGATACCAGCGACAGCAACGCAG | |
| | | ATCTGTTTGCGCAATACTTTAAGGAACGTGCGGATCTGGTTATGT | |
| | | GTGACCGCACCGTTATGTTCAGCACCGTTGAGTGCGTCCTGAAG | |
| | | AAACTGAAACAGGCCGATGTTAAGACCGGTATTGTCTCCACGAA | |
| | | ATACCGCTACCGCATCGAAGCGATCTTAAAGCGTCACAAGCTGC | |
| | | TCCAGTATTTTGACGTCATCGTGGGTGGCGAAGATGTGACCGCG | |
| | | CCGAAGCCAGACCCGGAAGGCCTGCTGAAGGCAATTAGCATGG | |
| | | TCGGTTGCCAAAAGGAAGAGGTTCTGTTCGTTGGTGACAGCACT | |
| | | GTGGACGCGCGTACGGCGAAAAACGCTGGCGTGGACTTTGTGGC | |
| | | AGTCTTGACCGGCACCACCGGTGCGAATGAATTCTCGGAGTACA | |
| | | ACCCGGGTGCGGTAATTGAAGATCTGTCCGGTCTGCTGGATATG | |
| | | TTCATGCTGTGATAA | |
| 413-6 | E41D, S59T, Y89F, | ATGCACCACCATCATCATCACATCAAGTATAAAGCCGTGTTTTTC | 77 |
| | D124H, A140T | GACTTCGACTATACGTTGGCCGATAGCAGCAAAGCTGTTATTGA | |
| | H142P, D119A, | GTGCATCAATTATGCGCTGCAGAAAATGGGTTACCCGGAGAGCA | |
| | V134I | GCCCGGACTCTATTTGTCGTACCATTGGCCTGACGTTGGCAGAG | |
| | | GCATACAAAATCCTGACCGGCGATACCAGCGACAGCAACGCAG | |
| | | ATCTGTTTGCGCAATACTTTAAGGAACGTGCGGATCTGGTTATGT | |
| | | GTGACCGCACCGTTATGTTCAGCACCGTTGAGTGCGTCCTGAAG | |
| | | AAACTGAAAAAAGCCGATGTTAAGACCGGTATTGTCTCCACGAA | |
| | | ATACCGCTACCGCATCGAAGCGATCTTAAAGCGTCACAAGCTGC | |
| | | TCCAGTATTTTGACGTCATCATCGGTGGCGAAGATGTGACCGCG | |
| | | CCGAAGCCAGACCCGGAAGGCCTGCTGAAGGCAATTAGCATGG | |
| | | TCGGTTGCCAAAAGGAAGAGGTTCTGTTCGTTGGTGACAGCACT | |
| | | GTGGACGCGCGTACGGCGAAAAACGCTGGCGTGGACTTTGTGGC | |
| | | AGTCTTGACCGGCACCACCGGTGCGAATGAATTCTCGGAGTACA | |
| | | ACCCGGGTGCGGTAATTGAAGATCTGTCCGGTCTGCTGGATATG | |
| | | TTCATGCTGTGATAA | |
| 413-7 | E41D, S59T, Y89F, | ATGCACCACCATCATCATCACATCAAGTATAAAGCCGTGTTTTTC | 78 |
| | D124H, A140T | GACTTCGACTATACGTTGGCCGATAGCAGCAAAGCTGTTATTGA | |
| | H142P, D119A, | GTGCATCAATTATGCGCTGCAGAAAATGGGTTACCCGGAGAGCA | |
| | E137D | GCCCGGACTCTATTTGTCGTACCATTGGCCTGACGTTGGCAGAG | |
| | | GCATACAAAATCCTGACCGGCGATACCAGCGACAGCAACGCAG | |
| | | ATCTGTTTGCGCAATACTTTAAGGAACGTGCGGATCTGGTTATGT | |
| | | GTGACCGCACCGTTATGTTCAGCACCGTTGAGTGCGTCCTGAAG | |
| | | AAACTGAAAAAAGCCGATGTTAAGACCGGTATTGTCTCCACGAA | |
| | | ATACCGCTACCGCATCGAAGCGATCTTAAAGCGTCACAAGCTGC | |
| | | TCCAGTATTTTGACGTCATCGTGGGTGGCGACGATGTGACCGCG | |
| | | CCGAAGCCAGACCCGGAAGGCCTGCTGAAGGCAATTAGCATGG | |
| | | TCGGTTGCCAAAAGGAAGAGGTTCTGTTCGTTGGTGACAGCACT | |
| | | GTGGACGCGCGTACGGCGAAAAACGCTGGCGTGGACTTTGTGGC | |
| | | AGTCTTGACCGGCACCACCGGTGCGAATGAATTCTCGGAGTACA | |
| | | ACCCGGGTGCGGTAATTGAAGATCTGTCCGGTCTGCTGGATATG | |
| | | TTCATGCTGTGATAA | |
| 413-8 | E41D, S59T, Y89F, | ATGCACCACCATCATCATCACATCAAGTATAAAGCCGTGTTTTTC | 79 |
| | D124H, A140T | GACTTCGACTATACGTTGGCCGATAGCAGCAAAGCTGTTATTGA | |
| | H142P, K101Q, | GTGCATCAATTATGCGCTGCAGAAAATGGGTTACCCGGAGAGCA | |
| | V134I | GCCCGGACTCTATTTGTCGTACCATTGGCCTGACGTTGGCAGAG | |
| | | GCATACAAAATCCTGACCGGCGATACCAGCGACAGCAACGCAG | |
| | | ATCTGTTTGCGCAATACTTTAAGGAACGTGCGGATCTGGTTATGT | |
| | | GTGACCGCACCGTTATGTTCAGCACCGTTGAGTGCGTCCTGAAG | |
| | | AAACTGAAACAGGCCGATGTTAAGACCGGTATTGTCTCCACGAA | |
| | | ATACCGCTACCGCATCGAAGATATCTTAAAGCGTCACAAGCTGC | |
| | | TCCAGTATTTTGACGTCATCATCGGTGGCGAAGATGTGACCGCG | |
| | | CCGAAGCCAGACCCGGAAGGCCTGCTGAAGGCAATTAGCATGG | |
| | | TCGGTTGCCAAAAGGAAGAGGTTCTGTTCGTTGGTGACAGCACT | |
| | | GTGGACGCGCGTACGGCGAAAAACGCTGGCGTGGACTTTGTGGC | |
| | | AGTCTTGACCGGCACCACCGGTGCGAATGAATTCTCGGAGTACA | |
| | | ACCCGGGTGCGGTAATTGAAGATCTGTCCGGTCTGCTGGATATG | |
| | | TTCATGCTGTGATAA | |
| 413-9 | E41D, S59T, Y89F, | ATGCACCACCATCATCATCACATCAAGTATAAAGCCGTGTTTTTC | 80 |
| | D124H, A140T | GACTTCGACTATACGTTGGCCGATAGCAGCAAAGCTGTTATTGA | |
| | H142P, K101Q, | GTGCATCAATTATGCGCTGCAGAAAATGGGTTACCCGGAGAGCA | |
| | E137D | GCCCGGACTCTATTTGTCGTACCATTGGCCTGACGTTGGCAGAG | |
| | | GCATACAAAATCCTGACCGGCGATACCAGCGACAGCAACGCAG | |
| | | ATCTGTTTGCGCAATACTTTAAGGAACGTGCGGATCTGGTTATGT | |
| | | GTGACCGCACCGTTATGTTCAGCACCGTTGAGTGCGTCCTGAAG | |
| | | AAACTGAAACAGGCCGATGTTAAGACCGGTATTGTCTCCACGAA | |
| | | ATACCGCTACCGCATCGAAGATATCTTAAAGCGTCACAAGCTGC | |
| | | TCCAGTATTTTGACGTCATCGTGGGTGGCGACGATGTGACCGCG | |
| | | CCGAAGCCAGACCCGGAAGGCCTGCTGAAGGCAATTAGCATGG | |
| | | TCGGTTGCCAAAAGGAAGAGGTTCTGTTCGTTGGTGACAGCACT | |
| | | GTGGACGCGCGTACGGCGAAAAACGCTGGCGTGGACTTTGTGGC | |
| | | AGTCTTGACCGGCACCACCGGTGCGAATGAATTCTCGGAGTACA | |
| | | ACCCGGGTGCGGTAATTGAAGATCTGTCCGGTCTGCTGGATATG | |
| | | TTCATGCTGTGATAA | |
| 413- | E41D, S59T, Y89F, | ATGCACCACCATCATCATCACATCAAGTATAAAGCCGTGTTTTTC | 81 |
| 10 | D124H, A140T | GACTTCGACTATACGTTGGCCGATAGCAGCAAAGCTGTTATTGA | |
| | H142P, V134I, | GTGCATCAATTATGCGCTGCAGAAAATGGGTTACCCGGAGAGCA | |
| | E137D | GCCCGGACTCTATTTGTCGTACCATTGGCCTGACGTTGGCAGAG | |
| | | GCATACAAAATCCTGACCGGCGATACCAGCGACAGCAACGCAG | |
| | | ATCTGTTTGCGCAATACTTTAAGGAACGTGCGGATCTGGTTATGT | |
| | | GTGACCGCACCGTTATGTTCAGCACCGTTGAGTGCGTCCTGAAG | |
| | | AAACTGAAAAAAGCCGATGTTAAGACCGGTATTGTCTCCACGAA | |
| | | ATACCGCTACCGCATCGAAGATATCTTAAAGCGTCACAAGCTGC | |
| | | TCCAGTATTTTGACGTCATCATCGGTGGCGACGATGTGACCGCG | |
| | | CCGAAGCCAGACCCGGAAGGCCTGCTGAAGGCAATTAGCATGG | |
| | | TCGGTTGCCAAAAGGAAGAGGTTCTGTTCGTTGGTGACAGCACT | |
| | | GTGGACGCGCGTACGGCGAAAAACGCTGGCGTGGACTTTGTGGC | |
| | | AGTCTTGACCGGCACCACCGGTGCGAATGAATTCTCGGAGTACA | |
| | | ACCCGGGTGCGGTAATTGAAGATCTGTCCGGTCTGCTGGATATG | |
| | | TTCATGCTGTGATAA | |
| 413- | E41D, S59T, Y89F, | ATGCACCACCATCATCATCACATCAAGTATAAAGCCGTGTTTTTC | 82 |
| 11 | D124H, A140T | GACTTCGACTATACGTTGGCCGATAGCAGCAAAGCTGTTATTGA | |
| | H142P, K101Q, | GTGCATCAATTATGCGCTGCAGAAAATGGGTTACCCGGAGAGCA | |
| | D119A, V134I | GCCCGGACTCTATTTGTCGTACCATTGGCCTGACGTTGGCAGAG | |
| | | GCATACAAAATCCTGACCGGCGATACCAGCGACAGCAACGCAG | |
| | | ATCTGTTTGCGCAATACTTTAAGGAACGTGCGGATCTGGTTATGT | |
| | | GTGACCGCACCGTTATGTTCAGCACCGTTGAGTGCGTCCTGAAG | |
| | | AAACTGAAACAGGCCGATGTTAAGACCGGTATTGTCTCCACGAA | |
| | | ATACCGCTACCGCATCGAAGCGATCTTAAAGCGTCACAAGCTGC | |
| | | TCCAGTATTTTGACGTCATCATCGGTGGCGAAGATGTGACCGCG | |
| | | CCGAAGCCAGACCCGGAAGGCCTGCTGAAGGCAATTAGCATGG | |
| | | TCGGTTGCCAAAAGGAAGAGGTTCTGTTCGTTGGTGACAGCACT | |
| | | GTGGACGCGCGTACGGCGAAAAACGCTGGCGTGGACTTTGTGGC | |
| | | AGTCTTGACCGGCACCACCGGTGCGAATGAATTCTCGGAGTACA | |
| | | ACCCGGGTGCGGTAATTGAAGATCTGTCCGGTCTGCTGGATATG | |
| | | TTCATGCTGTGATAA | |
| 413- | E41D, S59T, Y89F, | ATGCACCACCATCATCATCACATCAAGTATAAAGCCGTGTTTTTC | 83 |
| 12 | D124H, A140T | GACTTCGACTATACGTTGGCCGATAGCAGCAAAGCTGTTATTGA | |
| | H142P, K101Q, | GTGCATCAATTATGCGCTGCAGAAAATGGGTTACCCGGAGAGCA | |
| | D119A, E137D | GCCCGGACTCTATTTGTCGTACCATTGGCCTGACGTTGGCAGAG | |
| | | GCATACAAAATCCTGACCGGCGATACCAGCGACAGCAACGCAG | |
| | | ATCTGTTTGCGCAATACTTTAAGGAACGTGCGGATCTGGTTATGT | |
| | | GTGACCGCACCGTTATGTTCAGCACCGTTGAGTGCGTCCTGAAG | |
| | | AAACTGAAACAGGCCGATGTTAAGACCGGTATTGTCTCCACGAA | |
| | | ATACCGCTACCGCATCGAAGCGATCTTAAAGCGTCACAAGCTGC | |
| | | TCCAGTATTTTGACGTCATCGTGGGTGGCGACGATGTGACCGCG | |
| | | CCGAAGCCAGACCCGGAAGGCCTGCTGAAGGCAATTAGCATGG | |
| | | TCGGTTGCCAAAAGGAAGAGGTTCTGTTCGTTGGTGACAGCACT | |
| | | GTGGACGCGCGTACGGCGAAAAACGCTGGCGTGGACTTTGTGGC | |
| | | AGTCTTGACCGGCACCACCGGTGCGAATGAATTCTCGGAGTACA | |
| | | ACCCGGGTGCGGTAATTGAAGATCTGTCCGGTCTGCTGGATATG | |
| | | TTCATGCTGTGATAA | |
| 413- | E41D, S59T, Y89F, | ATGCACCACCATCATCATCACATCAAGTATAAAGCCGTGTTTTTC | 84 |
| 13 | D124H, A140T | GACTTCGACTATACGTTGGCCGATAGCAGCAAAGCTGTTATTGA | |
| | H142P, D119A, | GTGCATCAATTATGCGCTGCAGAAAATGGGTTACCCGGAGAGCA | |
| | V134I, E137D | GCCCGGACTCTATTTGTCGTACCATTGGCCTGACGTTGGCAGAG | |
| | | GCATACAAAATCCTGACCGGCGATACCAGCGACAGCAACGCAG | |
| | | ATCTGTTTGCGCAATACTTTAAGGAACGTGCGGATCTGGTTATGT | |
| | | GTGACCGCACCGTTATGTTCAGCACCGTTGAGTGCGTCCTGAAG | |
| | | AAACTGAAAAAAGCCGATGTTAAGACCGGTATTGTCTCCACGAA | |
| | | ATACCGCTACCGCATCGAAGCGATCTTAAAGCGTCACAAGCTGC | |
| | | TCCAGTATTTTGACGTCATCATCGGTGGCGACGATGTGACCGCG | |
| | | CCGAAGCCAGACCCGGAAGGCCTGCTGAAGGCAATTAGCATGG | |
| | | TCGGTTGCCAAAAGGAAGAGGTTCTGTTCGTTGGTGACAGCACT | |
| | | GTGGACGCGCGTACGGCGAAAAACGCTGGCGTGGACTTTGTGGC | |
| | | AGTCTTGACCGGCACCACCGGTGCGAATGAATTCTCGGAGTACA | |
| | | ACCCGGGTGCGGTAATTGAAGATCTGTCCGGTCTGCTGGATATG | |
| | | TTCATGCTGTGATAA | |
| 413- | E41D, S59T, Y89F, | ATGCACCACCATCATCATCACATCAAGTATAAAGCCGTGTTTTTC | 85 |
| 14 | D124H, A140T | GACTTCGACTATACGTTGGCCGATAGCAGCAAAGCTGTTATTGA | |
| | H142P, K101Q, | GTGCATCAATTATGCGCTGCAGAAAATGGGTTACCCGGAGAGCA | |
| | V134I, E137D | GCCCGGACTCTATTTGTCGTACCATTGGCCTGACGTTGGCAGAG | |
| | | GCATACAAAATCCTGACCGGCGATACCAGCGACAGCAACGCAG | |
| | | ATCTGTTTGCGCAATACTTTAAGGAACGTGCGGATCTGGTTATGT | |
| | | GTGACCGCACCGTTATGTTCAGCACCGTTGAGTGCGTCCTGAAG | |
| | | AAACTGAAACAGGCCGATGTTAAGACCGGTATTGTCTCCACGAA | |
| | | ATACCGCTACCGCATCGAAGATATCTTAAAGCGTCACAAGCTGC | |
| | | TCCAGTATTTTGACGTCATCATCGGTGGCGACGATGTGACCGCG | |
| | | CCGAAGCCAGACCCGGAAGGCCTGCTGAAGGCAATTAGCATGG | |
| | | TCGGTTGCCAAAAGGAAGAGGTTCTGTTCGTTGGTGACAGCACT | |
| | | GTGGACGCGCGTACGGCGAAAAACGCTGGCGTGGACTTTGTGGC | |
| | | AGTCTTGACCGGCACCACCGGTGCGAATGAATTCTCGGAGTACA | |
| | | ACCCGGGTGCGGTAATTGAAGATCTGTCCGGTCTGCTGGATATG | |
| | | TTCATGCTGTGATAA | |
| 413- | E41D, S59T, Y89F, | ATGCACCACCATCATCATCACATCAAGTATAAAGCCGTGTTTTTC | 86 |
| 15 | D124H, A140T | GACTTCGACTATACGTTGGCCGATAGCAGCAAAGCTGTTATTGA | |
| | H142P, K101Q, | GTGCATCAATTATGCGCTGCAGAAAATGGGTTACCCGGAGAGCA | |
| | D119A, V134I, | GCCCGGACTCTATTTGTCGTACCATTGGCCTGACGTTGGCAGAG | |
| | E137D | GCATACAAAATCCTGACCGGCGATACCAGCGACAGCAACGCAG | |
| | | ATCTGTTTGCGCAATACTTTAAGGAACGTGCGGATCTGGTTATGT | |
| | | GTGACCGCACCGTTATGTTCAGCACCGTTGAGTGCGTCCTGAAG | |
| | | AAACTGAAACAGGCCGATGTTAAGACCGGTATTGTCTCCACGAA | |
| | | ATACCGCTACCGCATCGAAGCGATCTTAAAGCGTCACAAGCTGC | |
| | | TCCAGTATTTTGACGTCATCATCGGTGGCGACGATGTGACCGCG | |
| | | CCGAAGCCAGACCCGGAAGGCCTGCTGAAGGCAATTAGCATGG | |
| | | TCGGTTGCCAAAAGGAAGAGGTTCTGTTCGTTGGTGACAGCACT | |
| | | GTGGACGCGCGTACGGCGAAAAACGCTGGCGTGGACTTTGTGGC | |
| | | AGTCTTGACCGGCACCACCGGTGCGAATGAATTCTCGGAGTACA | |
| | | ACCCGGGTGCGGTAATTGAAGATCTGTCCGGTCTGCTGGATATG | |
| | | TTCATGCTGTGATAA | |
| 446-0 | E41D, F55Y, S59T, | ATGCACCACCATCATCATCACATCAAGTATAAAGCCGTGTTTTTC | 87 |
| | S65A, R71A, Y89F, | GACTTCGACTATACGTTGGCCGATAGCAGCAAAGCTGTTATTGA | |
| | K101Q, D124H, | GTGCATCAATTATGCGCTGCAGAAAATGGGTTACCCGGAGAGCA | |
| | E137D, A140T, | GCCCGGACTCTATTTGTCGTACCATTGGCCTGACGTTGGCAGAG | |
| | H142P | GCATACAAAATCCTGACCGGCGATACCAGCGACGCGAACGCAG | |
| | | ATCTGTTTGCGCAATACTTTAAGGAACGTGCGGATCTGGTTATGT | |
| | | GTGACCGCACCGTTATGTTCAGCACCGTTGAGTGCGTCCTGAAG | |
| | | AAACTGAAACAGGCCGATGTTAAGACCGGTATTGTCTCCACGAA | |
| | | ATACCGCTACCGCATCGAAGATATCTTAAAGCGTCACAAGCTGC | |
| | | TCCAGTATTTTGACGTCATCGTGGGTGGCGACGATGTGACCGCG | |
| | | CCGAAGCCAGACCCGGAAGGCCTGCTGAAGGCAATTAGCATGG | |
| | | TCGGTTGCCAAAAGGAAGAGGTTCTGTTCGTTGGTGACAGCACT | |
| | | GTGGACGCGCGTACGGCGAAAAACGCTGGCGTGGACTTTGTGGC | |
| | | AGTCTTGACCGGCACCACCGGTGCGAATGAATTCTCGGAGTACA | |
| | | ACCCGGGTGCGGTAATTGAGGATCTGTCCGGTCTGCTGGATATG | |
| | | TTCATGCTGTGATAA | |
| 446-5 | E41D, F55Y, S59T, | ATGCACCACCATCATCATCACATCAAGTATAAAGCCGTGTTTTTC | 88 |
| | S65A, R71A, Y89F, | GACTTCGACTATACGTTGGCCGATAGCAGCAAAGCTGTTATTGA | |
| | K101Q, D124H, | GTGCATCAATTATGCGCTGCAGAAAATGGGTTACCCGGAGAGCA | |
| | E137D, A140T, | GCCCGGACTCTATTTGTCGTACCATTGGCCTGACGTTGGCAGAG | |
| | H142P, D119A | GCATACAAAATCCTGACCGGCGATACCAGCGACGCGAACGCAG | |
| | | ATCTGTTTGCGCAATACTTTAAGGAACGTGCGGATCTGGTTATGT | |
| | | GTGACCGCACCGTTATGTTCAGCACCGTTGAGTGCGTCCTGAAG | |
| | | AAACTGAAACAGGCCGATGTTAAGACCGGTATTGTCTCCACGAA | |
| | | ATACCGCTACCGCATCGAAGCGATCTTAAAGCGTCACAAGCTGC | |
| | | TCCAGTATTTTGACGTCATCGTGGGTGGCGACGATGTGACCGCG | |
| | | CCGAAGCCAGACCCGGAAGGCCTGCTGAAGGCAATTAGCATGG | |
| | | TCGGTTGCCAAAAGGAAGAGGTTCTGTTCGTTGGTGACAGCACT | |
| | | GTGGACGCGCGTACGGCGAAAAACGCTGGCGTGGACTTTGTGGC | |
| | | AGTCTTGACCGGCACCACCGGTGCGAATGAATTCTCGGAGTACA | |
| | | ACCCGGGTGCGGTAATTGAAGATCTGTCCGGTCTGCTGGATATG | |
| | | TTCATGCTGTGATAA | |
| 446-6 | E41D, F55Y, S59T, | ATGCACCACCATCATCATCACATCAAGTATAAAGCCGTGTTTTTC | 89 |
| | S65A, R71A, Y89F, | GACTTCGACTATACGTTGGCCGATAGCAGCAAAGCTGTTATTGA | |
| | K101Q, D124H, | GTGCATCAATTATGCGCTGCAGAAAATGGGTTACCCGGAGAGCA | |
| | E137D, A140T, | GCCCGGACTCTATTTGTCGTACCATTGGCCTGACGTTGGCAGAG | |
| | H142P, V134I | GCATACAAAATCCTGACCGGCGATACCAGCGACGCGAACGCAG | |
| | | ATCTGTTTGCGCAATACTTTAAGGAACGTGCGGATCTGGTTATGT | |
| | | GTGACCGCACCGTTATGTTCAGCACCGTTGAGTGCGTCCTGAAG | |
| | | AAACTGAAACAGGCCGATGTTAAGACCGGTATTGTCTCCACGAA | |
| | | ATACCGCTACCGCATCGAAGATATCTTAAAGCGTCACAAGCTGC | |
| | | TCCAGTATTTTGACGTCATCATCGGTGGCGACGATGTGACCGCG | |
| | | CCGAAGCCAGACCCGGAAGGCCTGCTGAAGGCAATTAGCATGG | |
| | | TCGGTTGCCAAAAGGAAGAGGTTCTGTTCGTTGGTGACAGCACT | |
| | | GTGGACGCGCGTACGGCGAAAAACGCTGGCGTGGACTTTGTGGC | |
| | | AGTCTTGACCGGCACCACCGGTGCGAATGAATTCTCGGAGTACA | |
| | | ACCCGGGTGCGGTAATTGAAGATCTGTCCGGTCTGCTGGATATG | |
| | | TTCATGCTGTGATAA | |
| 446-7 | E41D, F55Y, S59T, | ATGCACCACCATCATCATCACATCAAGTATAAAGCCGTGTTTTTC | 90 |
| | S65A, R71A, Y89F, | GACTTCGACTATACGTTGGCCGATAGCAGCAAAGCTGTTATTGA | |
| | K101Q, D124H, | GTGCATCAATTATGCGCTGCAGAAAATGGGTTACCCGGAGAGCA | |
| | E137D, A140T, | GCCCGGACTCTATTTGTCGTACCATTGGCCTGACGTTGGCAGAG | |
| | H142P, D119A, | GCATACAAAATCCTGACCGGCGATACCAGCGACGCGAACGCAG | |
| | V134I | ATCTGTTTGCGCAATACTTTAAGGAACGTGCGGATCTGGTTATGT | |
| | | GTGACCGCACCGTTATGTTCAGCACCGTTGAGTGCGTCCTGAAG | |
| | | AAACTGAAACAGGCCGATGTTAAGACCGGTATTGTCTCCACGAA | |
| | | ATACCGCTACCGCATCGAAGCGATCTTAAAGCGTCACAAGCTGC | |
| | | TCCAGTATTTTGACGTCATCATCGGTGGCGACGATGTGACCGCG | |
| | | CCGAAGCCAGACCCGGAAGGCCTGCTGAAGGCAATTAGCATGG | |
| | | TCGGTTGCCAAAAGGAAGAGGTTCTGTTCGTTGGTGACAGCACT | |
| | | GTGGACGCGCGTACGGCGAAAAACGCTGGCGTGGACTTTGTGGC | |
| | | AGTCTTGACCGGCACCACCGGTGCGAATGAATTCTCGGAGTACA | |
| | | ACCCGGGTGCGGTAATTGAAGATCTGTCCGGTCTGCTGGATATG | |
| | | TTCATGCTGTGATAA | |

### Example 2. Increased Specificity and Thermostability of Selected A6PP Proteins

Experiments were performed to determine the substrate specificity and thermostability of selected A6PP proteins—wild-type Clostridium thermocellum (Cthe); V382 (A6PP comprising S38C, E41D, S59T, R71A, Y89F, D124H, A140T, H142P, and E206P substitutions, relative to SEQ ID NO: 1); V375 (A6PP comprising S38C, E41D, T50S, S59T, D119A, D124H, A140T, H142P, and E206P substitutions, relative to SEQ ID NO: 1); V377 (A6PP comprising S38C, E41D, D119A, D124H, A140T, H142P, L150N, S197A, and E206P substitutions, relative to SEQ ID NO: 1); V413 (A6PP comprising E41D, F55Y, S59T, R71A, Y89F, D124H, A140T, and H142P substitutions, relative to SEQ ID NO: 1) and V446 (A6PP comprising E41D, F55Y, S59T, S65A, R71A, Y89F, K101Q, D124H, E137D, A140T, and H142P substitutions, relative to SEQ ID NO: 1). All A6PP proteins were expressed using standard in vitro protein production methods. All enzymatic reactions described below were performed using a buffer comprising 2 mM MgCl₂, 2 mM MnCl₂, 0.5 mM CoCl₂, 40 mM NaCl, 5% high DE maltodextrin, and 50 mM MES at pH 6.5.

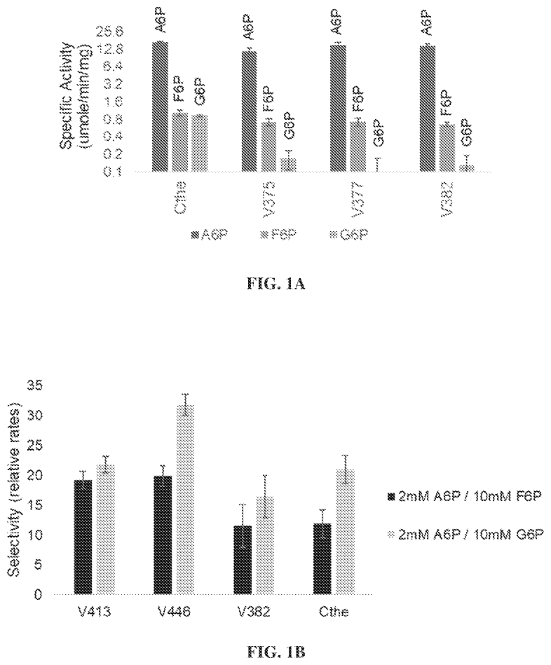

The selected A6PP proteins were individually tested for their abilities to act enzymatically upon different substrates (allulose-6-phosphate (A6P), fructose-6-phosphate (F6P), glucose-6-phosphate (G6P), and glucose-1-phosphate (GIP)). Enzymatic reactions to test for specific activity against A6P were performed by incubating an A6PP with 2 mM A6P at 60° C. for 15 minutes. Enzymatic reactions to test for specific activity against F6P, G6P, and GIP were performed by incubating an A6PP with 10 mM of substrate (F6P, G6P, or GIP) at 60° C. for 60 minutes. The rate of each reaction was determined by measuring the turnover of the substrate (release of phosphate); and specific activity (μmole/min/mg) was determined by dividing substrate turnover by the amount of A6PP estimated by PAGE densitometry. Selectivity of individual A6PPs for A6P substrate relative to F6P and G6P was subsequently determined by dividing the specific activity of an A6PP for A6P by the specific activity of the A6PP for F6P or G6P. As shown in FIGS. 1A-1B, V375 (A6PP comprising S38C, E41D, T50S, S59T, D119A, D124H, A140T, H142P, and E206P substitutions, relative to SEQ ID NO: 1), V377 (A6PP comprising S38C, E41D, D119A, D124H, A140T, H142P, L150N, S197A, and E206P substitutions, relative to SEQ ID NO: 1), V382 (A6PP comprising S38C, E41D, S59T, R71A, Y89F, D124H, A140T, H142P, and E206P substitutions, relative to SEQ ID NO: 1) and V446 (A6PP comprising E41D, F55Y, S59T, S65A, R71A, Y89F, K101Q, D124H, E137D, A140T, and H142P substitutions, relative to SEQ ID NO: 1) were more selective for A6P relative to G6P than wild-type Cthe enzyme (amino acid sequence of SEQ ID NO: 1). V413 (A6PP comprising E41D, F55Y, S59T, R71A, Y89F, D124H, A140T, and H142P substitutions, relative to SEQ ID NO: 1) was more selective for A6P relative to F6P than wild-type Cthe enzyme (amino acid sequence of SEQ ID NO: 1). None of the tested mutant A6PP proteins had any activity against G1P.

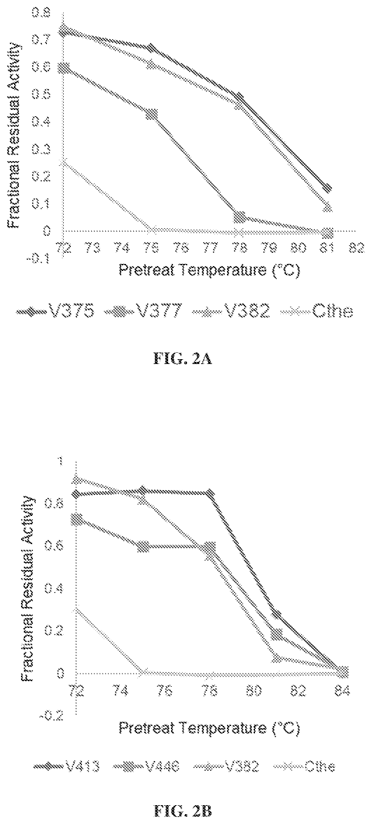

The selected A6PP proteins were individually tested for their thermostability. The A6PP proteins were pre-treated (incubated) at various temperatures from 72-84° C. for 60 minutes. Following this heating step, A6PP proteins were incubated with 2 mM A6P at 60° C. for 15 minutes. Specific activity of each A6PP to utilize A6P as a substrate was determined. Control experiments were performed using the same enzymatic reaction conditions with A6PP proteins that had not been subjected to the heating step. The fractional residual activity of each protein (a measure of thermostability) was determined by comparing the specific activity of an A6PP after heating at various temperatures relative to the specific activity of the control A6PP (i.e., not subject to the heating step). A fractional residual activity of 1.0 (100% residual activity) would indicate that a protein did not lose any enzymatic function as a result of the heating step. Conversely, a residual activity of 0.0 (0% residual activity) would indicate that a protein lost all enzymatic function as a result of the heating step. As shown in FIGS. 2A-2B, each of V375 (A6PP comprising S38C, E41D, T50S, S59T, D119A, D124H, A140T, H142P, and E206P substitutions, relative to SEQ ID NO: 1), V377 (A6PP comprising S38C, E41D, D119A, D124H, A140T, H142P, L150N, S197A, and E206P substitutions, relative to SEQ ID NO: 1), V382 (A6PP comprising S38C, E41D, S59T, R71A, Y89F, D124H, A140T, H142P, and E206P substitutions, relative to SEQ ID NO: 1), V446 (A6PP comprising E41D, F55Y, S59T, S65A, R71A, Y89F, K101Q, D124H, E137D, A140T, and H142P substitutions, relative to SEQ ID NO: 1), and V413 (A6PP comprising E41D, F55Y, S59T, R71A, Y89F, D124H, A140T, and H142P substitutions, relative to SEQ ID NO: 1) maintained residual activity following pre-treatment (heating) at various temperatures than was higher than the wild-type Cthe enzyme (amino acid sequence of SEQ ID NO: 1). For example, V382 maintained about 70% residual activity after heating at 72° C. and about 10% residual activity after heating at 81° C.; V466 maintained about 70% residual activity after heating at 72° C. and about 10% residual activity after heating at 81° C.; and V413 maintained about 85% residual activity after heating at 72° C. and about 30% residual activity after heating at 81° C. Wild-type Cthe enzyme only maintained about 30% residual activity after heating at 72° C. and lost all residual activity after heating at any temperature higher than 75° C.

Additional mutant A6PP proteins (V415, V422, and V429) were expressed and tested as described above. Each of V415, V422, and V429 comprised E41D, S59T, R71A, Y89F, D124H, A140T, H142P, and E206P substitutions, relative to SEQ ID NO: 1. V415 further comprised F55Y and K101Q substitutions, relative to SEQ ID NO: 1. V422 further comprised S38C, F55Y and K101Q substitutions, relative to SEQ ID NO: 1. V429 further comprised F55Y, V134I, and E137D substitutions, relative to SEQ ID NO: 1.

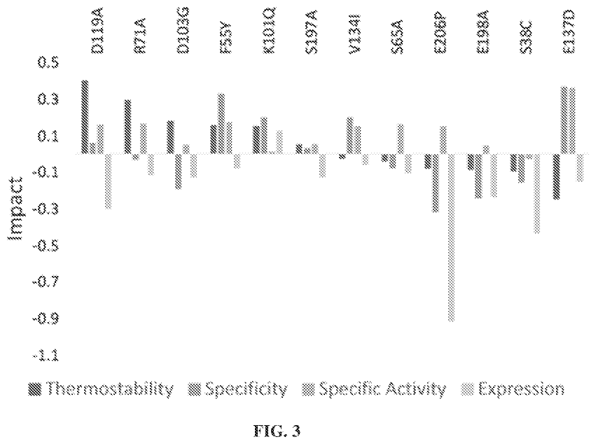

Data for all proteins was modeled to provide a graphical representation of the impact of specific A6PP substitutions on thermostability, specificity, specific activity, and protein expression (FIG. 3). The impact of specific A6PP substitutions were determined using several of the analytical techniques as described in Liao, J. et al., BMC Biotechnology 2007, 7:16 doi:10.1186/1472-6750-7-16.

The D119A, R71A, F55Y, K101Q, and E137D substitutions were found to have the highest aggregate impacts on thermostability, specificity, and specific activity.

These data demonstrate that the mutant A6PP proteins of the disclosure are more selective for allulose-6-phosphate and more thermostable than wild-type A6PP (having an amino acid sequence of SEQ ID NO: 1).

### EQUIVALENTS AND SCOPE

In the claims or description, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should also be understood that, unless clearly indicated to the contrary, in any methods claimed herein that include more than one step or act, the order of the steps or acts of the method is not necessarily limited to the order in which the steps or acts of the method are recited. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A mutant allulose 6-phosphate phosphatase (A6PP) comprising an amino acid sequence at least 85% identical to SEQ ID NO: 1 and further comprising one or more amino acid mutations at positions selected from the group consisting of: amino acid positions 41, 59, 89, 124, 140, and 142 of SEQ ID NO: 1.

2. The mutant A6PP of claim 1, wherein the one or more amino acid mutations are selected from the group consisting of:
(i) an aspartic acid (D) substitution at amino acid position 41 of SEQ ID NO: 1;
(ii) a threonine (T) substitution at amino acid position 59 of SEQ ID NO: 1;
(iii) a phenylalanine (F) substitution at amino acid position 89 of SEQ ID NO: 1;
(iv) a histidine (H) substitution at amino acid position 124 of SEQ ID NO: 1;
(v) a threonine (T) substitution at amino acid position 140 of SEQ ID NO: 1; and
(vi) a proline (P) substitution at amino acid position 142 of SEQ ID NO: 1.

3. The mutant A6PP of claim 2, wherein the one or more amino acid mutations comprise each of the following:
(i) an aspartic acid (D) substitution at amino acid position 41 of SEQ ID NO: 1;
(ii) a threonine (T) substitution at amino acid position 59 of SEQ ID NO: 1;
(iii) a phenylalanine (F) substitution at amino acid position 89 of SEQ ID NO: 1;
(iv) a histidine (H) substitution at amino acid position 124 of SEQ ID NO: 1;
(v) a threonine (T) substitution at amino acid position 140 of SEQ ID NO: 1; and
(vi) a proline (P) substitution at amino acid position 142 of SEQ ID NO: 1.

4. The mutant A6PP of claim 1 or 2, wherein the one or more amino acid mutations are selected from the group consisting of: E41D, S59T, Y89F, D124H, A140T, and H142P.

5. The mutant A6PP of any one of claims 1-4, wherein the one or more amino acid mutations comprise each of the following: E41D, S59T, Y89F, D124H, A140T, and H142P.

6. The mutant A6PP of any one of claims 1-5, wherein the mutant A6PP comprises an amino acid sequence at least 90% identical to SEQ ID NO: 1.

7. The mutant A6PP of any one of claims 1-6, wherein the mutant A6PP comprises an amino acid sequence at least 95% identical to SEQ ID NO: 1.

8. The mutant A6PP of claim 1, wherein the mutant A6PP is at least 85% identical to SEQ ID NO: 2.

9. A mutant allulose 6-phosphate phosphatase (A6PP) comprising the amino acid sequence of SEQ ID NO: 2.

10. The mutant A6PP of any one of claims 1-9 further comprising one or more additional amino acid mutations.

11. The mutant A6PP of claim 10, wherein the one or more additional amino acid mutations are at positions selected from the group consisting of: amino acid positions 38, 55, 65, 71, 101, 103, 119, 134, 137, 197, 198, and 206 of SEQ ID NO: 1.

12. The mutant A6PP of claim 10 or 11, wherein the one or more additional amino acid mutations are selected from the group consisting of:
(a) a cysteine (C) substitution at amino acid position 38 of SEQ ID NO: 1;
(b) a tyrosine (Y) substitution at amino acid position 55 of SEQ ID NO: 1;
(c) an alanine (A) substitution at amino acid position 65 of SEQ ID NO: 1;
(d) an alanine (A) substitution at amino acid position 71 of SEQ ID NO: 1;
(e) a glutamine (Q) substitution at amino acid position 101 of SEQ ID NO: 1;
(f) a glycine (G) substitution at amino acid position 103 of SEQ ID NO: 1;
(g) an alanine (A) substitution at amino acid position 119 of SEQ ID NO: 1;
(h) an isoleucine (I) substitution at amino acid position 134 of SEQ ID NO: 1;
(i) an aspartic acid (D) substitution at amino acid position 137 of SEQ ID NO: 1;
(j) an alanine (A) substitution at amino acid position 197 of SEQ ID NO: 1;
(k) an alanine (A) substitution at amino acid position 198 of SEQ ID NO: 1; and
(l) a proline (P) substitution at amino acid position 206 of SEQ ID NO: 1.

13. The mutant A6PP of any one of claims 10-12, wherein the one or more additional amino acid mutations are selected from the group consisting of S38C, F55Y, S65A, R71A, K101Q, D103G, D119A, V134I, E137D, S197A, E198A, and E206P.

14. The mutant A6PP of claim 13, wherein the one or more additional amino acid mutations comprise two additional amino acid mutations selected from the group consisting of F55Y, R71A, K101Q, D119A, and E137D.

15. The mutant A6PP of claim 14, wherein the one or more additional amino acid mutations comprise three additional amino acid mutations selected from the group consisting of F55Y, R71A, K101Q, D119A, and E137D.

16. The mutant A6PP of claim 15, wherein the one or more additional amino acid mutations comprise four additional amino acid mutations selected from the group consisting of F55Y, R71A, K101Q, D119A, and E137D.

17. The mutant A6PP of claim 16, wherein the one or more additional amino acid mutations comprise each of the following: F55Y, R71A, K101Q, D119A, and E137D.

18. The mutant A6PP of any one of claims 1-17, wherein the mutant A6PP comprises the amino acid sequence of any one of SEQ ID NOs: 2-24.

19. A mutant allulose 6-phosphate phosphatase (A6PP) comprising an amino acid sequence at least 85% identical to SEQ ID NO: 1 and further comprising one or more amino acid mutations at positions selected from the group consisting of: amino acid positions 38, 41, 124, 140, 142 and 206 of SEQ ID NO: 1.

20. The mutant A6PP of claim 19, wherein the one or more amino acid mutations are selected from the group consisting of:
(i) a cysteine (C) substitution at amino acid position 38 of SEQ ID NO: 1;
(ii) an aspartic acid (D) substitution at amino acid position 41 of SEQ ID NO: 1;
(iii) a histidine (H) substitution at amino acid position 124 of SEQ ID NO: 1;
(iv) a threonine (T) substitution at amino acid position 140 of SEQ ID NO: 1;
(v) a proline (P) substitution at amino acid position 142 of SEQ ID NO: 1; and
(vi) a proline (P) substitution at amino acid position 206 of SEQ ID NO: 1.

21. The mutant A6PP of claim 20, wherein the one or more amino acid mutations comprise each of the following:
(i) a cysteine (C) substitution at amino acid position 38 of SEQ ID NO: 1;
(ii) an aspartic acid (D) substitution at amino acid position 41 of SEQ ID NO: 1;
(iii) a histidine (H) substitution at amino acid position 124 of SEQ ID NO: 1;
(iv) a threonine (T) substitution at amino acid position 140 of SEQ ID NO: 1;
(v) a proline (P) substitution at amino acid position 142 of SEQ ID NO: 1; and
(vi) a proline (P) substitution at amino acid position 206 of SEQ ID NO: 1.

22. The mutant A6PP of any one of claims 19-21, wherein the one or more amino acid mutations are selected from the group consisting of: S38C, E41D, D124H, A140T, H142P, and E206P.

23. The mutant A6PP of any one of claims 19-22, wherein the one or more amino acid mutations comprise each of the following: S38C, E41D, D124H, A140T, H142P, and E206P.

24. The mutant A6PP of any one of claims 19-23, wherein the mutant A6PP comprises an amino acid sequence at least 90% identical to SEQ ID NO: 1.

25. The mutant A6PP of any one of claims 19-24, wherein the mutant A6PP comprises an amino acid sequence at least 95% identical to SEQ ID NO: 1.

26. The mutant A6PP of any one of claims 19-25 further comprising one or more additional amino acid mutations.

27. The mutant A6PP of claim 26, wherein the one or more additional amino acid mutations are at positions selected from the group consisting of: amino acid positions 50, 59, 71, 119, 150, and 197 of SEQ ID NO: 1.

28. The mutant A6PP of claim 26 or 27, wherein the one or more additional amino acid mutations are selected from the group consisting of:
(a) a serine (S) substitution at amino acid position 50 of SEQ ID NO: 1;
(b) a threonine (T) substitution at amino acid position 59 of SEQ ID NO: 1;
(c) an alanine (A) substitution at amino acid position 71 of SEQ ID NO: 1;
(d) an alanine (A) substitution at amino acid position 119 of SEQ ID NO: 1;
(e) an asparagine (N) substitution at amino acid position 150 of SEQ ID NO: 1; and
(f) an alanine (A) substitution at amino acid position 197 of SEQ ID NO: 1.

29. The mutant A6PP of any one of claims 26-28, wherein the one or more additional amino acid mutations are selected from the group consisting of T50S, S59T, R71A, D119A, L150N, and S197A.

30. The mutant A6PP of any one of claims 19-29, wherein the mutant A6PP comprises the amino acid sequence of any one of SEQ ID NOs: 24-26.

31. A mutant allulose 6-phosphate phosphatase (A6PP) comprising an amino acid sequence of any one of SEQ ID NOs: 2-26.

32. The mutant A6PP of any preceding claim, wherein the mutant A6PP has a half-life of at least about two hours at about 60° C.

33. The mutant A6PP of any preceding claim, wherein the mutant A6PP has a longer half-life than an A6PP having the amino acid sequence of SEQ ID NO: 1.

34. The mutant A6PP of any preceding claim, wherein the mutant A6PP is more selective for A6P relative to fructose 6-phosphate and/or glucose 6-phosphate than an A6PP having the amino acid sequence of SEQ ID NO: 1.

35. A nucleic acid encoding the mutant A6PP of any one of claims 1-34.

36. A nucleic acid comprising a nucleotide sequence at least 85% identical to any one of SEQ ID NO: 47-69.

37. A method of producing allulose comprising:
converting allulose 6-phosphate (A6P) to allulose catalyzed by the mutant A6PP of any one of claims 1-34.

38. A method for producing allulose, the method comprising:
converting allulose-6-phosphate (A6P) to allulose catalyzed using a mutant allulose 6-phosphate phosphatase (A6PP), wherein the mutant A6PP is encoded by the nucleic acid of claim 35 or 36.

39. The method of claim 38, wherein the nucleic acid is expressed in a microbial cell

40. The method of any one of claims 37-39 further comprising converting fructose 6-phosphate (F6P) to allulose 6-phosphate (A6P) using an allulose 6-phosphate epimerase (A6PE).

41. The method of claim 40 further comprising converting glucose 6-phosphate (G6P) to fructose 6-phoshpate (F6P) using a phosphoglucoisomerase.

42. The method of claim 41 further comprising converting glucose 1-phosphate (G1P) to produce glucose 6-phosphate (G6P) using a phosphoglucomutase

43. The method of claim 42 further comprising converting a polymeric glucose carbohydrate to glucose 1-phosphate (G1P) using an α-glucan or a cellodextrin phosphorylase.

44. A cell comprising the mutant A6PP of any one of claims 1-34 or the nucleic acid of claim 35 or 36.

45. A cell lysate comprising the mutant A6PP of any one of claims 1-34 or the nucleic acid of claim 35 or 36.

46. A kit comprising:
(i) the mutant A6PP of any one of claims 1-34; and
(ii) a reaction buffer.
